(19) United States
(12) Patent Application Publication
(10) Pub. No.: US 2025/0212684 A1
(43) Pub. Date: Jun. 26, 2025

(54) PHOTOELECTRIC CONVERSION ELEMENT, IMAGING ELEMENT, OPTICAL SENSOR, COMPOUND, AND MANUFACTURING METHOD OF COMPOUND

(71) Applicant: FUJIFILM CORPORATION, Tokyo (JP)

(72) Inventors: Yuko Suzuki, Kanagawa (JP); Yosuke Yamamoto, Kanagawa (JP); Hiroki Sugiura, Kanagawa (JP); Hidenobu Kuniyoshi, Kanagawa (JP)

(21) Appl. No.: 19/080,412

(22) Filed: Mar. 14, 2025

Related U.S. Application Data
(63) Continuation of application No. PCT/JP2023/035018, filed on Sep. 26, 2023.

(30) Foreign Application Priority Data
Sep. 30, 2022 (JP) 2022-157552

(51) Int. Cl. H10K 85/60; C07D 209/86; C07D 403/14; C07D 519/00; H10K 30/30; H10K 30/60; H10K 39/32; H10K 85/40; H10K 101/30

(52) U.S. Cl. CPC H10K 85/6576; C07D 209/86; C07D 403/14; C07D 519/00; H10K 85/40; H10K 85/622; H10K 85/623; H10K 85/624; H10K 85/633; H10K 85/654; H10K 85/655; H10K 85/6572; H10K 85/6574; H10K 30/353; H10K 30/60; H10K 39/32; H10K 2101/30

(57) ABSTRACT

The present invention provides a photoelectric conversion element having excellent quantum efficiency in a case of receiving blue light. In addition, an imaging element, an optical sensor, a compound, and a manufacturing method of a compound, which are related to the photoelectric conversion element, are provided. The photoelectric conversion element according to the present invention is a photoelectric conversion element including a conductive film, a photoelectric conversion film, and a transparent conductive film in this order, in which the photoelectric conversion film contains a compound represented by Formula (1).

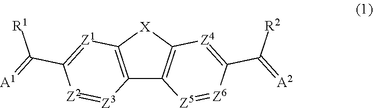

### CROSS-REFERENCE TO RELATED APPLICATIONS

This application is a Continuation of PCT International Application No. PCT/JP2023/035018 filed on Sep. 26, 2023, which claims priority under 35 U.S.C. § 119(a) to Japanese Patent Application No. 2022-157552 filed on Sep. 30, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, a compound, and a manufacturing method of compound.

### 2. Description of the Related Art

In recent years, the development of an element (for example, an imaging element) having a photoelectric conversion film has been progressing.

For example, in Wurthner et al., org. chem. front. 2016, 3, 545-555, an acceptor-donor-acceptor (ADA) type coloring agent that can be applied as a p-type semiconductor or an n-type semiconductor is disclosed.

### SUMMARY OF THE INVENTION

In recent years, along with the demand for improving the performance of imaging elements, optical sensors, and the like, further improvements are required for various characteristics required for photoelectric conversion elements used therein.

For example, a high level of quantum efficiency in a case where the photoelectric conversion element receives blue light (particularly, having a wavelength of 460 nm) is required at a higher level. Here, the blue light refers to light in a wavelength range of 400 to 500 nm.

As a result of studying a photoelectric conversion element containing the ADA-type coloring agent described in Wurthner et al., org. chem. front. 2016, 3, 545-555 as a p-type semiconductor, the present inventors have found that there is room for further improvement in the quantum efficiency in a case of receiving the above-described blue light.

Therefore, an object of the present invention is to provide a photoelectric conversion element having excellent quantum efficiency in a case of receiving blue light.

In addition, another object of the present invention is to provide an imaging element, an optical sensor, a compound, and a manufacturing method of a compound, which are related to the photoelectric conversion element.

The present inventors conducted a thorough investigation to achieve the objects, thereby completing the present invention. That is, the present inventors have found that the objects are achieved by the following configuration.

[1]A photoelectric conversion element including in the following order, a conductive film, a photoelectric conversion film, and a transparent conductive film, in which the photoelectric conversion film contains a compound represented by Formula (1).

[2] The photoelectric conversion element according to [1], in which the substituent selected from the substituent group S represents a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{A}M described later, a group represented by Formula (S-3), or a group represented by Formula (S-4).

[3] The photoelectric conversion element according to [1] or [2], in which the group represented by Formula (A-1) is a group represented by Formula (A-2).

[4] The photoelectric conversion element according to [3], in which the group represented by Formula (A-2) is a group represented by Formula (C-1) or a group represented by Formula (C-2).

[5] The photoelectric conversion element according to any one of [1] to [4], in which X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—, and
R^{N} and R^{C1} to R^{C10} have the same meanings as R^{N} and R^{C1} to R^{C10} in Formula (1).

[6] The photoelectric conversion element according to any one of [1] to [5], in which X represents >NR^{N}, >CR^{C1}R^{C2}, or >C═CR^{C3}R^{C4}, and
R^{N} and R^{C1} to R^{C4} have the same meanings as R^{N} and R^{C1} to R^{C4} in Formula (1).

[7] The photoelectric conversion element according to any one of [1] to [6], in which X represents >NR^{N}, and
R^{N} has the same meaning as R^{N} in Formula (1).

[8] The photoelectric conversion element according to any one of [1] to [7], in which the photoelectric conversion film further contains an n-type organic semiconductor, and the photoelectric conversion film has a bulk hetero structure formed in a state where the compound represented by Formula (1) and the n-type organic semiconductor are mixed.

[9] The photoelectric conversion element according to [8], in which the n-type organic semiconductor includes fullerenes selected from the group consisting of a fullerene and a derivative of the fullerene.

[10] The photoelectric conversion element according to any one of [1] to [9], in which the photoelectric conversion film further contains a coloring agent.

[11] The photoelectric conversion element according to any one of [1] to [10], in which the photoelectric conversion film further contains a p-type organic semiconductor.

[12] The photoelectric conversion element according to any one of [1] to [11], further comprising one or more interlayers between the conductive film and the transparent conductive film, in addition to the photoelectric conversion film.

[13] An imaging element comprising the photoelectric conversion element according to any one of [1] to [12].

[14] An optical sensor comprising the photoelectric conversion element according to any one of [1] to [12].

[15] A compound represented by Formula (1).

[16] The compound according to [15], in which the substituent selected from the substituent group S represents a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{Ar1} described later, a group represented by Formula (S-3), or a group represented by Formula (S-4).

[17] The compound according to [15] or [16], in which the group represented by Formula (A-1) is a group represented by Formula (A-2).

[18] The compound according to [17], in which the group represented by Formula (A-2) is a group represented by Formula (C-1) or a group represented by Formula (C-2).

[19] The compound according to any one of [15] to [18], in which X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—, and
R^{N} and R^{C1} to R^{C10} have the same meanings as R^{N} and R^{C1} to R^{C10} in Formula (1).

[20] The compound according to any one of [15] to [19], in which X represents >NR^{N}>CR^{C1}R^{C2}, or >C═CR^{C3}R^{C4}, and
R^{N} and R^{C1} to R^{C4} have the same meanings as R^{N} and R^{C1} to R^{C4} in Formula (1).

[21] The compound according to any one of [15] to [20], in which X represents >NR^{N} and
R^{N} has the same meaning as R^{N} in Formula (1).

[22] A compound represented by Formula (2).

[23] A manufacturing method of a compound, including a step of reacting a compound represented by Formula (2a) with a compound represented by Formula (X) to manufacture a compound represented by Formula (2b).

[24] A manufacturing method of a compound, including a step of reacting a compound represented by Formula (2a) with a compound represented by Formula (X) to manufacture a compound represented by Formula (2b), and a step of converting a group represented by R^{L4} and a group represented by R^{L5} in the compound represented by Formula (2b) into a formyl group, *—Sn(R^{Sn})₃, *—B(R^{B1})₂, or *—B⁻(R^{B2})₃M⁺,
R^{Sn}, R^{B1}, and R^{B2} each independently represent a substituent, and a plurality of R^{Sn}'s, R^{B1}'s, and R^{B2}'s may be the same as or different from each other, R^{B1}'s and R^{B2}'s may be bonded to each other to form a ring structure, M⁺ represents a monovalent metal cation, and * represents a bonding position.

[25] A compound represented by Formula (3).

[26] A manufacturing method of a compound, including a step 1 of reacting a compound represented by Formula (3a) with a compound represented by Formula (A) to obtain a compound represented by Formula (3b), which has a protective group represented by SiR^{Y1}₃, a step 2 of reacting the compound represented by Formula (3b) with a metalating reagent, reacting the reacted compound with a formylating agent, and further deprotecting the protective group to obtain a compound represented by Formula (3c), and a step 3 of reacting the compound represented by Formula (3c) with a compound represented by Formula (C) to obtain a compound represented by Formula (3).

[27] A compound represented by Formula (3c).

According to the present invention, it is possible to provide a photoelectric conversion element having excellent quantum efficiency in a case of receiving blue light.

In addition, according to the present invention, it is possible to provide an imaging element, an optical sensor, a compound, and a manufacturing method of a compound, which are related to the photoelectric conversion element.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

Hereinafter, the meaning of each description in the present specification will be expressed.

In the present specification, numerical ranges represented by “to” include numerical values before and after “to” as lower limit values and upper limit values.

In the present specification, a hydrogen atom may be a light hydrogen atom (an ordinary hydrogen atom) or a deuterium atom (for example, a double hydrogen atom and the like).

A symbol “*” specified in a chemical formula represents a bonding position unless otherwise specified.

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a “substituent” includes a group exemplified by a substituent W described later, unless otherwise specified.

### (Substituent W)

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (a heterocyclic group), a cyano group, a nitro group, an alkoxy group, an aryloxy group, a silyl group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a carboxy group, a phosphoric acid group, a sulfonic acid group, a hydroxy group, a thiol group, an acylamino group, a carbamoyl group, a ureido group, a boronic acid group, and a primary amino group.

Each of the above-described groups may further have a substituent (for example, one or more groups of each of the above-described groups, and the like), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound described later preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, examples of the halogen atom include a fluorine atom, a chlorine atom, a bromine atom, and an iodine atom.

In the present specification, the aliphatic hydrocarbon group may be linear, branched, or cyclic.

Examples of the above-described aliphatic hydrocarbon group include an alkyl group, an alkenyl group, and an alkynyl group.

In the present specification, unless otherwise specified, the number of carbon atoms of the alkyl group is preferably 1 to 20, more preferably 1 to 10, and still more preferably 1 to 6.

The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be any of a cycloalkyl group, a bicycloalkyl group, or a tricycloalkyl group, and may have a ring structure thereof as a partial structure.

In the alkyl group which may have a substituent, examples of the substituent which may be contained in the alkyl group include the group exemplified by the substituent W. Among these, an aryl group (preferably having 6 to 18 carbon atoms and more preferably having 6 carbon atoms), a heteroaryl group (preferably having 5 to 18 carbon atoms and more preferably having 5 or 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The number of carbon atoms of the alkenyl group is preferably 2 to 20. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The number of carbon atoms of the alkynyl group is preferably 2 to 20. In the alkynyl group which may have a substituent, the substituent which may be contained in the alkynyl group includes the same examples as the substituent in the alkyl group which may have a substituent.

In the present specification, an aromatic ring constituting the aromatic ring structure or the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the above-described aromatic ring is preferably 4 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, benzo[1,2-b:4,5-b′]dithiophene ring and the like), a thienothiophene ring (for example, thieno[3,2-b]thiophene ring and the like), a thiazolothiazole ring (for example, thiazolo[5,4-d]thiazole ring and the like), a naphthodithiophene ring (for example, a naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, and the like), a benzothienobenzothiophene ring, a dithieno[3,2-b:2′,3′-d]thiophene ring, and a 3,4,7,8-tetrathiadicyclopenta[a,e]pentalene ring.

In the aromatic ring which may have a substituent, examples of the type of the substituent which may be contained in the aromatic ring include a group exemplified by the substituent W. In a case where the aromatic ring has substituents, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, examples of a type of the substituents that these groups may have include a group exemplified by the substituent W. In a case where these groups each of which may have a substituent have substituents, the number of substituents may be 1 or more (for example, 1 to 4 or the like).

In the present specification, the number of ring members in the aliphatic heterocyclic group is preferably 5 to 20, more preferably 5 to 12, and still more preferably 6 to 8.

Examples of the heteroatom which is contained in the aliphatic heterocyclic group include a sulfur atom, an oxygen atom, a nitrogen atom, a selenium atom, a tellurium atom, a phosphorus atom, a silicon atom, and a boron atom, and a sulfur atom, an oxygen atom, or a nitrogen atom is preferable.

Examples of the aliphatic heterocyclic ring constituting of the above-described aliphatic heterocyclic group include a pyrrolidine ring, an oxolane ring, a thiolane ring, a piperidine ring, a tetrahydrofuran ring, a tetrahydropyran ring, a thiane ring, a piperazine ring, a morpholine ring, a quinuclidine ring, a pyrrolidine ring, an azetidine ring, an oxetane ring, an aziridine ring, a dioxane ring, a pentamethylene sulfide ring, and γ-butyrolactone ring.

The bonding direction of the divalent group (for example, —CO—O— and the like) denoted in the present specification, is not limited unless otherwise specified. For example, in a case where Y in a compound represented by a formula “X—Y—Z” is —CO—O—, the compound may be any of “X—O—CO—Z” or “X—CO—O—Z”.

In the present specification, the expression “may have an ethereal oxygen atom” means that an aliphatic hydrocarbon group may have a divalent linking group represented by —O— at an (between carbon atom-carbon atom) or a terminal.

In the present specification, regarding a compound that may have a geometric isomer (cis-trans isomer), a general formula or a structural formula representing the above compound may be described only in the form of either a cis isomer or a trans isomer for convenience. Even in such a case, unless otherwise specified, the form of the compound is not limited to either the cis isomer or the trans isomer, and the compound may be either the cis isomer or the trans isomer.

### [Photoelectric Conversion Element]

The photoelectric conversion element according to an embodiment of the present invention includes a conductive film, a photoelectric conversion film, and a transparent conductive film in this order, in which the photoelectric conversion film contains a compound represented by Formula (1) described later (hereinafter, referred to as a “specific compound”).

The mechanism by which the photoelectric conversion element according to the embodiment of the present invention can solve the above-described problems is not necessarily clear, but the inventors of the present invention assume as follows.

The mechanism by which the effect is obtained is not limited by the following supposition. That is, even in a case where the effect is obtained by a mechanism other than the following, it is included in the scope of the present invention.

The compound disclosed in Wurthner et al., org. chem. front. 2016, 3, 545-555 is an ADA-type coloring agent having a structure in which a branched alkyl group is substituted in a fused-ring structure such as fluorene as a donor site. In a case where the compound has a structure in which aromatic rings are fused, aggregation between coloring agents is likely to occur, which leads to deterioration of the quantum efficiency of the photoelectric conversion element. Therefore, in Wurthner et al., org. chem. front. 2016, 3, 545-555, the aggregation is suppressed by introducing a substituent such as an alkyl group.

However, in the substituent in Wurthner et al., org. chem. front. 2016, 3, 545-555, the substituent is too large. Therefore, the transfer of electrons and holes is not efficiently performed, and the quantum efficiency is still insufficient.

On the other hand, in the case of the specific compound in the present invention, since the size of the substituent introduced in the fused-ring structure such as carbazole and fluorene is optimized, the transfer of electrons or holes can be efficiently performed without the aggregation of the coloring agents as described above. As a result, it is presumed that the quantum efficiency of the photoelectric conversion element is improved as compared with that in Wurthner et al., org. chem. front. 2016, 3, 545-555.

Hereinafter, the fact that the quantum efficiency in a case where the photoelectric conversion element receives blue light (light in a wavelength range of 400 to 500 nm) is more excellent is also referred to as the fact that the effect of the present invention is more excellent.

The configuration of the photoelectric conversion element according to the embodiment of the present invention will be described in detail below.

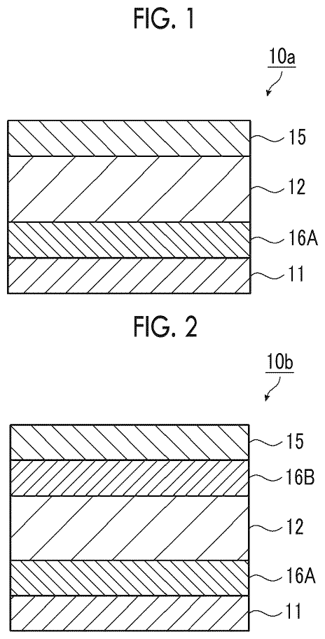

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1×10⁷ V/cm, and still more preferably 1×10⁻³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### [Photoelectric Conversion Film]

The photoelectric conversion element according to the embodiment of the present invention includes a photoelectric conversion film.

### <Specific Compound>

The photoelectric conversion film contains a compound (specific Compound) represented by Formula (1).

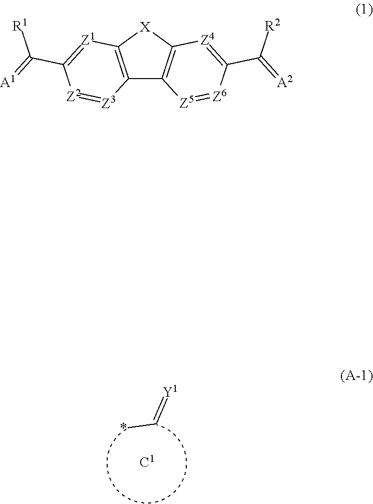

In Formula (1), X represents >NR^{N}, >CR^{C1}R^{C2}, >C═CR^{C3}R^{C4}, >SiR^{C5}R^{C6}, >GeR^{C7}R^{C8}, —OC(R^{C9})(R^{C10})—, a sulfur atom, an oxygen atom, or a selenium atom.

R^{N} represents a substituent selected from a substituent group S described later.

R^{C1} to R^{C10} each independently represent a hydrogen atom or a substituent selected from the substituent group S described later. Provided that at least one of R^{C1} or R^{C2} represents the substituent selected from the substituent group S, at least one of R^{C3} or R^{C4} represents the substituent selected from the substituent group S, at least one of R^{C5} or R^{C6} represents the substituent selected from the substituent group S, at least one of R^{C7} or R^{C8} represents the substituent selected from the substituent group S, and at least one of R^{C9} or R^{C10} represents the substituent selected from the substituent group S.

R^{C1} and R^{C2}, R^{C3} and R^{C4}, Res and R^{C6}, R^{C7} and R^{C8}, and R^{C9} and R^{C10} may each independently be bonded directly or through a linking group to form a ring. For example, both R^{C1} and R^{C2} may be a benzene ring group, and both of them may be directly bonded (bonded through a single bond) to each other to form a fluorene ring.

X preferably represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—, more preferably represents >NR^{N}, >CR^{C1}R^{C2}, or >C═CR^{C3}R^{C4}, still more preferably represents >NR^{N} or >CR^{C1}R^{C2}, and most preferably represents >NR^{N}.

In addition, it is preferable that both R^{C1} and R^{C2} represent a substituent selected from the substituent group S.

Moreover, it is preferable that both R^{C3} and R^{C4} represent a substituent selected from the substituent group S.

Moreover, it is preferable that both R^{C5} and R^{C6} represent a substituent selected from the substituent group S.

Moreover, it is preferable that both R^{C7} and R^{C8} represent a substituent selected from the substituent group S.

Moreover, it is preferable that both R^{C9} and R^{C10} represent a substituent selected from the substituent group S.

Substituent group S

The substituent group S is a group consisting of the following substituents.

The substituent group S: a linear aliphatic hydrocarbon group having 1 to 3 carbon atoms (hereinafter, also referred to as a “substituent S^{A}”), a cyclic aliphatic hydrocarbon group having 3 to 8 carbon atoms, which may have a substituent (hereinafter, also referred to as a “substituent S^{B}”), a linear aliphatic hydrocarbon group having 1 to 3 carbon atoms, which has a cyclic aliphatic hydrocarbon group having 3 to 8 carbon atoms (hereinafter, also referred to as a “substituent S^{AB}”), a linear aliphatic hydrocarbon group having 1 to 3 carbon atoms, which has an aromatic ring group which may have a substituent (hereinafter, also referred to as a “substituent S^{AAr}”), a branched aliphatic hydrocarbon group having 3 carbon atoms, which has a cyclic aliphatic hydrocarbon group having 3 to 8 carbon atoms (hereinafter, also referred to as a “substituent S^{DB}”), a branched aliphatic hydrocarbon group having 3 carbon atoms, which has an aromatic ring group which may have a substituent (hereinafter, also referred to as a “substituent S^{DAr}”), an aromatic ring group which may have a substituent (hereinafter, also referred to as a “substituent S^{Ar}”), a group represented by Formula (S-1), and a group represented by Formula (S-2).

In the substituent group S, the linear aliphatic hydrocarbon group having 1 to 3 carbon atoms, the cyclic aliphatic hydrocarbon group having 3 to 8 carbon atoms, which may have a substituent, the linear aliphatic hydrocarbon group having 1 to 3 carbon atoms, which has a cyclic aliphatic hydrocarbon group having 3 to 8 carbon atoms, the linear aliphatic hydrocarbon group having 1 to 3 carbon atoms, which has an aromatic ring group which may have a substituent, the branched aliphatic hydrocarbon group having 3 carbon atoms, which has a cyclic aliphatic hydrocarbon group having 3 to 8 carbon atoms, and the branched aliphatic hydrocarbon group having 3 carbon atoms, which has an aromatic ring group which may have a substituent, may have an ethereal oxygen atom and may be substituted with a halogen atom.

The number of carbon atoms in the substituent S^{A} is not particularly limited as long as it is 1 to 3, but is preferably 1 or 2.

Examples of the substituent S^{A} include a linear alkyl group having 1 to 3 carbon atoms, a linear alkenyl group having 2 or 3 carbon atoms, and a linear alkynyl group having 2 or 3 carbon atoms.

Examples of the linear alkyl group having 1 to 3 carbon atoms include a methyl group, an ethyl group, and an n-propyl group, and among these, a methyl group or an ethyl group is preferable.

Examples of the linear alkenyl group having 2 or 3 carbon atoms include a vinyl group, an allyl group, and an isoallyl group.

Examples of the linear alkynyl group having 2 or 3 carbon atoms include an ethynyl group, a 1-propynyl group, and a propargyl group.

The cyclic aliphatic hydrocarbon group having 3 to 8 carbon atoms in the substituent S^{B} may have any of monocyclic or polycyclic structures.

The number of carbon atoms in the cyclic aliphatic hydrocarbon group having 3 to 8 carbon atoms in the substituent S^{B} is not particularly limited as long as it is 3 to 8, but is preferably 3 to 6 and more preferably 3.

Examples of the cyclic aliphatic hydrocarbon group having 3 to 8 carbon atoms in the substituent S^{B} include a cyclic alkyl group having 3 to 8 carbon atoms and a cyclic alkenyl group having 3 to 8 carbon atoms.

Examples of the cyclic alkyl group having 3 to 8 carbon atoms include a cyclopropyl group, a cyclobutyl group, a cyclohexyl group, a cycloheptyl group, a 4-tetrahydropyranyl group, and a group obtained by removing one hydrogen atom from bicyclo[1,1,1]pentane.

Examples of the cyclic alkenyl group having 3 to 8 carbon atoms include a group obtained by removing one hydrogen atom from a cycloalkene having 3 to 8 carbon atoms. Examples of the cycloalkene include cyclobutene, cyclopentene, cyclohexene, 1,3-cyclohexadiene, and 1,4-cyclohexadiene.

The number of substituents which may be contained in the cyclic aliphatic hydrocarbon group having 3 to 8 carbon atoms in the substituent S^{B} is not particularly limited, but is preferably 1 to 6, more preferably 1 to 4, and still more preferably 1 or 2.

Examples of the substituent which may be contained in the cyclic aliphatic hydrocarbon group having 3 to 8 carbon atoms in the substituent S^{B} include the groups exemplified as the substituent W.

Among these, as the substituent which may be contained in the cyclic aliphatic hydrocarbon group having 3 to 8 carbon atoms in the substituent S^{B}, a group exemplified by the substituent group R^{Ar1} described later is preferable, and a linear alkyl group having 1 to 3 carbon atoms, a branched alkyl group having 3 to 5 carbon atoms, or a halogen atom is more preferable.

As described above, the substituent S^{AB} is a linear aliphatic hydrocarbon group having 1 to 3 carbon atoms, which has a cyclic aliphatic hydrocarbon group having 3 to 8 carbon atoms, but it corresponds to a group obtained by substituting one or more hydrogen atoms in the substituent S^{A} (linear aliphatic hydrocarbon group having 1 to 3 carbon atoms) with a cyclic aliphatic hydrocarbon group having 3 to 8 carbon atoms.

The number of cyclic aliphatic hydrocarbon groups having 3 to 8 carbon atoms in the substituent S^{AB} is not particularly limited, but is preferably 1 to 3 and more preferably 1 or 2.

Specific aspects and suitable aspects of the linear aliphatic hydrocarbon group having 1 to 3 carbon atoms in the substituent S^{AB} are the same as the specific aspects and suitable aspects of the substituent S^{A} described above.

In addition, specific aspects and suitable aspects of the cyclic aliphatic hydrocarbon group having 3 to 8 carbon atoms in the substituent S^{AB} are the same as the specific aspects and suitable aspects of the cyclic aliphatic hydrocarbon group having 3 to 8 carbon atoms in the substituent S^{B}.

Among these, as the linear aliphatic hydrocarbon group having 1 to 3 carbon atoms in the substituent S^{AB}, a linear alkyl group having 1 to 3 carbon atoms is preferable, and a methyl group or an ethyl group is more preferable. In addition, as the cyclic aliphatic hydrocarbon group having 3 to 8 carbon atoms in the substituent S^{AB}, a cyclic alkyl group having 3 to 8 carbon atoms is preferable, and a cyclic alkyl group having 3 to 6 carbon atoms is more preferable.

Examples of the substituent S^{AB} include a methyl group having a cyclic alkyl group having 3 to 8 carbon atoms (hereinafter, also referred to as a “substituent S^{AB1}” in other words, a group obtained by substituting at least one hydrogen atom of a methyl group with a cyclic alkyl group having 3 to 8 carbon atoms), an ethyl group having a cyclic alkyl group having 3 to 8 carbon atoms (hereinafter, also referred to as a “substituent S^{AB2}”, in other words, a group obtained by substituting a hydrogen atom of an ethyl group with a cyclic alkyl group having 3 to 8 carbon atoms), and an n-propyl group having a cyclic alkyl group having 3 to 8 carbon atoms (hereinafter, also referred to as a “substituent S^{AB3}”, in other words, a group obtained by substituting a hydrogen atom of an n-propyl group with a cyclic alkyl group having 3 to 8 carbon atoms).

Examples of the cyclic alkyl group having 3 to 8 carbon atoms include a cyclopropyl group, a cyclobutyl group, a cyclohexyl group, a cycloheptyl group, a 4-tetrahydropyranyl group, and a group obtained by removing one hydrogen atom from bicyclo[1,1,1]pentane. Among these, a cyclopropyl group is preferable.

The number of cyclic alkyl groups having 3 to 8 carbon atoms in the substituent S^{AB1} is not particularly limited, but is preferably 1 or 2.

Among these, the substituent S^{AB1} is preferably a group obtained by substituting one or two hydrogen atoms of a methyl group with a cyclic alkyl group having 3 to 6 carbon atoms, and more preferably a group obtained by substituting one or two hydrogen atoms of a methyl group with a cyclic alkyl group having 3 carbon atoms (cyclopropyl group).

The number of cyclic alkyl groups having 3 to 8 carbon atoms in the substituent S^{AB2} is not particularly limited, but is preferably 1 or 2.

The number of cyclic alkyl groups having 3 to 8 carbon atoms in the substituent S^{AB3} is not particularly limited, but is preferably 1 or 2.

As described above, the substituent S^{AAr} is a linear aliphatic hydrocarbon group having 1 to 3 carbon atoms, which has an aromatic ring group which may have a substituent, but corresponds to a group obtained by substituting one or more hydrogen atoms in the above-described substituent S^{A} (linear aliphatic hydrocarbon group having 1 to 3 carbon atoms) with a substituent S^{Ar} (aromatic ring group which may have a substituent) described later.

In the substituent S^{AAr}, the number of aromatic ring groups which may have a substituent is not particularly limited, but is preferably 1 to 3 and more preferably 1 or 2.

Specific aspects and suitable aspects of the linear aliphatic hydrocarbon group having 1 to 3 carbon atoms and the aromatic ring group which may have a substituent in the substituent S^{AAr} are the same as the specific aspects and suitable aspects of the substituent S^{A} described above and the substituent S^{Ar} described later. Among these, as the linear aliphatic hydrocarbon group having 1 to 3 carbon atoms in the substituent S^{AAr}, a linear alkyl group having 1 to 3 carbon atoms is preferable, and a methyl group or an ethyl group is more preferable. In addition, the aromatic ring group which may have a substituent in the substituent S^{AAr} is preferably an aryl group and more preferably a phenyl group.

As described above, the substituent S^{DB} is a branched aliphatic hydrocarbon group having 3 carbon atoms, which has a cyclic aliphatic hydrocarbon group having 3 to 8 carbon atoms, but corresponds to a group obtained by substituting one or more hydrogen atoms in the branched aliphatic hydrocarbon group having 3 carbon atoms (hereinafter, also referred to as a “substituent S^{D}”) with a cyclic aliphatic hydrocarbon group having 3 to 8 carbon atoms.

The number of cyclic aliphatic hydrocarbon groups having 3 to 8 carbon atoms in the substituent S^{AD} is not particularly limited, but is preferably 1 to 3 and more preferably 1 or 2.

Examples of the branched aliphatic hydrocarbon group having 3 carbon atoms in the substituent S^{DB} include an isopropyl group and an isopropenyl group. Among these, an isopropyl group is preferable.

In addition, specific aspects and suitable aspects of the cyclic aliphatic hydrocarbon group having 3 to 8 carbon atoms in the substituent S^{DB} are the same as specific aspects and suitable aspects of the cyclic aliphatic hydrocarbon group having 3 to 8 carbon atoms in the substituent S^{B}.

As described above, the substituent S^{DAr} is a branched aliphatic hydrocarbon group having 3 carbon atoms, which has an aromatic ring group which may have a substituent, but corresponds to a group obtained by substituting one or more hydrogen atoms in the above-described substituent S^{D} (branched aliphatic hydrocarbon group having 3 carbon atoms) with a substituent S^{Ar} (aromatic ring group which may have a substituent) described later.

In the substituent S^{DAr}, the number of aromatic ring groups which may have a substituent is not particularly limited, but is preferably 1 to 3 and more preferably 1 or 2.

Specific aspects and suitable aspects of the substituent S^{D} in the substituent S^{DAr} are as described above.

Specific aspects and suitable aspects of the substituent S^{Ar} in the substituent S^{DAr} are as described later.

The aromatic ring constituting the aromatic ring group in the substituent S^{Ar} may be any of a monocyclic ring or a polycyclic ring, and may be any of an aromatic hydrocarbon ring or an aromatic heterocyclic ring. Specific aspects of the monocyclic aromatic ring, the polycyclic aromatic ring, the aromatic hydrocarbon ring, and the aromatic heterocyclic ring are as described above.

The number of ring member atoms in the aromatic ring constituting the aromatic ring group in the substituent S^{Ar} is preferably 4 to 15, more preferably 4 to 10, and still more preferably 4 to 6.

Among these, as the aromatic hydrocarbon ring constituting the aromatic ring group in the substituent S^{Ar}, a benzene ring, a naphthalene ring, or an anthracene ring is preferable.

In addition, as the aromatic heterocyclic ring constituting the aromatic ring group in the substituent S^{Ar}, a pyridine ring, a thiophene ring, a benzofuran ring (for example, a 2,3-benzofuran ring or the like), or a benzothiophene ring (for example, a benzo[b]thiophene ring or the like) is preferable.

The number of substituents which may be contained in the aromatic ring group in the substituent S^{Ar} is not particularly limited, but is preferably 1 to 6, more preferably 1 to 4, and still more preferably 1 or 2.

Examples of the substituent which may be contained in the aromatic ring group in the substituent S^{Ar} include the groups exemplified as the substituent W.

In a case where the aromatic ring group in the substituent S^{Ar} may have a plurality of substituents, the substituents may be bonded to each other to form a non-aromatic ring.

Among these, as the substituent which may be contained in the aromatic ring group in the substituent S^{Ar}, a group exemplified by the substituent group R^{Ar1} described later is preferable, and a linear alkyl group having 1 to 3 carbon atoms, a branched alkyl group having 3 to 5 carbon atoms, or a halogen atom is more preferable.

### Substituent Group RAM

The substituent selected from the substituent group R^{Ar1} is as follows.

The substituent group R^{Ar1}: a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, an aromatic ring group, a halogen atom, and *—Si(R^{Si})₃.

R^{Si} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, or an aromatic ring group.

In the substituent group R^{Ar1}, the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have an ethereal oxygen atom and may be substituted with a halogen atom.

Specific aspects and suitable aspects of the linear aliphatic hydrocarbon group having 1 to 3 carbon atoms and the cyclic aliphatic hydrocarbon group having 3 to 8 carbon atoms in the substituent group R^{Ar1} are the same as the specific aspects and suitable aspects of the substituent S^{A} and the substituent S^{B}.

Examples of the branched aliphatic hydrocarbon group having 3 to 5 carbon atoms in the substituent group R^{Ar1} include a branched alkyl group having 3 to 5 carbon atoms (an isopropyl group and the like), a branched alkenyl group having 3 to 5 carbon atoms, and a branched alkynyl group having 3 to 5 carbon atoms. In addition, the number of carbon atoms in the branched aliphatic hydrocarbon group having 3 to 5 carbon atoms is not particularly limited as long as it is 3 to 5, but is preferably 3 or 4.

The specific aspect and the suitable aspect of the aromatic ring group in the substituent group R^{Ar1} are the same as the specific aspect and the suitable aspect of the aromatic ring group in the substituent S^{Ar}, and among these, an aryl group is preferable and a phenyl group is more preferable.

Specific aspects and suitable aspects of the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms, which are represented by R^{Si}, are the same as the specific aspects and suitable aspects of each group described in the substituent group R^{Ar1}.

In addition, the specific aspect and the suitable aspect of the aromatic ring group represented by R^{Si} are as described above, but among them, an aryl group is preferable, and a phenyl group is more preferable.

The group represented by Formula (S-1) is as follows.

*-L^{S1}-C(R^{S1})₃ Formula (S-1)

In Formula (S-1), L^{S1} represents a single bond or a linear alkylene group having 1 to 3 carbon atoms.

R^{S1}'s each independently represent a hydrogen atom, a linear aliphatic hydrocarbon group having 1 to 3 carbon atoms, a branched aliphatic hydrocarbon group having 3 or 4 carbon atoms, or a cyclic alkyl group having 3 carbon atoms.

A plurality of R^{S1}'s may be the same as or different from each other. Provided that two or more of three R^{S1}'s are not hydrogen atoms.

The alkylene group, the linear aliphatic hydrocarbon group having 1 to 3 carbon atoms, the branched aliphatic hydrocarbon group having 3 or 4 carbon atoms, and the cyclic alkyl group having 3 carbon atoms may have an ethereal oxygen atom and may be substituted with a halogen atom.

The number of carbon atoms in the group represented by Formula (S-1) is preferably 3 to 9 and more preferably 3 to 7.

The number of carbon atoms in the group represented by Formula (S-1) means the total number of all carbon atoms included in the group represented by Formula (S-1).

L^{S1} is preferably a single bond or a methylene group, and more preferably a single bond.

The number of R^{S1}'s represented by atoms or groups other than a hydrogen atom is not particularly limited as long as it is 2 or more, but it is preferable that one of R^{S1}'s is a hydrogen atom and the remaining two are atoms or groups other than a hydrogen atom.

Among these, R^{S1} is preferably a methyl group, an isopropyl group, or a t-butyl group, and more preferably a methyl group or an isopropyl group.

The group represented by Formula (S-2) is as follows.

*—C(=Q)R^{Ac1} Formula (S-2)

In Formula (S-2), Q represents an oxygen atom or a sulfur atom.

Q is preferably an oxygen atom.

R^{Ac1} represents an aliphatic hydrocarbon group which may have a substituent, an aromatic ring group which may have a substituent, or an aliphatic heterocyclic group which may have a substituent. The definition of each group represented by R^{Ac1} is as described above. In addition, examples of the substituent which may be contained in each group represented by R^{Ac1} include the group exemplified as the substituent W.

Among the above, as the aliphatic hydrocarbon group represented by R^{Ac1}, which may have a substituent, a linear, branched, or cyclic aliphatic hydrocarbon group which may have a halogen atom is preferable.

Among the above, as the aromatic ring group represented by R^{Ac1}, which may have a substituent, an aromatic ring group which may have a substituent selected from the substituent group R^{Ar1} is preferable.

From the viewpoint that the effect of the present invention is more excellent, the substituent selected from the substituent group S preferably represent a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{Ar1}, a group represented by Formula (S-3), or a group represented by Formula (S-4).

In addition, specific aspects and suitable aspects of the substituent selected from the substituent group RAM are as described above, but the substituent selected from the substituent group R^{Ar1} is preferably a substituent selected from the substituent group R^{Ar2}.

The substituent group R^{Ar2}: a linear aliphatic hydrocarbon group having 1 or 2 carbon atoms, a branched aliphatic hydrocarbon group having 3 to 4 carbon atoms, a cyclic aliphatic hydrocarbon group having 3 to 6 carbon atoms, a halogen atom, and *—Si(R^{Si})₃.

R^{Si} represents a linear aliphatic hydrocarbon group having 1 or 2 carbon atoms, a branched aliphatic hydrocarbon group having 3 to 4 carbon atoms, a cyclic aliphatic hydrocarbon group having 3 to 6 carbon atoms, or an aromatic ring group.

In the substituent group R^{Ar2}, the linear aliphatic hydrocarbon group having 1 or 2 carbon atoms, the branched aliphatic hydrocarbon group having 3 to 4 carbon atoms, and the cyclic aliphatic hydrocarbon group having 3 to 6 carbon atoms may have an ethereal oxygen atom and may be substituted with a halogen atom.

The group represented by Formula (S-3) is as follows.

*—C(R^{S2})₃ Formula (S-3)

In Formula (S-3), R^{S2}'s each independently represent a hydrogen atom, a methyl group, an isopropyl group, or a t-butyl group.

A plurality of R^{S2}'s may be the same as or different from each other. Provided that the number of carbon atoms in the group represented by Formula (S-3) is 3 to 9, and two or more of three R^{S2}'s are other than a hydrogen atom.

The number of carbon atoms in the group represented by Formula (S-3) is preferably 3 to 9 and more preferably 3 to 7.

The number of carbon atoms in the group represented by Formula (S-3) means the total number of all carbon atoms included in the group represented by Formula (S-3).

The number of R^{S2}'s represented by atoms or groups other than a hydrogen atom is not particularly limited as long as it is 2 or more, but it is preferable that one of R^{S2}'s is a hydrogen atom and the remaining two are atoms or groups other than a hydrogen atom.

Among these, a methyl group or an isopropyl group is preferable as R^{S2}.

The group represented by Formula (S-4) is as follows.

*—C(═O)R^{Ac2} Formula (S-4)

R^{Ac2} represents a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 6 carbon atoms, which may have a halogen atom, or an aromatic ring group which may have a substituent selected from the substituent group R^{Ar1}.

As the aliphatic hydrocarbon group, an alkyl group is preferable.

In addition, as the aromatic ring group which may have a substituent selected from the substituent group R^{Ar1}, an aromatic ring group having 4 to 10 ring member atoms, which may have a linear aliphatic hydrocarbon group having 1 to 3 carbon atoms, a branched aliphatic hydrocarbon group having 3 to 5 carbon atoms, or a cyclic aliphatic hydrocarbon group having 3 to 8 carbon atoms is preferable, and a phenyl group which may have a linear aliphatic hydrocarbon group having 1 to 3 carbon atoms, a branched aliphatic hydrocarbon group having 3 to 5 carbon atoms, or a cyclic aliphatic hydrocarbon group having 3 to 8 carbon atoms is more preferable.

In Formula (1), Z¹ to Z⁶ each independently represent —CR^{X1}═ or a nitrogen atom. In a case where adjacent two of Z¹ to Z⁶ are —CR^{X1}═, two R^{X1}'s may be bonded to each other to form a ring.

R^{X1} represents a hydrogen atom or a substituent.

It is preferable that four or more of Z¹ to Z⁶ represent —CR^{X1}═, and it is more preferable that all of Z¹ to Z⁶ represent —CR^{X1}═.

In a case where two or more of Z¹ to Z⁶ represent —CR^{X1}═, R^{X1}'s may be the same as or different from each other.

Among these, it is preferable that two or less of Z¹ to Z⁶ (preferably, two or less of Z², Z³, Z⁵, and Z⁶) are —CR^{X1}═ where R^{X1} is a substituent, and the remains of Z¹ to Z⁶ are —CH═, and it is more preferable that all of Z¹ to Z⁶ are —CH═.

Examples of the substituent represented by R^{X1} include the groups exemplified by the substituent W, and more specific examples thereof include a halogen atom and an alkyl group.

As the halogen atom, a fluorine atom or a chlorine atom is preferable.

As the alkyl group, an alkyl group having 1 to 3 carbon atoms is preferable, a linear alkyl group having 1 to 3 carbon atoms is more preferable, and a methyl group is still more preferable.

In Formula (1), R¹ and R² each independently represent a hydrogen atom or a substituent.

Examples of the substituent represented by R¹ and R² include a group exemplified by the above-described substituent W. Among these, from the viewpoint of being more excellent in the effect of the present invention, it is preferable that R¹ and R² are each a hydrogen atom.

In Formula (1), A¹ and A² each independently represent a group represented by Formula (A-1).

In Formula (A-1), Y¹'s each independently represent a sulfur atom, an oxygen atom, ═NR^{X2}, or ═CR^{X3}R^{X4}. R^{X2} represents a hydrogen atom or a substituent. R^{X3} and R^{X4} each independently represent a cyano group, —SO₂R^{X5}, —COOR^{X6}, or —COR^{X7}.

From the viewpoint that the effect of the present invention is more excellent, Y¹ preferably represents an oxygen atom or a sulfur atom.

Examples of the substituent represented by R^{X2} include a substituent exemplified by the above-described substituent W.

In addition, R^{X5} to R^{X7} each independently represent an aliphatic hydrocarbon group which may have a substituent, an aromatic ring group which may have a substituent, or an aliphatic heterocyclic group which may have a substituent. Examples of the substituent which may be contained in the groups represented by R^{X5} to R^{X7} include the substituent exemplified by the substituent W.

The definition of the aliphatic hydrocarbon group is as described above, and among these, an alkyl group is preferable, and a linear alkyl group is more preferable. The number of carbon atoms in the aliphatic hydrocarbon group is preferably 1 to 3.

The definition of the aromatic ring group is as described above, and among these, an aryl group is preferable, and a phenyl group is more preferable.

The definition of the aliphatic heterocyclic group is as described above.

In Formula (A-1), C¹ represents a ring which contains two or more carbon atoms and may have a substituent.

The number of carbon atoms of the ring is preferably 3 to 30, more preferably 3 to 20, and still more preferably 3 to 10. The number of the carbon atoms is a number containing two carbon atoms specified in the formula.

The ring may be any of aromatic or non-aromatic.

The ring may be any of a monocyclic ring or a polycyclic ring, and is preferably a 5-membered ring, a 6-membered ring, or a fused ring including at least one of a 5-membered ring or a 6-membered ring. The number of rings forming the fused ring is preferably 1 to 4, and more preferably 1 to 3.

The ring may have a heteroatom. Examples of the heteroatom include a nitrogen atom, a sulfur atom, an oxygen atom, a selenium atom, a tellurium atom, a phosphorus atom, a silicon atom, and a boron atom, and a sulfur atom, a nitrogen atom, or an oxygen atom is preferable.

The number of heteroatoms in the ring is preferably 0 to 10 and more preferably 0 to 5.

Among the carbon atoms constituting the ring represented by C¹, a carbon atom other than the carbon atom at a bonding position to which * is attached in Formula (A-1) and the carbon atom bonded to Y¹ may be substituted with a carbonyl carbon (>C═O) or a thiocarbonyl carbon (>C═S).

Examples of the substituent which may be contained in the ring include a group exemplified by the above-described substituent W, and a halogen atom, an alkyl group, an aromatic ring group, or a silyl group is preferable and a halogen atom or an alkyl group is more preferable.

The alkyl group may be linear, branched, or cyclic, and is preferably linear.

The number of carbon atoms of the above-described alkyl group is preferably 1 to 10 and more preferably 1 to 3.

As the ring represented by C¹, a ring which is used as an acidic nucleus (for example, an acidic nucleus of a merocyanine coloring agent) is preferable, and examples thereof include the following nuclei:
(a) 1,3-dicarbonyl nuclei: for example, a 1,3-indandione nucleus, 1,3-cyclohexanedione, 5,5-dimethyl-1,3-cyclohexanedione, 1,3-dioxane-4,6-dione, and the like;
(b) pyrazolinone nuclei: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like;
(c) isoxazolinone nuclei: for example, 3-phenyl-2-isoxazolin-5-one, 3-methyl-2-isoxazolin-5-one, and the like;
(d) oxindole nuclei: for example, 1-alkyl-2,3-dihydro-2-oxindole and the like;
(e) 2,4,6-trioxohexahydropyrimidine nuclei: for example, barbituric acid, 2-thibarbituric acid and derivatives thereof, and the like, where examples of the derivatives include 1-alkyl compounds such as 1-methyl and 1-ethyl, 1,3-dialkyl compounds such as 1,3-dimethyl, 1,3-diethyl, and 1,3-dibutyl, 1,3-diaryl compounds such as 1,3-diphenyl, 1,3-di(p-chlorophenyl), and 1,3-di(p-ethoxycarbonylphenyl), 1-alkyl-1-aryl compounds such as 1-ethyl-3-phenyl, and 1,3-diheteroaryl compounds such as 1,3-di(2-pyridyl);
(f) 2-thio-2,4-thiazolidinedione nuclei: for example, rhodanine and derivatives thereof, and the like, where examples of the derivatives include 3-aklylrhodanine such as 3-methylrhodanine, 3-ethylrhodanine, and 3-allylrhodanine, 3-arylrhodanine such as 3-phenylrhodanine, 3-heteroarylrhodanine such as 3-(2-pyridyl)rhodanine, and the like;
(g) 2-thio-2,4-oxazolidinedione nuclei (2-thio-2,4-(3H,5H)-oxazoledione nuclei): for example, 3-ethyl-2-thio-2,4-oxazolidinedione, and the like;
(h) thianaphthenone nuclei: for example, 3(2H)-thianaphthenone-1,1-dioxide, and the like;
(i) 2-thio-2,5-thiazolidinedione nuclei: for example, 3-ethyl-2-thio-2,5-thiazolidinedione, and the like;
(j) 2,4-thiazolidinedione nuclei: for example, 2,4-thiazolidinedione, 3-ethyl-2,4-thiazolidinedione, 3-phenyl-2,4-thiazolidinedione, and the like;
(k) thiazoliin-4-one nuclei: for example, 4-thiazolinone, 2-ethyl-4-thiazolinone, and the like;
(l) 2,4-imidazolidinedione (hydantoin) nuclei: for example, 2,4-imidazolidinedione, 3-ethyl-2,4-imidazolidinedione, and the like;
(m) 2-thio-2,4-imidazolidinedione (2-thiohydantoin) nuclei: for example, 2-thio-2,4-imidazolidinedione, 3-ethyl-2-thio-2,4-imidazolidinedione, and the like;
(n) imidazolin-5-one nuclei: for example, 2-propylmercapto-2-imidazolin-5-one, and the like;
(o) 3,5-pyrazolidinedione nuclei: for example, 1,2-diphenyl-3,5-pyrazolidinedione, 1,2-dimethyl-3,5-pyrazolidinedione, and the like;
(p) benzothiophen-3(2H)-one nuclei: for example, benzothiophen-3(2H)-one, oxobenzothiophen-3(2H)-one, dioxobenzothiophen-3(2H)-one, and the like;
(q) indanone nuclei: for example, 1-indanone, 3-phenyl-1-indanone, 3-methyl-1-indanone, 3,3-diphenyl-1-indanone, 3,3-dimethyl-1-indanone, and the like;
(r) benzofuran-3-(2H)-one nucleus: for example, benzofuran-3-(2H)-one, and the like; and
(s) 2,2-dihydrophenalene-1,3-dione nucleus, and the like.

From the viewpoint that the effect of the present invention is more excellent, the group represented by Formula (A-1) is preferably a group represented by Formula (A-2).

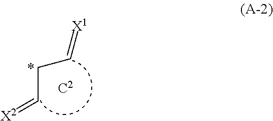

In Formula (A-2), X¹ and X² each independently represent an oxygen atom or a sulfur atom. It is preferable that both X¹ and X² represent an oxygen atom.

In addition, in Formula (A-2), C² represents a ring containing 3 or more carbon atoms.

Three carbon atoms included in C² are the three carbon atoms specified in Formula (A-2).

The number of carbon atoms of the ring is preferably 3 to 30, more preferably 3 to 20, and still more preferably 3 to 10. The number of carbon atoms in the ring is the number including three carbon atoms specified in the formula.

The ring may be any of an aromatic ring or a non-aromatic ring.

The ring may be any of a monocyclic ring or a polycyclic ring, and is preferably a 5-membered ring, a 6-membered ring, or a fused ring including at least one of a 5-membered ring or a 6-membered ring. In a case where the ring is polycyclic, the number of rings contained is preferably 2 to 6 and more preferably 2 or 3.

The ring may have a heteroatom. Examples of the heteroatom include a nitrogen atom, a sulfur atom, an oxygen atom, a selenium atom, a tellurium atom, a phosphorus atom, a silicon atom, and a boron atom, and a sulfur atom, a nitrogen atom, or an oxygen atom is preferable.

The number of heteroatoms contained in the ring is preferably 0 to 10 and more preferably 0 to 5.

Among the carbon atoms constituting the ring represented by C², a carbon atom other than the carbon atom at a bonding position to which * is attached in Formula (A-2) and the carbon atom bonded to X¹ and X² may be substituted with a carbonyl carbon (>C═O) or a thiocarbonyl carbon (>C═S).

The suitable aspect of the substituent which may be contained in the ring is the same as that of the substituent which may be contained in the above-described ring C¹.

In addition, the group represented by Formula (A-2) is preferably a group represented by Formula (C-1) or a group represented by Formula (C-2).

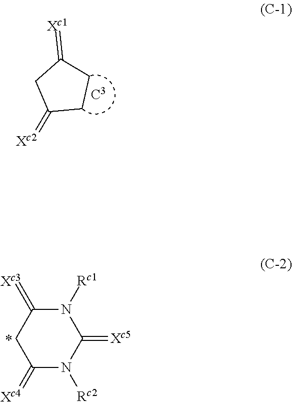

in Formula (C-1), X^{c1} and X^{c2} each independently represent a sulfur atom or an oxygen atom.

It is preferable that at least one of X^{c1} or X^{c2} is an oxygen atom, and it is more preferable that both X^{c1} and X^{c2} are oxygen atoms.

In Formula (C-1), C³ represents an aromatic ring which may have a substituent.

The number of carbon atoms of the aromatic ring group is preferably 4 to 30, more preferably 5 to 12, and still more preferably 6 to 8. The number of the carbon atoms is a number containing two carbon atoms specified in the formula.

The aromatic ring group may be any of a monocyclic ring or a polycyclic ring.

In addition, the aromatic ring may be any of an aromatic hydrocarbon ring or an aromatic heterocyclic ring, and an aromatic hydrocarbon ring is preferable.

Examples of the aromatic ring represented by C³ include the ring exemplified in the description of the above-described aromatic ring.

Among these, as the aromatic ring represented by C³, a benzene ring, a naphthalene ring, an anthracene ring, or a pyrene ring is preferable, and a benzene ring is more preferable.

Examples of the substituent which may be included in the above-described aromatic ring include the group exemplified by the above-described substituent W.

In Formula (C-2), X^{c3} to X^{c5} represent a sulfur atom or an oxygen atom.

It is preferable that all of X^{c3} to X^{c5} are oxygen atoms.

In addition, R^{c1} and R^{c2} each independently represent a hydrogen atom or a substituent. Examples of the substituent represented by R^{c1} and R^{c2} include the group exemplified by the above-described substituent W, and among these, an alkyl group or a phenyl group is preferable, and an alkyl group is more preferable.

The above-described phenyl group may further have a substituent, and examples thereof include a group exemplified by the above-described substituent W.

In Formula (1), in a case where A¹ and A² are each a group represented by Formula (A-1), the specific compound is represented by Formula (1A-1), and in a case where A¹ and A² are each a group represented by Formula (A-2), the specific compound is represented by Formula (1A-2).

In addition, in a case where the group represented by Formula (A-2) is a group represented by Formula (C-1), the specific compound is represented by Formula (1C-1), and in a case where the group represented by Formula (A-2) is a group represented by Formula (C-2), the specific compound is represented by Formula (1C-2).

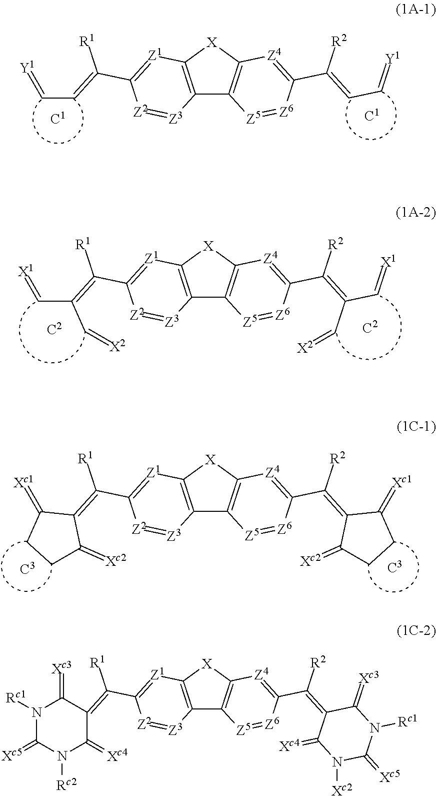

A molecular weight of the specific compound is preferably 400 to 1,200, more preferably 400 to 1,000, and still more preferably 500 to 800.

In a case where the molecular weight is in the above-described range, it is presumed that the sublimation temperature of the specific compound becomes low, and the quantum efficiency is excellent also in a case where a photoelectric conversion film is formed at a high speed.

In the specific compound, an ionization potential in a single film is preferably −6.0 to −5.0 eV from the viewpoints of stability in a case of using the compound as the p-type organic semiconductor and matching of energy levels between the compound and the n-type organic semiconductor.

The maximal absorption wavelength of the specific compound is preferably in a wavelength range of 400 to 600 nm, and more preferably in a wavelength range of 400 to 500 nm.

The maximal absorption wavelength is a value measured in a solution state (solvent: chloroform) by an absorption spectrum of the specific compound being adjusted to a concentration having an absorbance of about 0.5 to 1.0. Provided that in a case where the specific compound is not soluble in chloroform, a value measured by using the specific compound in which the specific compound is vapor-deposited and formed into a film state is defined as a maximal absorption wavelength of the specific compound.

The specific compound is particularly useful as a material of the photoelectric conversion film used for the imaging element, the optical sensor, or a photoelectric cell. The specific compound often functions as a coloring agent in the photoelectric conversion film. The specific compound can also be used as a coloring material, a liquid crystal material, an organic semiconductor material, a charge transport material, a pharmaceutical material, and a fluorescent diagnostic material.

Specific examples of the specific compound are shown below, but the present invention is not limited thereto.

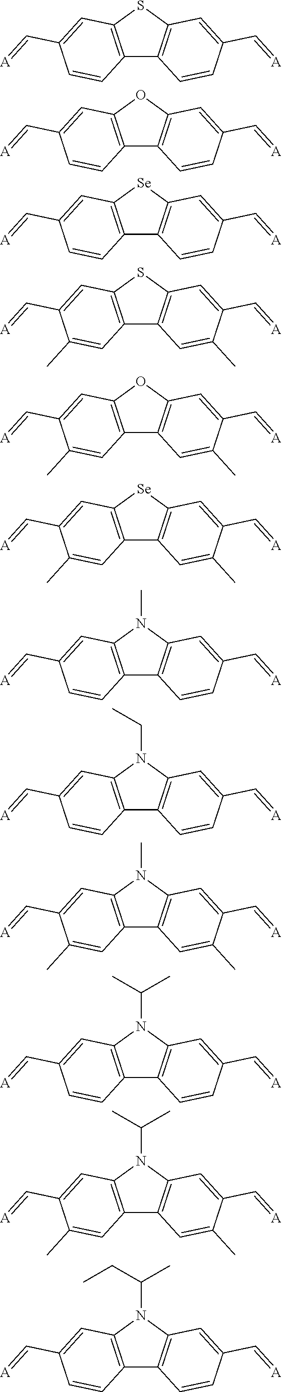

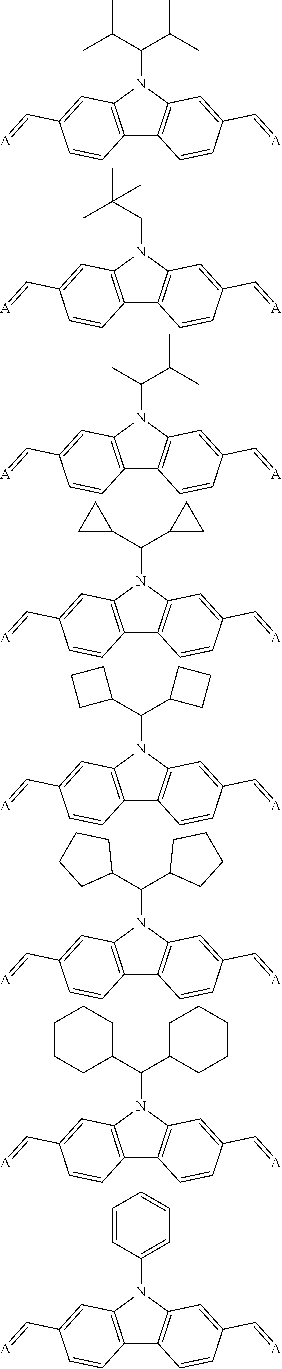

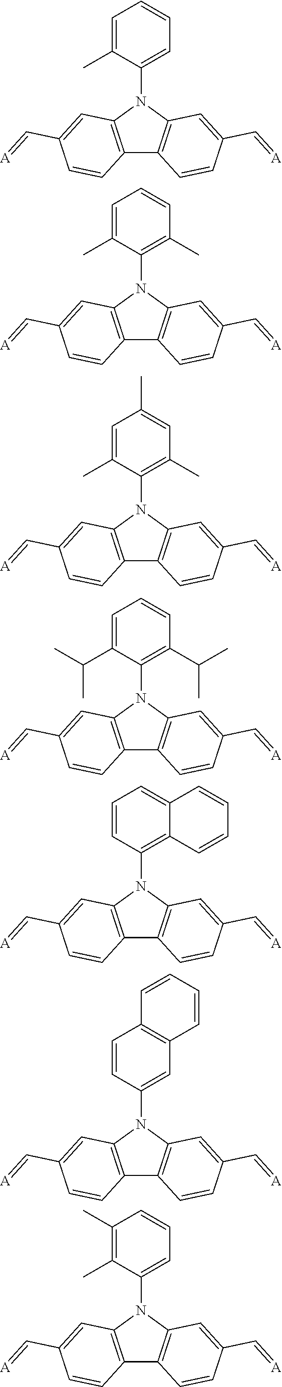

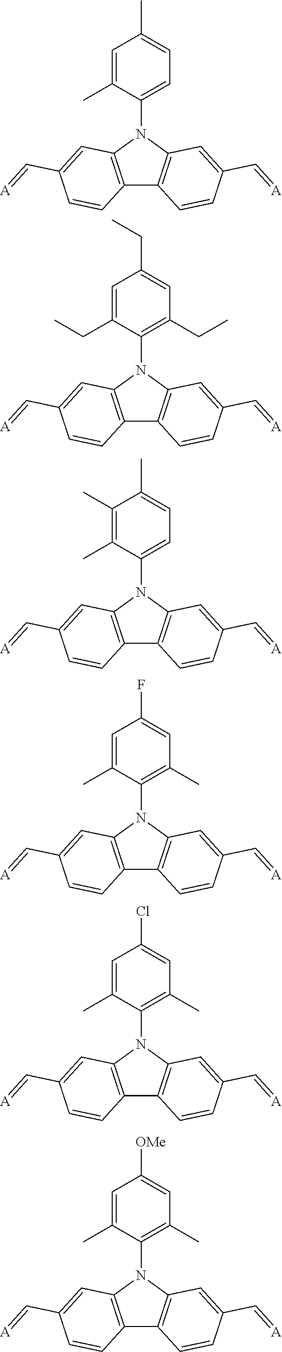

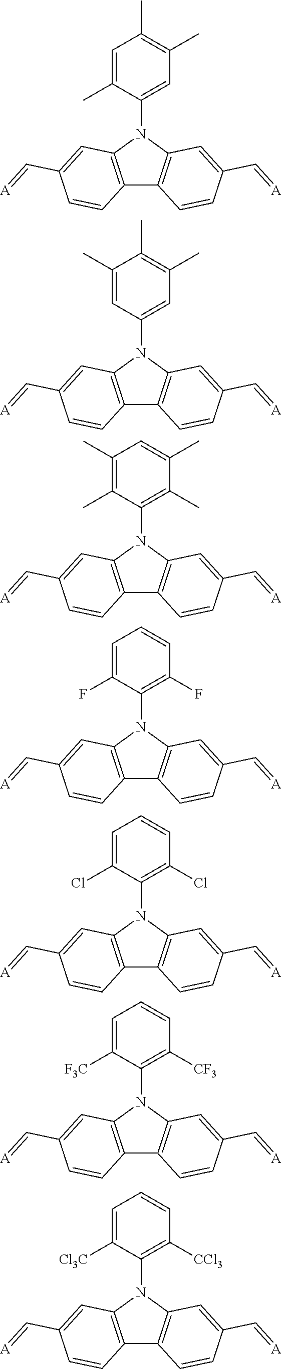

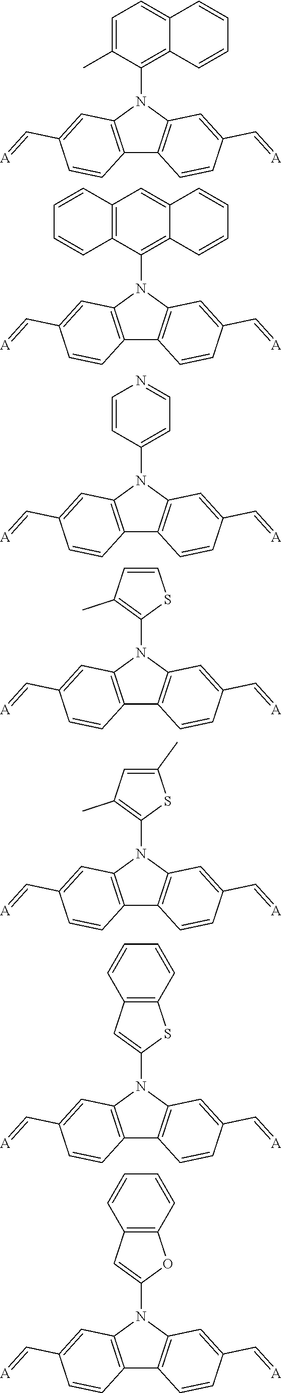

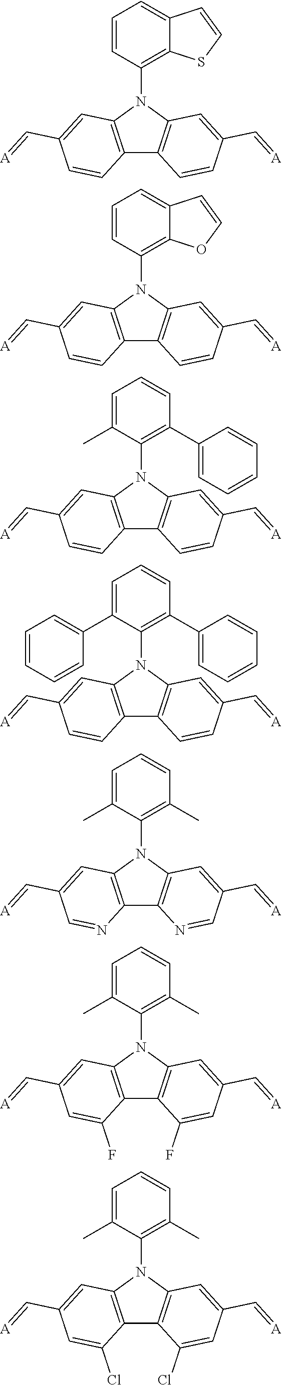

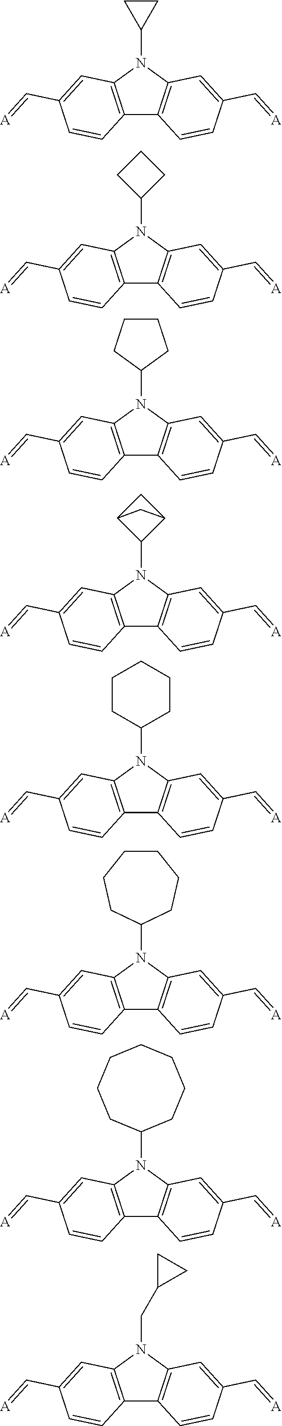

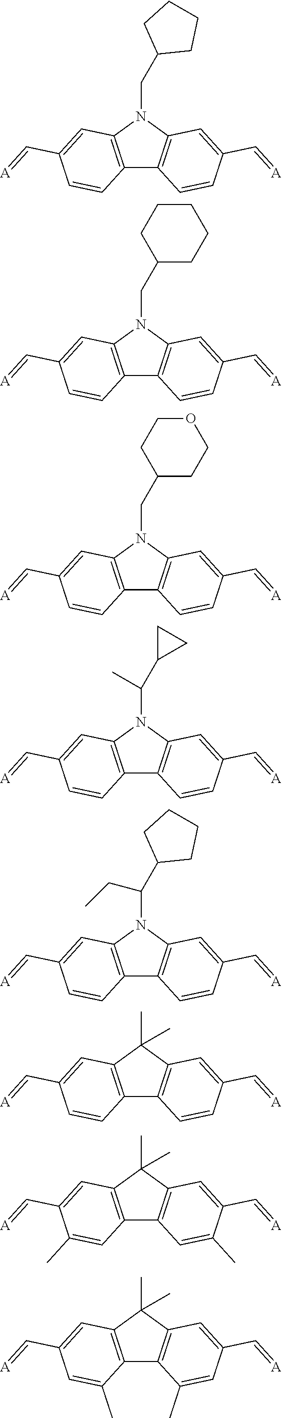

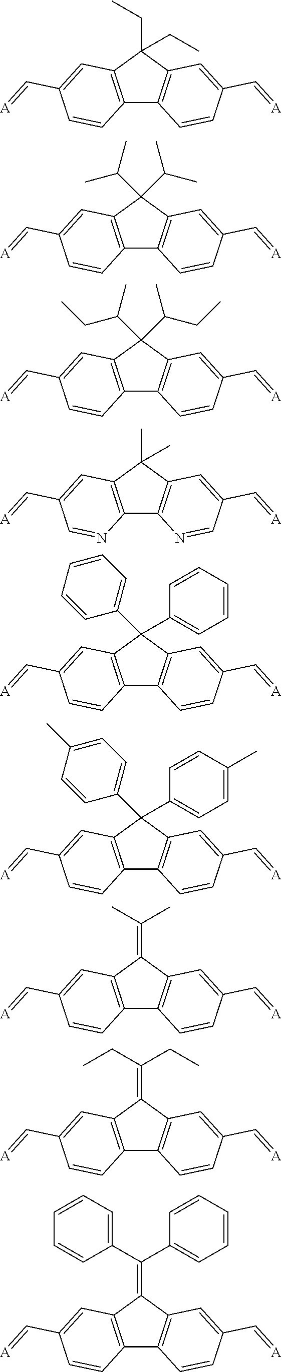

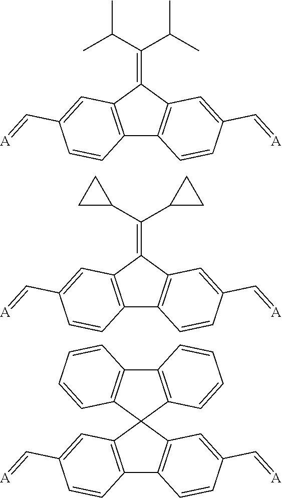

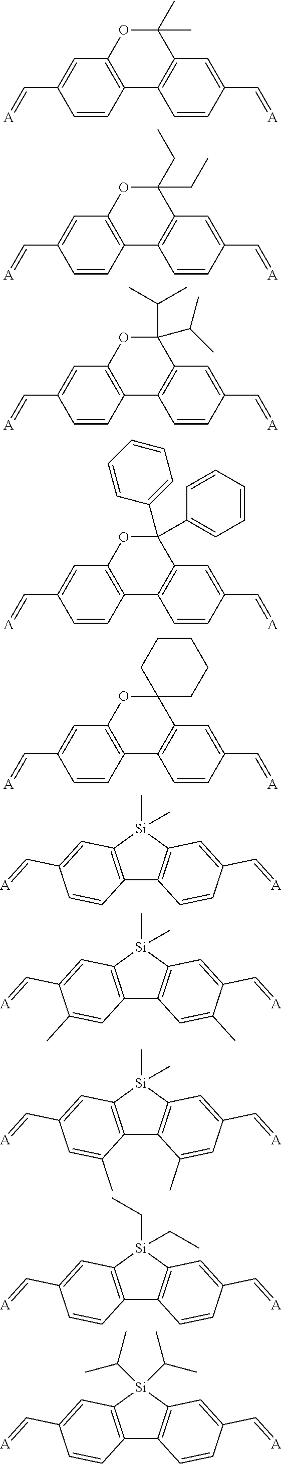

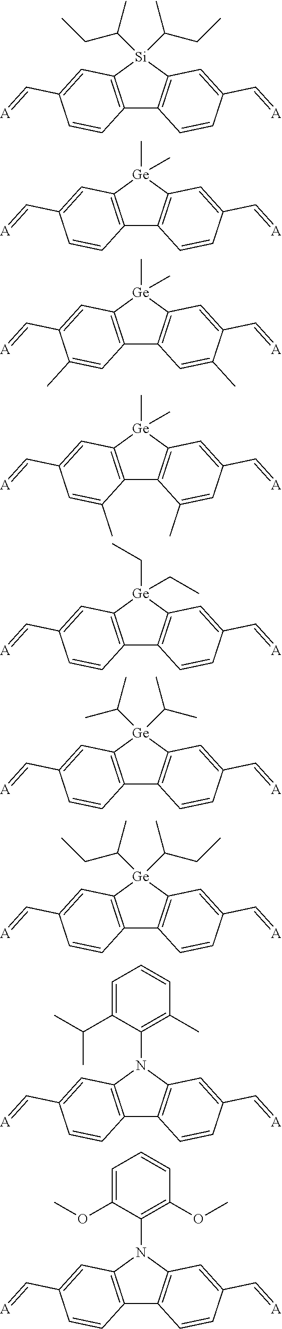

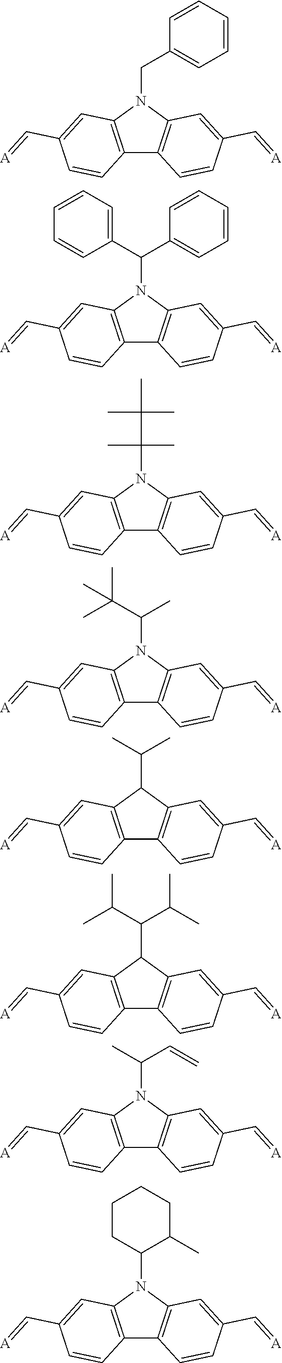

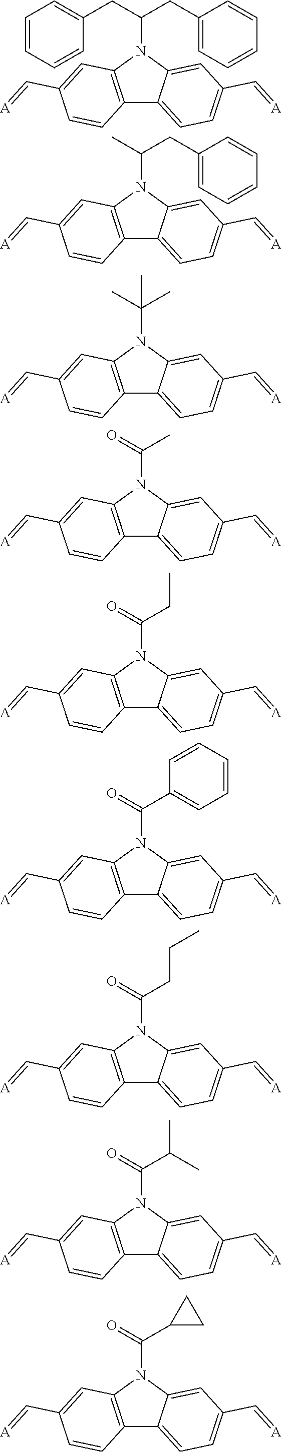

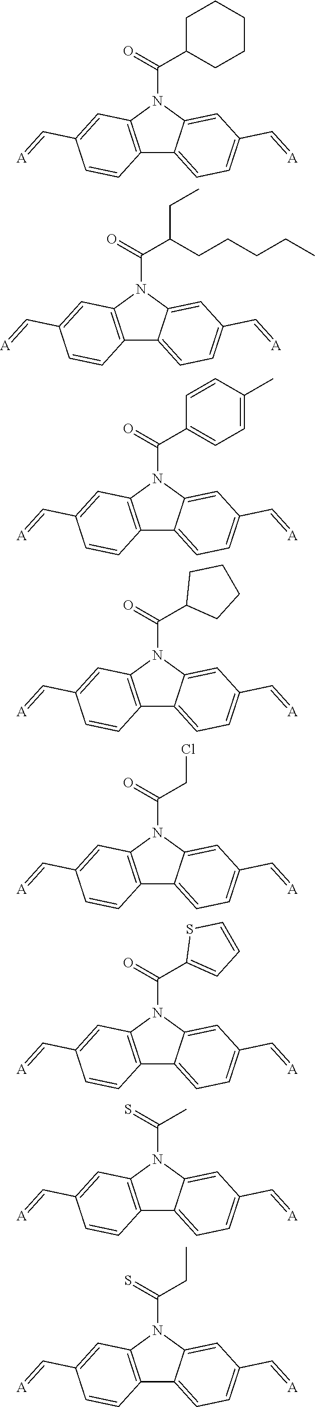

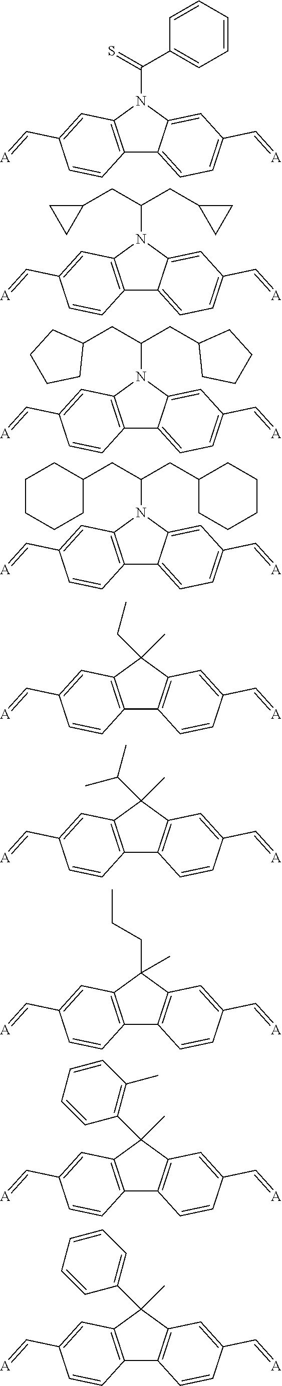

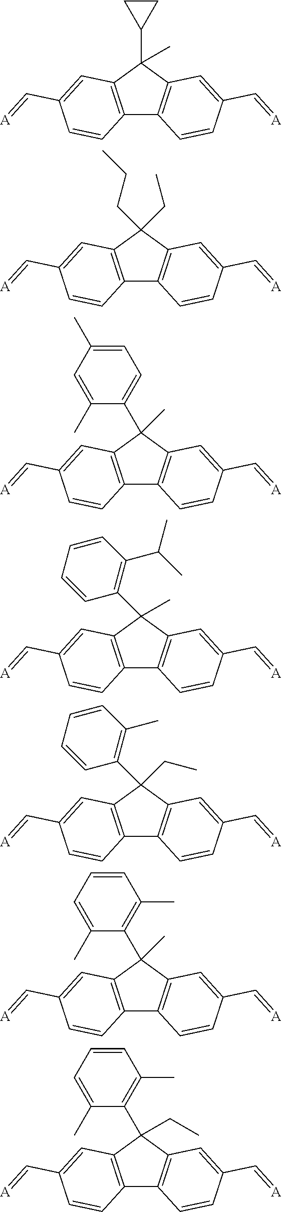

A in the specific compound exemplified above is represented by any of the following groups.

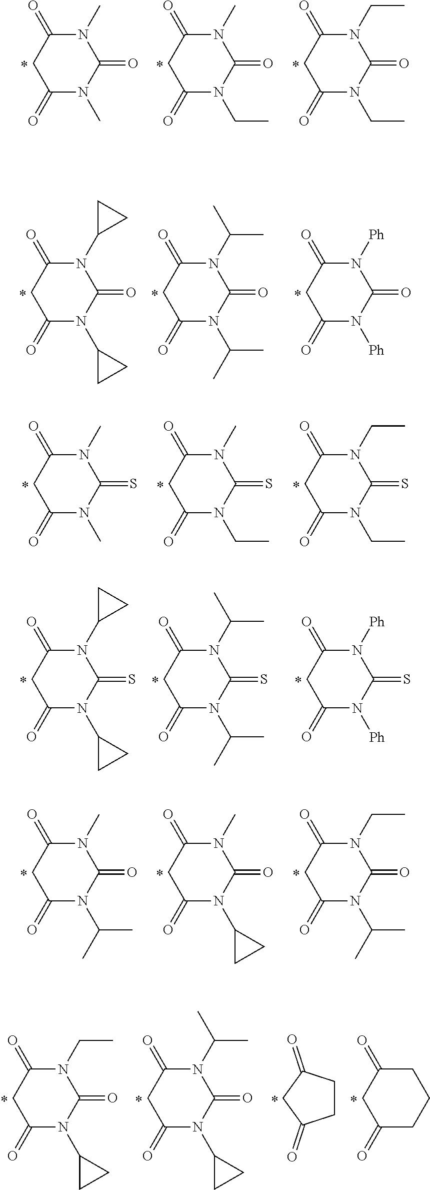

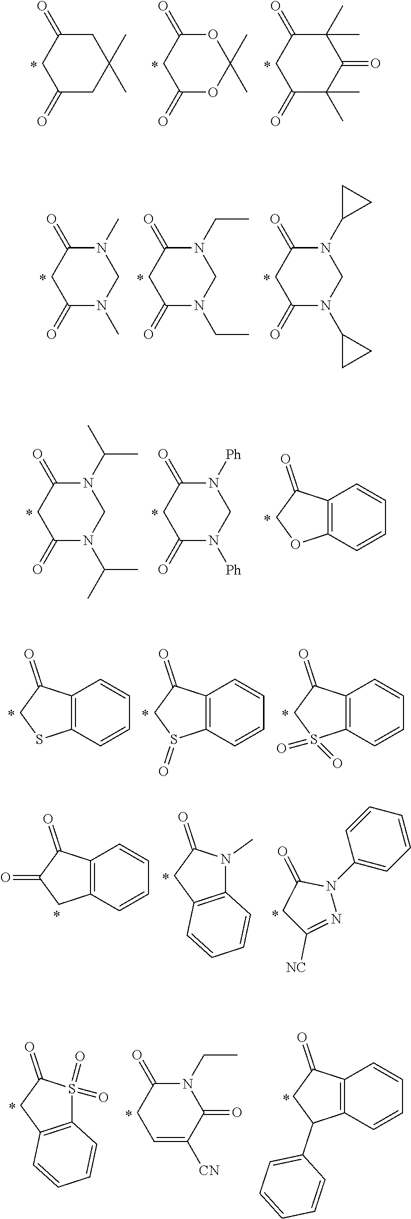

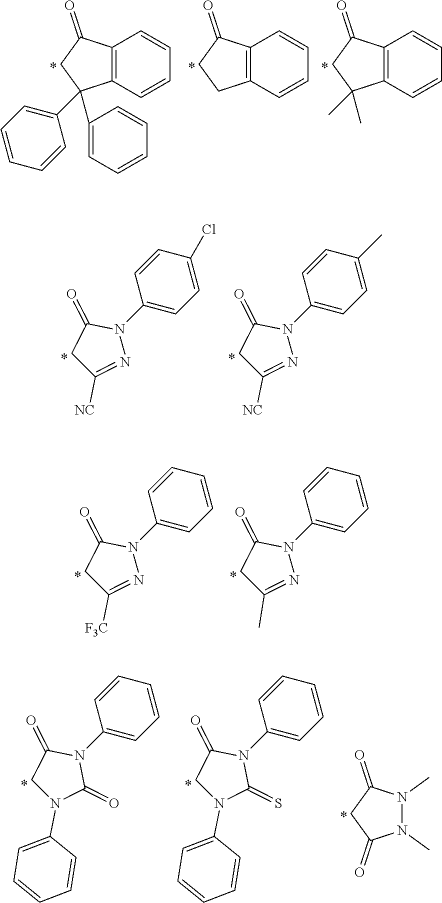

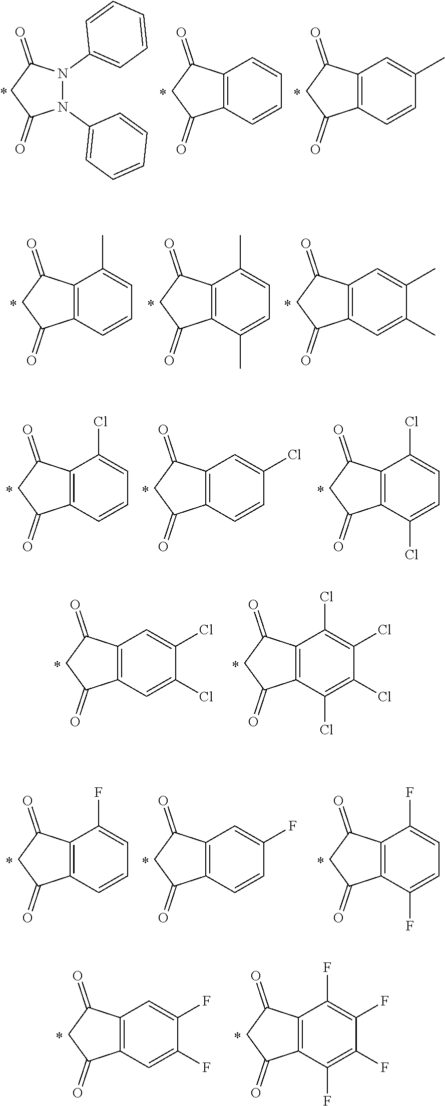

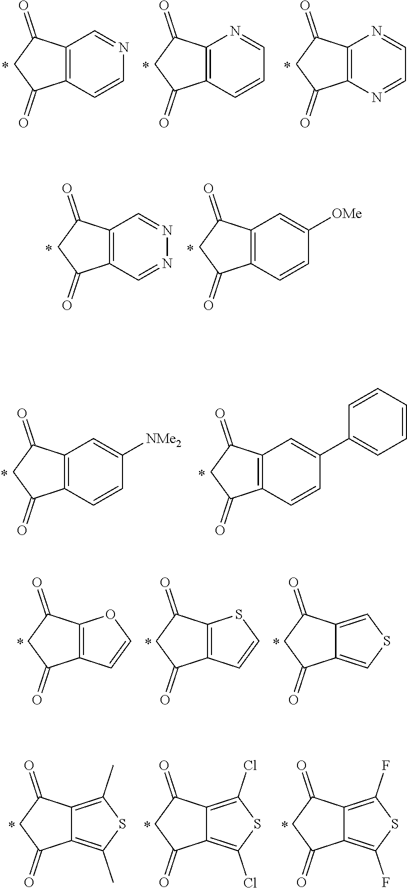

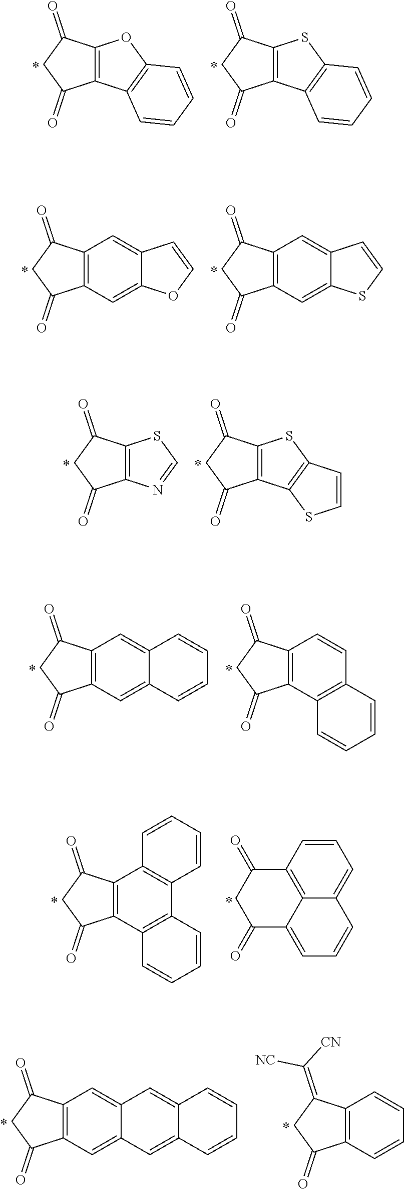

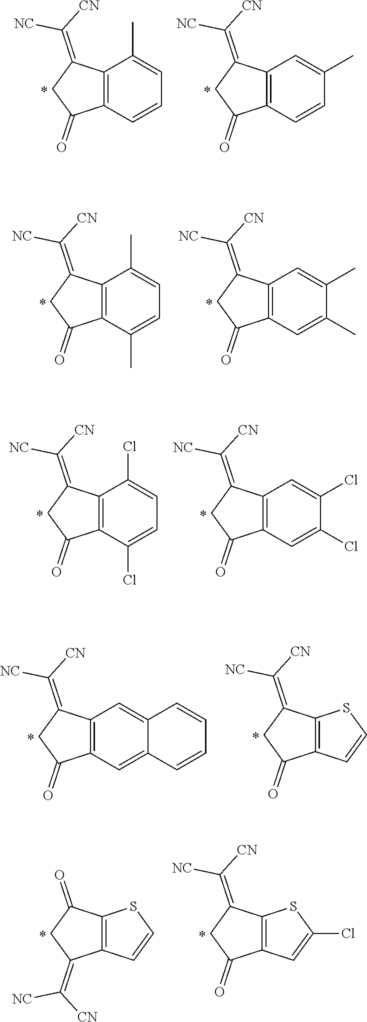

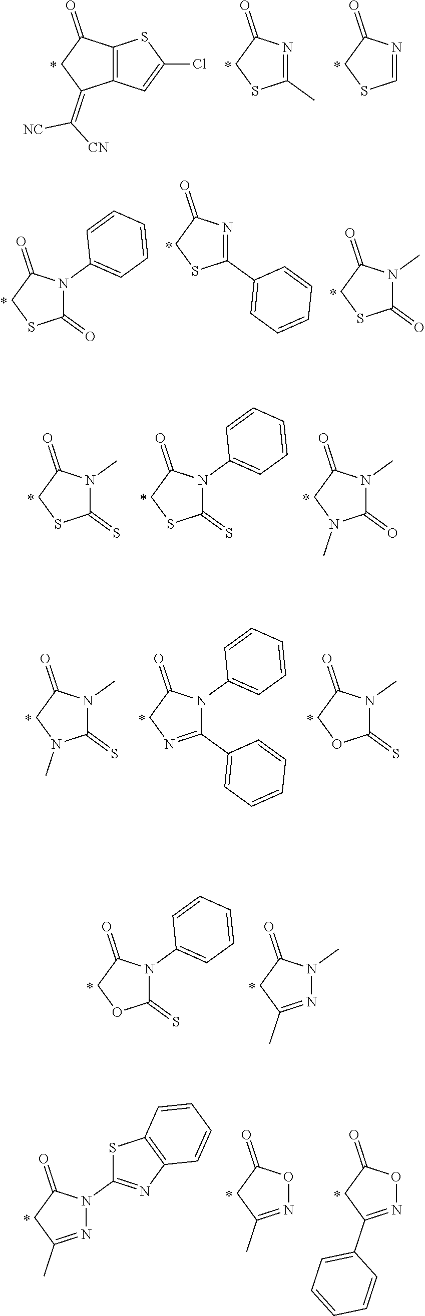

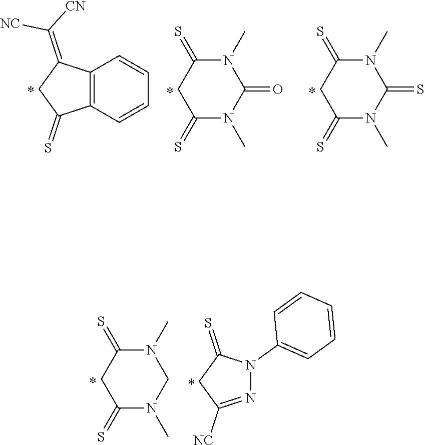

The specific compound may be purified as necessary.

Examples of a purification method of the specific compound include sublimation purification, purification using silica gel column chromatography, purification using gel permeation chromatography, reslurry washing, repurification by reprecipitation, purification using an adsorbent such as activated carbon, and recrystallization purification.

A content of the specific compound in the photoelectric conversion film (=film thickness of specific compound in terms of single layer/film thickness of photoelectric conversion film×100) is not particularly limited, and is preferably 15% to 75% by volume, more preferably 20% to 60% by volume, and still more preferably 20% to 50% by volume.

The specific compound may be used alone or in combination of two or more types thereof. In a case where two or more types thereof are used, the total amount thereof is preferably within the above-described range.

<n-Type Organic Semiconductor>

The photoelectric conversion film preferably contains the n-type organic semiconductor in addition to the specific compound.

The n-type organic semiconductor is a compound different from the specific compound.

The n-type organic semiconductor is an acceptor-property organic semiconductor material (a compound), and refers to an organic compound having a property of easily accepting an electron. That is, the n-type organic semiconductor refers to an organic compound having a large electron affinity of two organic compounds used in contact with each other. That is, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative and oxadiazole derivatives; anthraquinodimethane derivatives; diphenylquinone derivatives; bathocuproine, bathophenanthroline, and derivatives thereof, triazole compounds; a distyrylarylene derivative; a metal complex having a nitrogen-containing heterocyclic compound as a ligand; a silole compound; and compounds disclosed in paragraphs [0056] to [0057] of JP2006-100767A.

The n-type organic semiconductor (compound) is preferably fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1,200, and more preferably 200 to 900.

The maximal absorption wavelength of the n-type organic semiconductor is preferably in a wavelength of 400 nm or less or in a wavelength range of 500 to 600 nm.

It is preferable that the photoelectric conversion film has a bulk hetero structure formed in a state in which the specific compound and the n-type organic semiconductor are mixed. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A.

The difference in electron affinity between the specific compound and the n-type organic semiconductor is preferably 0.1 eV or more.

The n-type organic semiconductor may be used alone, or two or more types thereof may be used in combination.

In a case where the photoelectric conversion film contains the n-type organic semiconductor, a content of the n-type organic semiconductor in the photoelectric conversion film (film thickness of n-type organic semiconductor in terms of single layer/film thickness of photoelectric conversion film×100) is preferably 15% to 75% by volume, more preferably 20% to 60% by volume, and still more preferably 20% to 50% by volume.

In a case where the n-type organic semiconductor material includes fullerenes, a content of the fullerenes to a total content of the n-type organic semiconductor material (film thickness of fullerenes in terms of single layer/total film thickness of n-type organic semiconductor materials in terms of single layer×100) is preferably 50% to 100% by volume, and more preferably 80% to 100% by volume. The fullerenes may be used alone, or two or more types thereof may be used in combination.

From the viewpoint of response speed of the photoelectric conversion element, the content of the specific compound to the total content of the specific compound and the n-type organic semiconductor (film thickness in terms of single layer of specific compound/(film thickness in terms of single layer of specific compound+film thickness in terms of single layer of n-type organic semiconductor)×100) is preferably 20% to 80% by volume, and more preferably 40% to 80% by volume.

In a case where the photoelectric conversion film contains an n-type organic semiconductor and a p-type organic semiconductor, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor)×100) is preferably 15% to 75% by volume, and more preferably 30% to 75% by volume.

It is preferable that the photoelectric conversion film is substantially formed of the specific compound, the n-type organic semiconductor, and the p-type organic semiconductor included as desired. The term “substantially” indicates that the total content of the specific compound, the n-type organic semiconductor, and the p-type organic semiconductor is 90% to 100% by volume, preferably 95% to 100% by volume, and more preferably 99% to 100% by volume, with respect to the total mass of the photoelectric conversion film.

<p-Type Organic Semiconductor>

The photoelectric conversion film preferably contains the p-type organic semiconductor in addition to the specific compound.

The p-type organic semiconductor is a compound different from the specific compound.

The p-type organic semiconductor is a donor organic semiconductor material (a compound), and refers to an organic compound having a property of easily donating an electron. That is, the p-type organic semiconductor means an organic compound having a smaller ionization potential in a case where two organic compounds are used in contact with each other.

The p-type organic semiconductor may be used alone, or two or more types thereof may be used in combination.

Examples of the p-type organic semiconductor include triarylamine compounds (for example, N,N′-bis(3-methylphenyl)-(1,1′-biphenyl)-4,4′-diamine (TPD), 4,4′-bis[N-(naphthyl)-N-Phenyl-amino]biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-094660A), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno [3,2-f: 4,5-f] bis [1]benzothiophene (TBBT) derivative, compounds disclosed in paragraphs [0031] to [0036] of JP2018-014474A, compounds disclosed in paragraphs [0043] to [0045] of WO2016/194630A, compounds disclosed in paragraphs [0025] to [0037], and [0099] to [0109] of WO2017/159684A, compounds disclosed in paragraphs [0029] to [0034] of JP2017-076766A, compounds disclosed in paragraphs [0015] to [0025] of WO2018/207722A, compounds disclosed in paragraphs [0045] to [0053] of JP2019-054228A, compounds disclosed in paragraphs [0045] to [0055] of WO2019/058995A, compounds disclosed in paragraphs [0063] to [0089] of WO2019/081416A, compounds disclosed in paragraphs [0033] to [0036] of JP2019-80052A, compounds disclosed in paragraphs [0044] to [0054] of WO2019/054125A, compounds disclosed in paragraphs [0041] to [0046] of WO2019/093188A), compounds in paragraphs [0034] to [0037] of JP2019-050398A, compounds in paragraphs [0033] to [0036] of JP2018-206878A, compounds in paragraph [0038] of JP2018-190755A, compounds in paragraphs [0019] to [0021] of JP2018-026559A, compounds in paragraphs [0031] to [0056] of JP2018-170487A, compounds in paragraphs [0036] to [0041] of JP2018-078270A, compounds in paragraphs [0055] to [0082] of JP2018-166200A, compounds in paragraphs [0041] to [0050] of JP2018-113425A, compounds in paragraphs [0044] to [0048] of JP2018-085430A, compounds in paragraphs [0041] to [0045] of JP2018-056546A, compounds in paragraphs [0042] to [0049] of JP2018-046267A, compounds in paragraphs [0031] to [0036] of JP2018-014474A, compounds disclosed in paragraphs [0036] to [0046] of WO2018/016465A, compounds disclosed in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

In addition, examples of the p-type organic semiconductor also include a benzoxazole compound (for example, compounds described in FIGS. 3 to 7 of JP2022-123944A), a dicarbazole compound (for example, compounds described in FIGS. 2 to 5 of JP2022-122839A), a benzoquinazoline compound (for example, compounds described in paragraphs [0053] to [0056] of JP2022-120323A), an azine compound (for example, compounds described in paragraphs [0041] and [0042] of JP2022-120273A), compounds described in FIGS. 2 to 10 of JP2022-115832A, an indolotriphenylene compound (for example, compounds described in paragraphs [0065] to [0072] of JP2022-108268A), an indolocarbazole compound (for example, compounds described in paragraphs [0052] to [0073] of JP2023-005703A and paragraph [0028] of JP2022-100258A), a triscarbazolylphenyl compound (for example, compounds described in paragraphs [0038] to [0040] of JP2022-181226A), compounds described in paragraphs [0070] to [0082] of JP2022-027575A, compounds described in paragraphs [0051] to [0064] of JP2021-163968A, and the like.

Examples of the p-type organic semiconductor also include compounds having an ionization potential smaller than that of the n-type organic semiconductor, and in a case where this condition is satisfied, the organic coloring agents exemplified as the n-type organic semiconductor can be used.

The compounds that can be used as the p-type organic semiconductor compound are exemplified below.

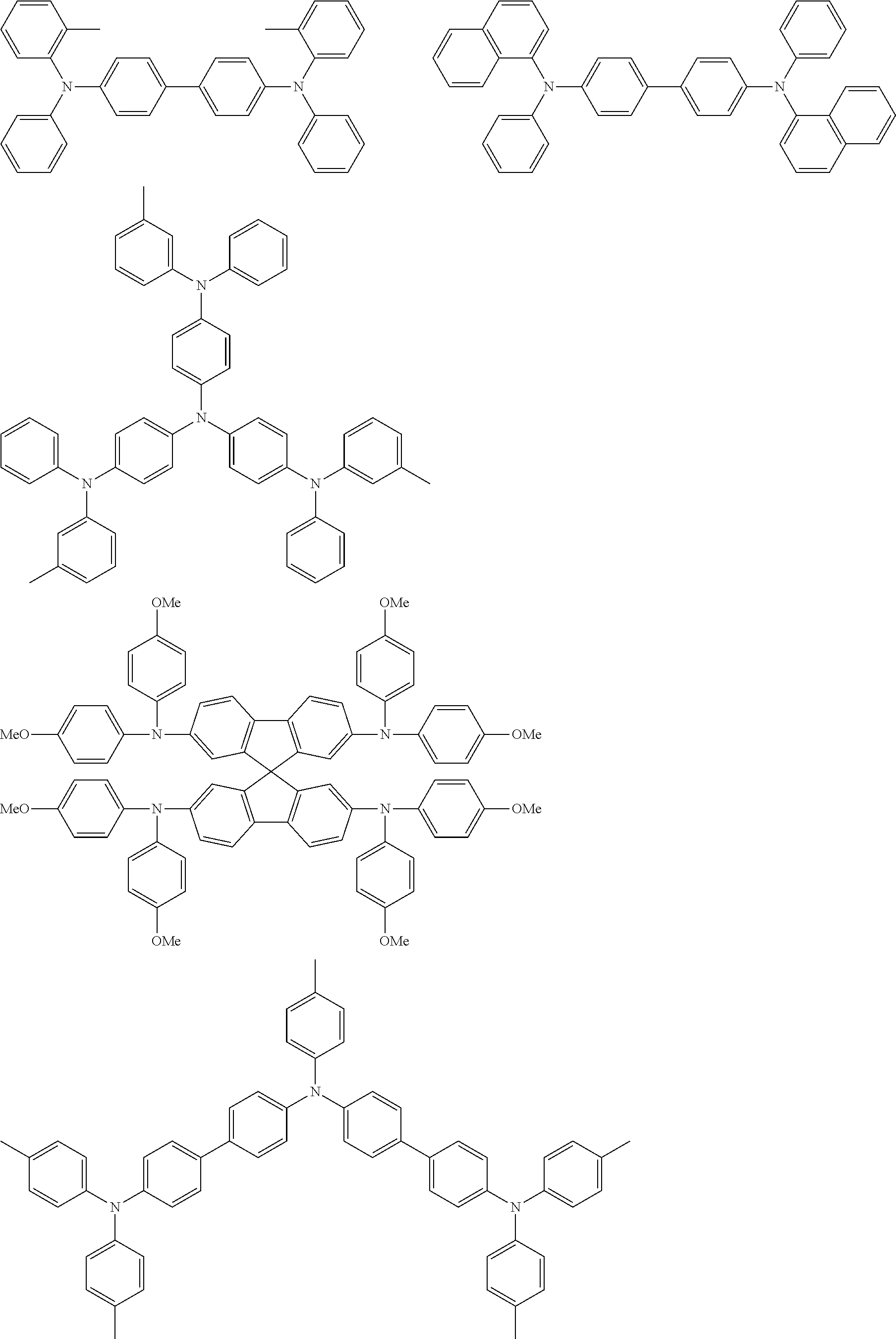

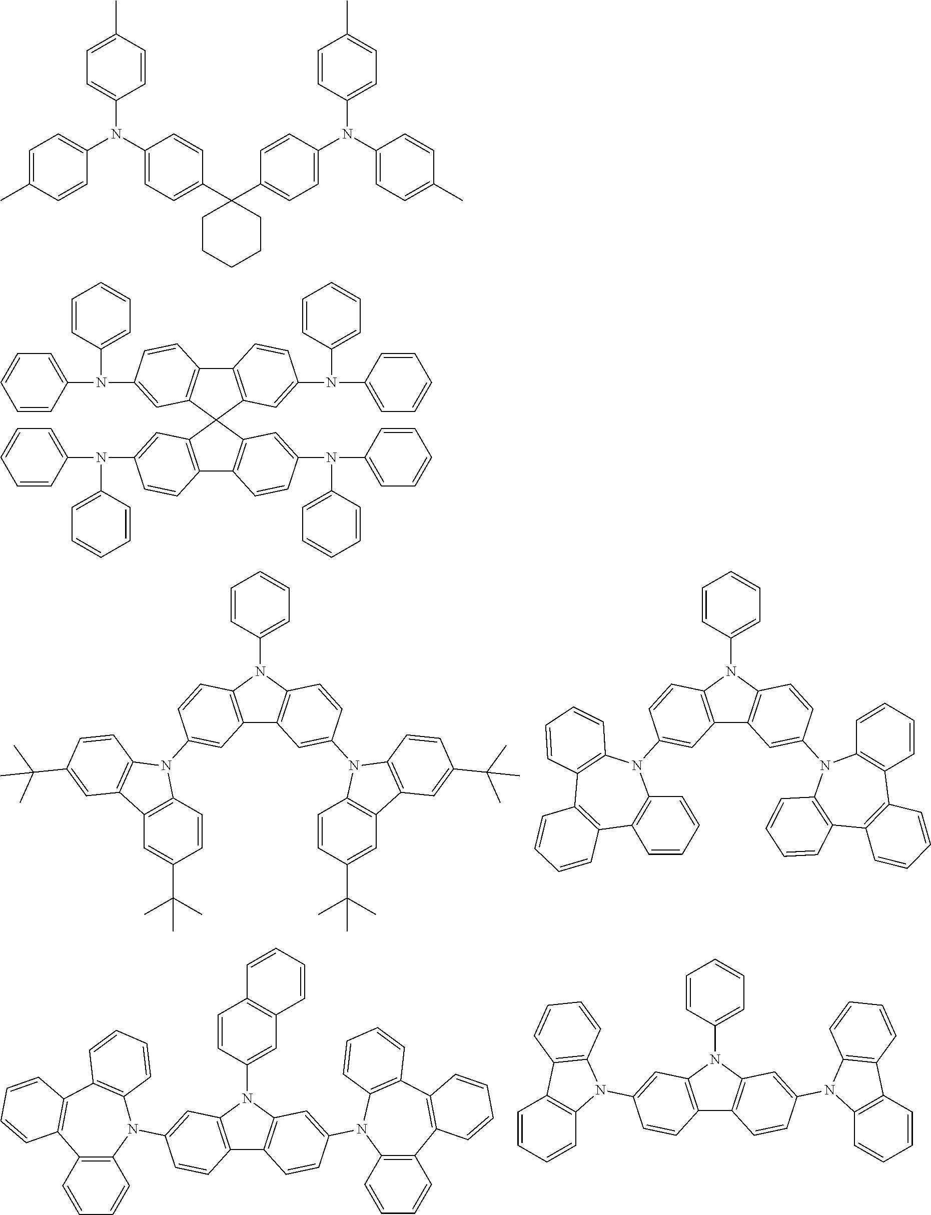

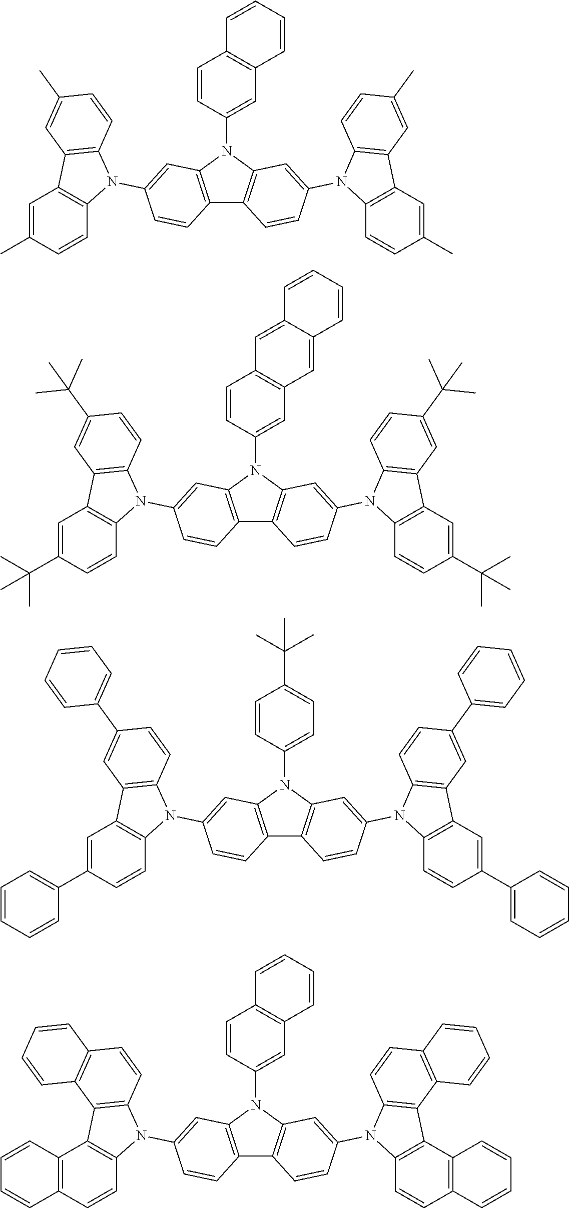

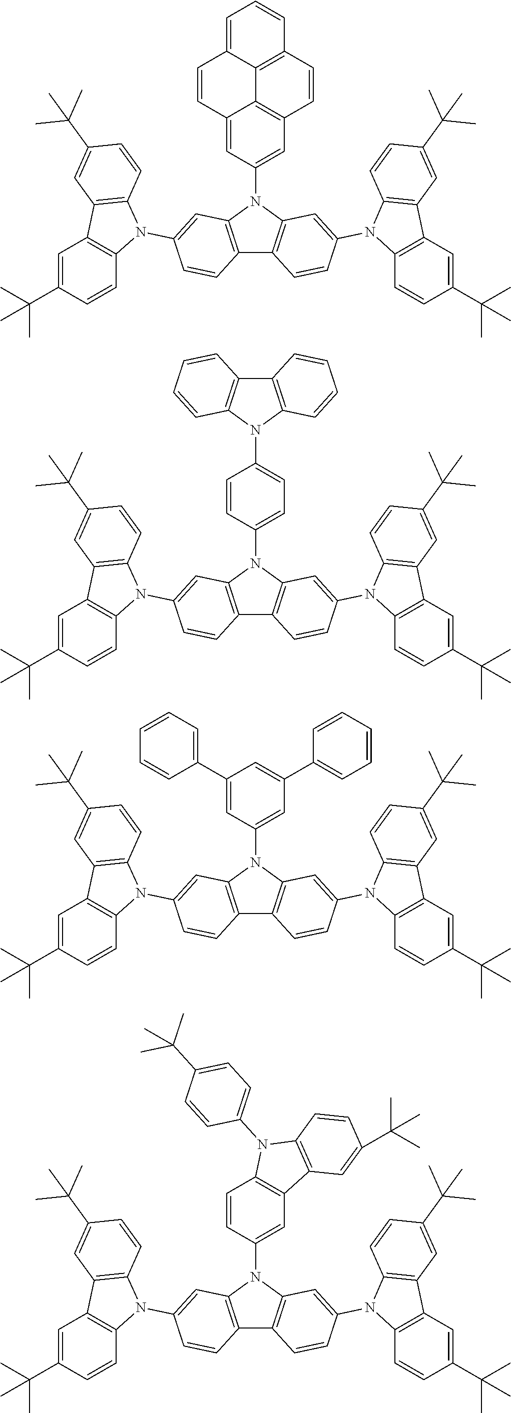

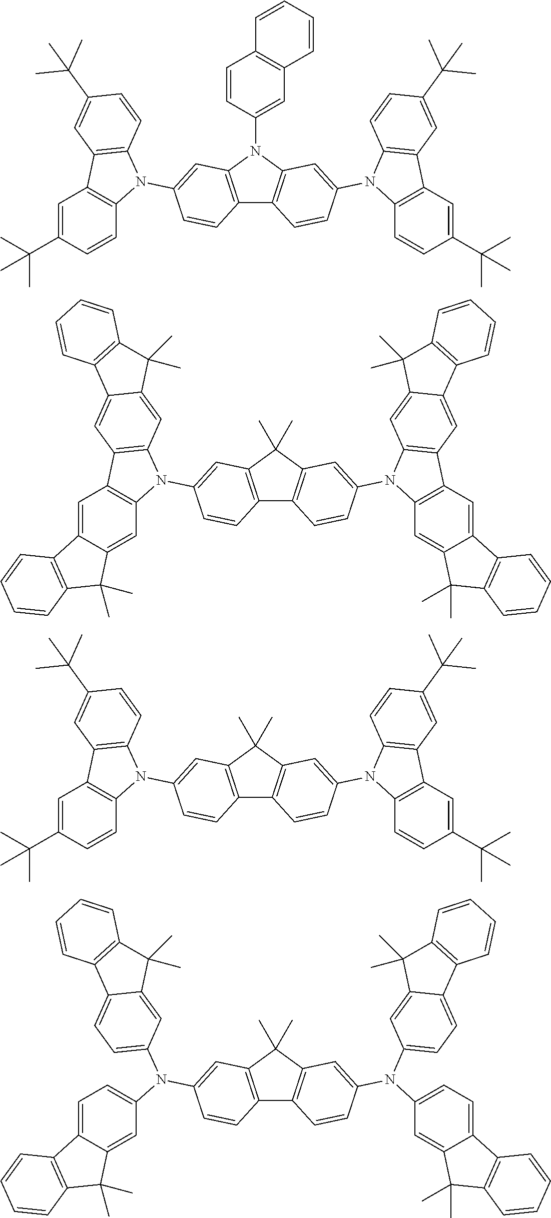

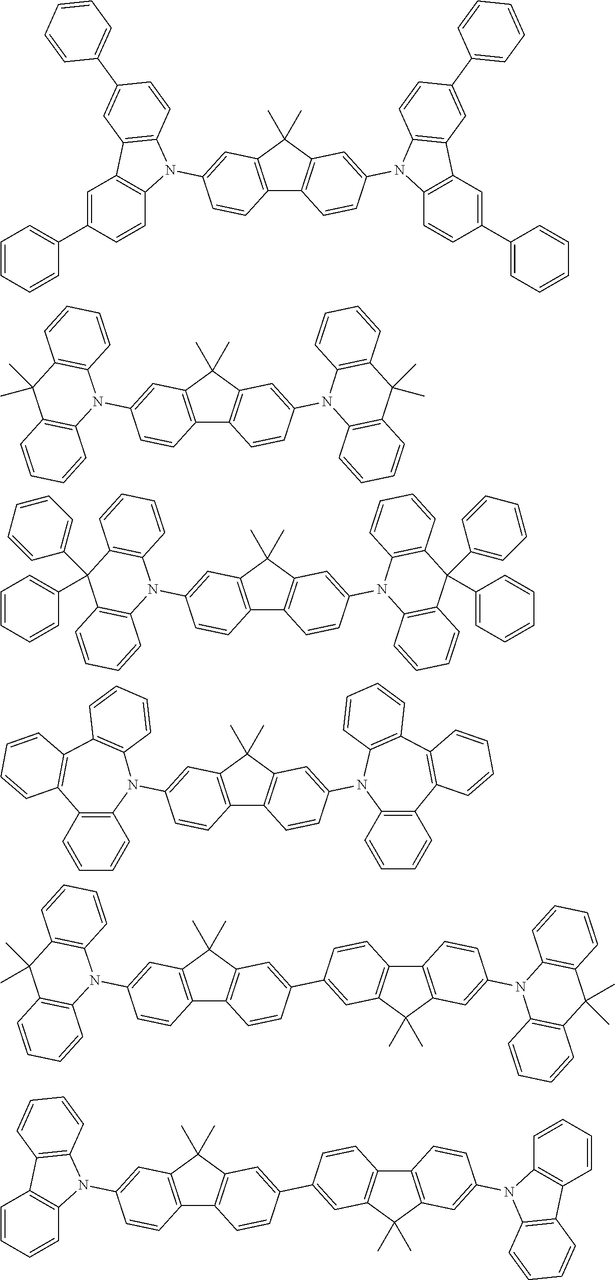

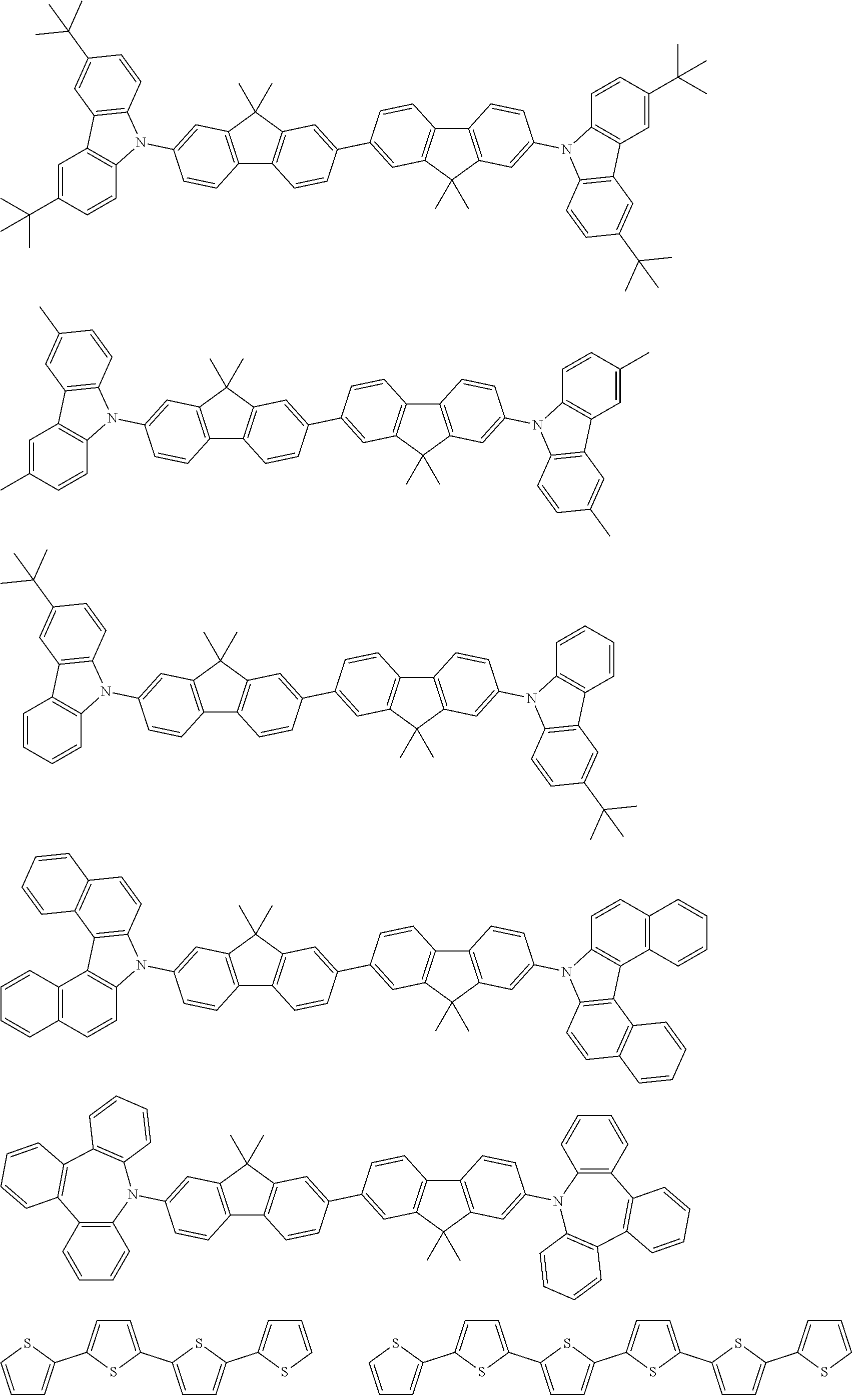

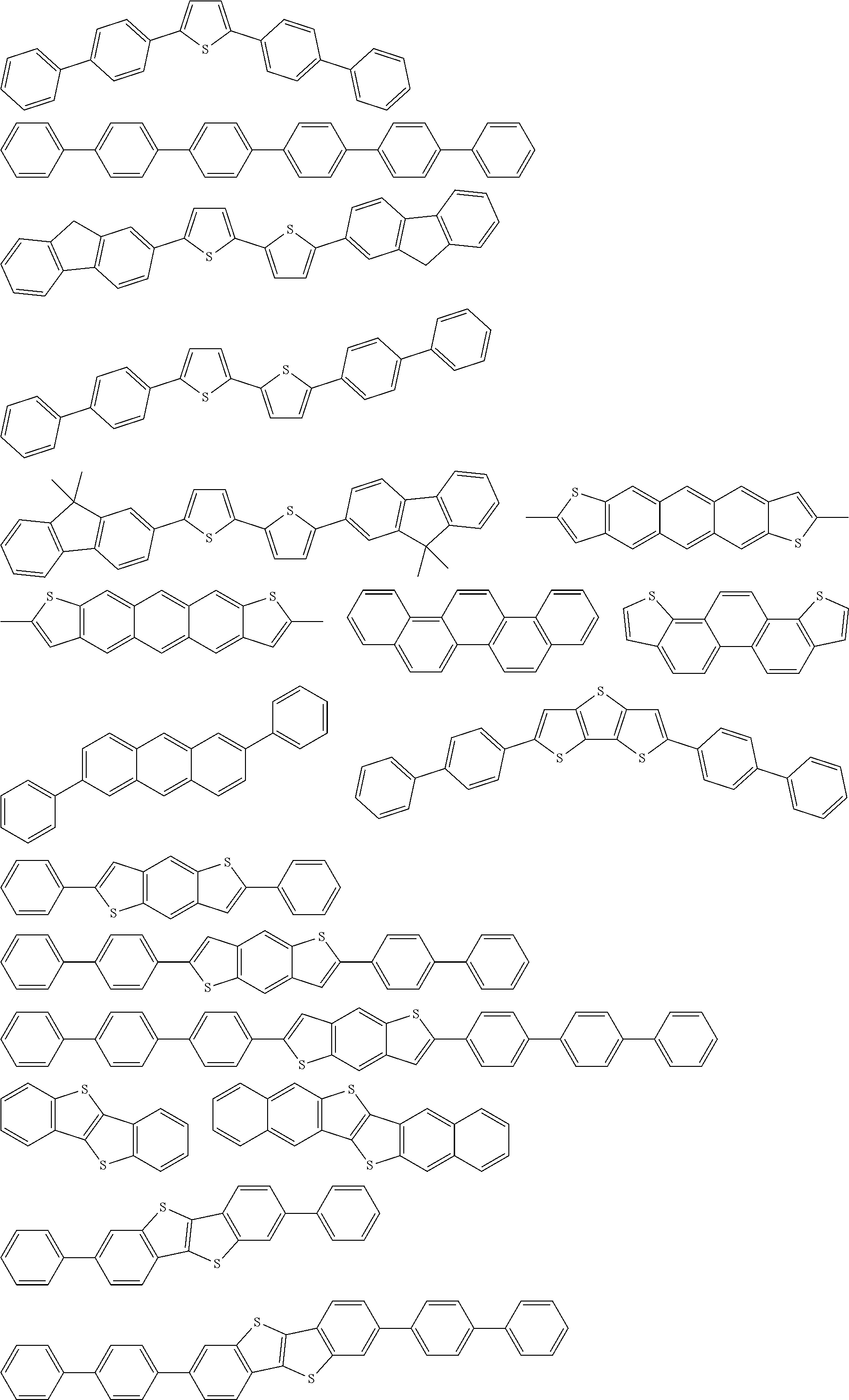

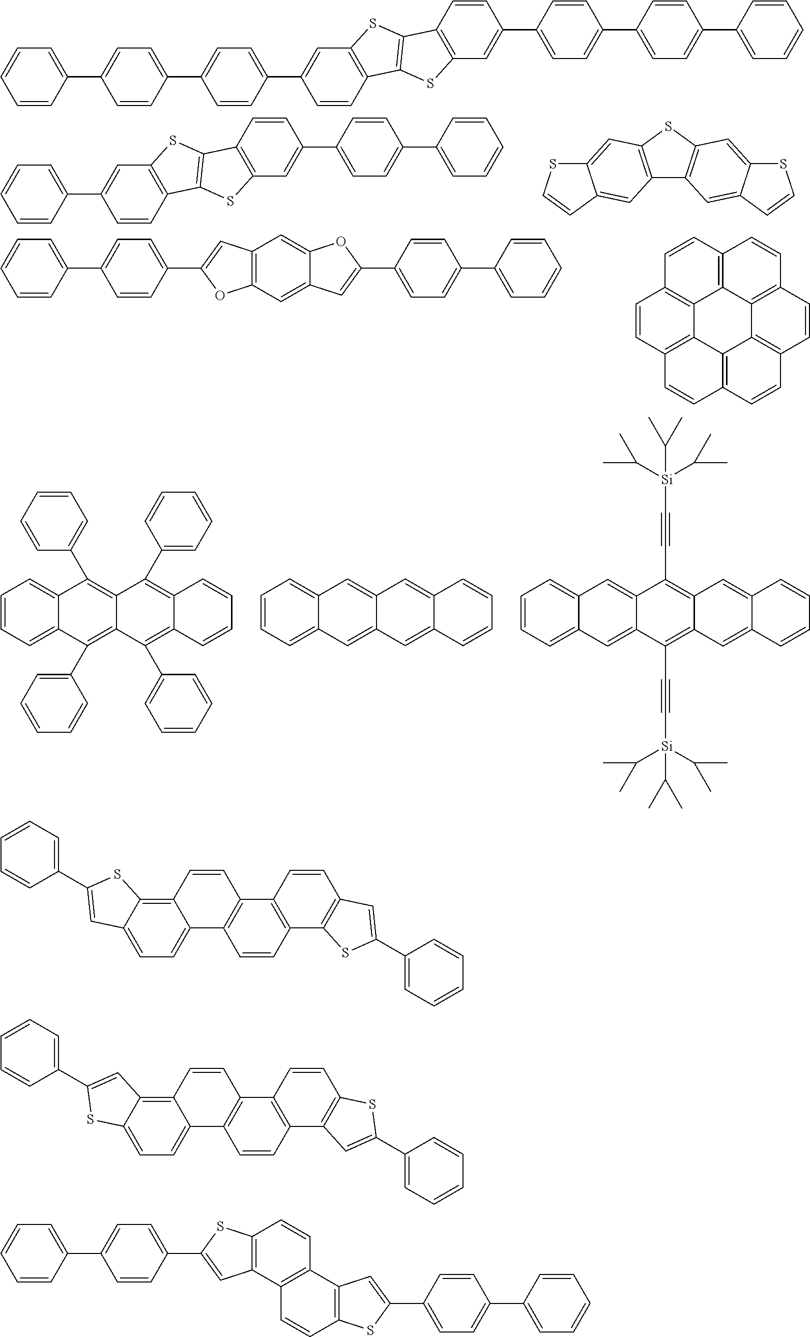

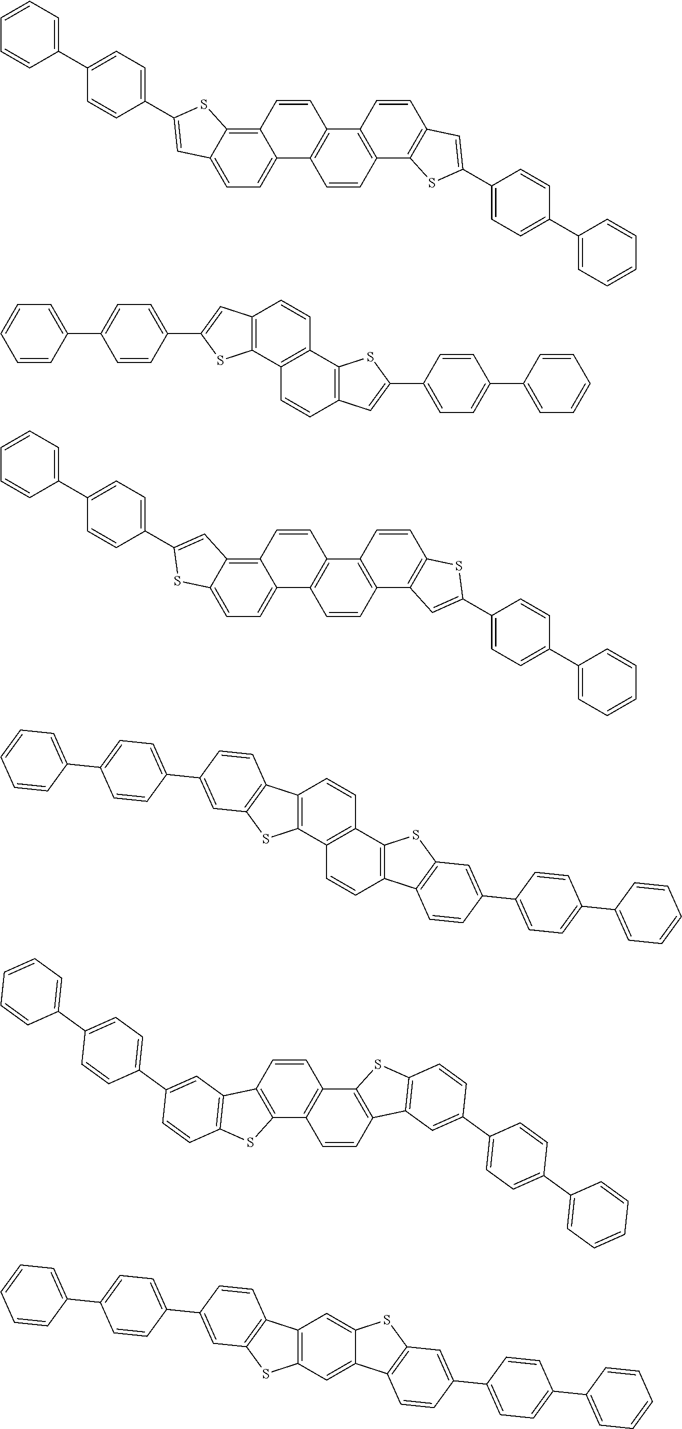 [SMILES: c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4ccc5c(ccc6cc(-c7ccc(-c8ccccc8)cc7)sc65)c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1 ; c1ccc(-c2ccc(-c3ccc4c(c3)sc3c4ccc4c3ccc3c5ccc(-c6ccc(-c7ccccc7)cc6)cc5sc34)cc2)cc1 ; c1ccc(-c2ccc(-c3ccc4sc5c(ccc6c5ccc5c7cc(-c8ccc(-c9ccccc9)cc8)ccc7sc56)c4c3)cc2)cc1 ; c1ccc(-c2ccc(-c3ccc4c(c3)sc3cc5c(cc34)sc3cc(-c4ccc(-c6ccccc6)cc4)ccc35)cc2)cc1]

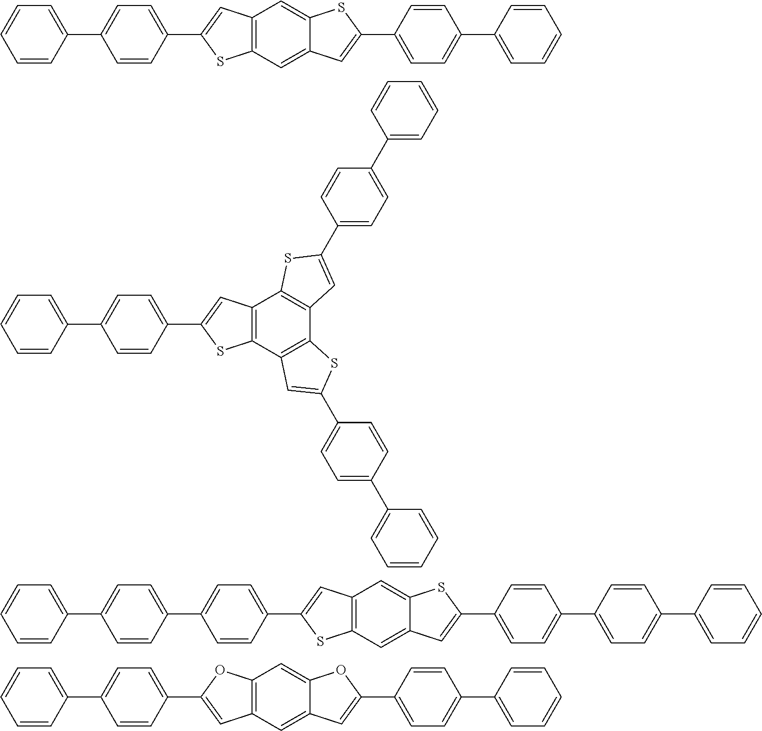

The difference in the ionization potential between the specific compound and the p-type organic semiconductor is preferably 0.1 eV or more.

The p-type organic semiconductor may be used alone, or two or more types thereof may be used in combination.

In a case where the photoelectric conversion film contains the p-type organic semiconductor, a content of the p-type organic semiconductor in the photoelectric conversion film (film thickness of p-type organic semiconductor in terms of single layer/film thickness of photoelectric conversion film×100) is preferably 15% to 75% by volume, more preferably 20% to 60% by volume, and still more preferably 25% to 50% by volume.

The photoelectric conversion film containing the specific compound is a non-light emitting film, and has a feature different from organic light emitting diodes (OLEDs). The non-light emitting film means a film having a light emission quantum efficiency of 1% or less, and the light emission quantum efficiency is preferably 0.5% or less, and more preferably 0.1% or less. The lower limit thereof is often 0% or more.

### <Coloring Agent>

The photoelectric conversion film preferably contains a coloring agent in addition to the above-described specific compound.

The coloring agent is a compound different from the specific compound.

As the coloring agent, an organic coloring agent is preferable.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, an imidazoquinoxaline coloring agent described in WO2020/013246A, WO2022/168856A, JP2023-10305A, and JP2023-10299A, and acceptor-donor-acceptor type coloring agent in which two acidic nuclei are bonded to a donor, donor-acceptor-donor type coloring agent in which two donors are bonded to an acceptor, and the like.

Among these, the organic coloring agent is preferably a cyanine coloring agent, an imidazoquinoxaline coloring agent, or an acceptor-donor-acceptor type coloring agent, and more preferably an imidazoquinoxaline coloring agent or an acceptor-donor-acceptor type coloring agent.

The maximal absorption wavelength of the coloring agent is preferably in the visible light region, more preferably in a wavelength range of 400 to 650 nm, and still more preferably in a wavelength range of 450 to 650 nm.

The coloring agent may be used alone, or two or more types thereof may be used in combination.

A content of the coloring agent with respect to the total content of the specific compound and the coloring agent in the photoelectric conversion film (=(film thickness of coloring agent in terms of single layer/(film thickness of specific compound in terms of single layer+film thickness of coloring agent in terms of single layer)×100)) is preferably 15% to 75% by volume, more preferably 20% to 60% by volume, and still more preferably 20% to 50% by volume.

### <Film Formation Method>

Examples of a film formation method of the above-described photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The film thickness of the photoelectric conversion film is preferably 10 to 1,000 nm, more preferably 50 to 800 nm, and still more preferably 50 to 500 nm.

### [Electrode]

The photoelectric conversion element preferably has an electrode.

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) and fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes, graphene, and the like. From the viewpoint of high conductivity and transparency, conductive metal oxides are preferable.

Generally, in a case where the conductive film is made thinner than a certain range, the resistance value rapidly increases in many cases. In the solid-state imaging element in which the photoelectric conversion element according to the present embodiment is incorporated, the sheet resistance may be 100 to 10,000Ω/□, and the degree of freedom of the film thickness range that can be reduced is large.

In addition, as the film thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO and FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum; conductive compounds (for example, titanium nitride (TiN)) such as oxides or nitrides of these metals; mixtures or laminates of these metals and conductive metal oxides; organic conductive materials such as polyaniline, polythiophene, and polypyrrole; and carbon materials such as carbon nanotubes and graphene.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film and Positive Hole Blocking Film]

It is preferable that the photoelectric conversion element includes one or more interlayers between the conductive film and the transparent conductive film, in addition to the photoelectric conversion film.

An example of the interlayer includes a charge blocking film. In a case where the photoelectric conversion element has this film, the characteristics (such as quantum efficiency and response speed) of the photoelectric conversion element to be obtained are more excellent. Examples of the charge blocking film include an electron blocking film and a positive hole blocking film.

### [Electron Blocking Film]

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the quantum efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### [Positive Hole Blocking Film]

A positive hole blocking film is an acceptor-property organic semiconductor material (a compound), and the n-type organic semiconductor described above can be used.

In addition, the positive hole blocking film may be formed with a plurality of films.

Examples of a method of producing a charge blocking film include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each film thickness of the charge blocking films (the electron blocking film and the positive hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### [Sealing Layer]

The photoelectric conversion element may further include a sealing layer.

The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, metal nitride, or metal nitride oxide which are dense and into which water molecules do not permeate.

Examples of the sealing layer include layers described in paragraphs [0210] to [0215] of JP2011-082508A, the contents of which are incorporated herein by reference.

### [Imaging Element]

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

### [Optical Sensor]

Examples of another application of the photoelectric conversion element include the photoelectric cell and the optical sensor, but the photoelectric conversion element of the embodiment of the invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention further includes the invention of compounds. The compound according to the embodiment of the present invention is the above-described specific compound, or a compound represented by Formula (2) (hereinafter, also referred to as an “intermediate A”), a compound represented by Formula (3) (hereinafter, also referred to as an “intermediate B”), or a compound represented by Formula (3c), which is an intermediate in a synthesis step of the specific compound.

### <Specific Compound>

Specific aspects and suitable aspects of the specific compound are as described above.

### <Intermediate A>

The intermediate A (compound represented by Formula (2)) is represented by the following structural formula.

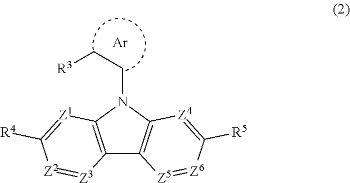

In Formula (2), Z¹ to Z⁶ each independently represent —CR^{X1}═ or a nitrogen atom. R^{X1} represents a hydrogen atom or a substituent.

In a case where adjacent two of Z¹ to Z⁶ are —CR^{X1}═, two R^{X1}'s may be bonded to each other to form a ring.

Specific aspects and suitable aspects of Z¹ to Z⁶ in Formula (2) are the same as the specific aspects and suitable aspects of Z¹ to Z⁶ in Formula (1).

In Formula (2), R³ represents a substituent selected from the substituent group T.

The substituent group T: a linear aliphatic hydrocarbon group, a branched aliphatic hydrocarbon group, a cyclic aliphatic hydrocarbon group, and an aromatic ring group which may have a substituent selected from a substituent group R^{Ar3} and does not contain a nitrogen atom.

The number of carbon atoms of the linear aliphatic hydrocarbon group is preferably 1 to 10, more preferably 1 to 6, and still more preferably 1 to 3.

The number of carbon atoms of the branched aliphatic hydrocarbon group is preferably 3 to 10, more preferably 3 to 6, and still more preferably 3 to 5.

The number of carbon atoms of the cyclic aliphatic hydrocarbon group is preferably 3 to 10 and more preferably 3 to 8.

In addition, in the substituent group T, the linear aliphatic hydrocarbon group, the branched aliphatic hydrocarbon group, and the cyclic aliphatic hydrocarbon group may have an ethereal oxygen atom.

In the substituent group T, the substituent selected from the substituent group R^{Ar3} is as follows.

The substituent group R^{Ar3}: a linear aliphatic hydrocarbon group, a branched aliphatic hydrocarbon group, a cyclic aliphatic hydrocarbon group, a halogen atom, and aromatic ring group which does not contain a nitrogen atom.

Specific aspects and suitable aspects of each group exemplified in the substituent group R^{Ar3} are the same as the specific aspects and suitable aspects of each group exemplified in the substituent group T.

In the substituent group R^{Ar3}, the linear aliphatic hydrocarbon group, the branched aliphatic hydrocarbon group, and the cyclic aliphatic hydrocarbon group may have an ethereal oxygen atom and may be substituted with a halogen atom.

Among the substituents selected from the substituent group T, a linear alkyl group having 1 to 3 carbon atoms, a methoxy group, a branched alkyl group having 3 to 5 carbon atoms, or a cyclic alkyl group having 3 carbon atoms (cyclopropyl group) is preferable.

In Formula (2), R⁴ and R⁵ each independently represent an iodine atom, *—O—S(═O)₂R^{f}, a bromine atom, a chlorine atom, a fluorine atom, a formyl group, *—Sn(R^{Sn})₃, *—B(R^{B1})₂, or *—B⁻(R^{B2})₃M⁺.

R¹ represents a perfluoroalkyl group having 1 to 6 carbon atoms. R^{Sn}, R^{B1}, and R^{B2} each independently represent a substituent, and a plurality of R^{Sn}'s, R^{B1}'s, and R^{B2}'s may be the same as or different from each other. R^{B1}'s and R^{B2}'s may be bonded to each other to form a ring structure. M⁺ represents a monovalent metal cation.

As the perfluoroalkyl group represented by R^{f}, a trifluoromethyl group is preferable.

Examples of the substituent represented by R^{Sn} include an aliphatic hydrocarbon group which may have a substituent, an aromatic ring group which may have a substituent, and an aliphatic heterocyclic group which may have a substituent. Examples of the substituent which may be included in each of the above-described groups include the group exemplified by the above-described substituent W.

Among these, the substituent represented by R^{Sn} is preferably an aliphatic hydrocarbon group which may have an aromatic ring group, more preferably an alkyl group having 1 to 10 carbon atoms, and still more preferably a methyl group or a butyl group.

The substituent represented by R^{B1} is not particularly limited as long as it is a substituent in an organic boron compound generally used in an aromatic coupling reaction, and examples thereof include a hydroxy group and an alkoxy group.

In a case where R^{B1}'s are bonded to each other to form a ring, the formed ring may be an aromatic ring (for example, a benzene ring) or a non-aromatic ring.

Examples of the group represented by R^{B1} include a group represented by Formula (B1) and a group represented by Formula (B2).

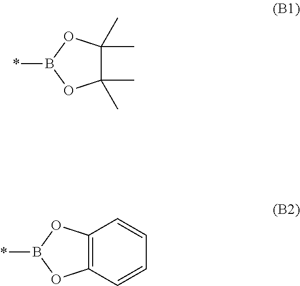

The substituent represented by R^{B2} is not particularly limited as long as it is a substituent in an organic boron compound generally used in an aromatic coupling reaction, and examples thereof include a fluorine atom and an alkoxy group.

Examples of the monovalent metal cation represented by M⁺ include monovalent metal cations of a lithium ion, a potassium ion, a sodium ion, a rubidium ion, and a cesium ion.

In a case where R^{B2}'s are bonded to each other to form a ring, the formed ring may be an aromatic ring (for example, a benzene ring) or a non-aromatic ring.

Among the three R^{B2}'s, two R^{B2}'s may be bonded to each other to form a ring, or the three R^{B2}'s may be bonded to each other to form a ring.

Examples of the group represented by R^{B2} include a group represented by Formula (B3). In Formula (B3), M⁺ represents the monovalent metal cation.

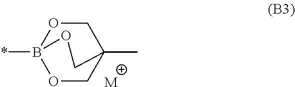

From the viewpoints of the manufacturing suitability of the specific compound and the ease of functional group conversion with R⁴ and R⁵ as a starting point, as R⁴ and R⁵, *—O—S(═O)₂R^{f}, a bromine atom, a chlorine atom, or a fluorine atom is preferable, and a bromine atom or a formyl group is more preferable.

In Formula (2), Ar represents an aromatic ring which contains two or more carbon atoms as a ring member atom and does not contain a nitrogen atom as a ring member atom.

The aromatic ring represented by Ar may be any of a monocyclic ring or a polycyclic ring, and may be any of an aromatic hydrocarbon ring or an aromatic heterocyclic ring (an aromatic heterocyclic ring which does not contain a nitrogen atom as a ring member atom). Specific aspects of the monocyclic aromatic ring, the polycyclic aromatic ring, the aromatic hydrocarbon ring, and the aromatic heterocyclic ring are as described above.

The number of ring member atoms in the aromatic ring represented by Ar is preferably 4 to 15, more preferably 4 to 10, and still more preferably 4 to 6.

Among these, as the aromatic hydrocarbon ring represented by Ar, a benzene ring or a naphthalene ring is preferable.

In addition, as the aromatic heterocyclic ring represented by Ar, a thiophene ring, a benzofuran ring (for example, a 2,3-benzofuran ring or the like), or a benzothiophene ring (for example, a benzo[b]thiophene ring or the like) is preferable.

The aromatic ring represented by Ar may be substituted with a substituent selected from the substituent group T or a halogen atom. Specific aspects and suitable aspects of the substituent selected from the substituent group T are as described above.

In a case where the aromatic ring represented by Ar is substituted with a substituent selected from the substituent group T or a halogen atom, the number of substitutions is not particularly limited, but is preferably 1 to 4 and more preferably 1 or 2.

In a case where the aromatic ring represented by Ar has the substituent selected from the substituent group T, the substituent selected from the substituent group T, which is represented by R³, and the substituent selected from the substituent group T, which is contained in the aromatic ring represented by Ar, may be bonded to each other to form a non-aromatic ring.

In addition, in a case where the aromatic ring represented by Ar is substituted with a plurality of substituents selected from the substituent group T, the plurality of substituents may be bonded to each other to form a non-aromatic ring.

Examples of the non-aromatic ring include an aliphatic ring, and examples thereof include an aliphatic ring having 4 to 6 carbon atoms.

### <Manufacturing Method of Intermediate A>

Examples of the manufacturing method of the intermediate A include a method of introducing a group represented by R⁴ and a group represented by R⁵ into a compound (N-aryl-substituted carbazole) in which an aryl group is present on a nitrogen atom of carbazole by a reaction such as halogenation or lithiation (lithiation).

However, as described in “Helvetica Chimica Acta (2006), 89(6), 1123-1139” or the like, in a case where the N-aryl-substituted carbazole is halogenated or lithiated, it is considered that it is generally difficult to obtain the intermediate A which is a compound in which the 2-position and the 7-position are substituted, since the reaction proceeds at the 3-position and the 6-position.

### Step P1

In view of the above-described contents, the present inventors have conducted various studies, and as a result, they have found that the intermediate A (the compound represented by Formula (2)) can be efficiently manufactured by a manufacturing method of a compound, which includes a step of reacting a compound represented by Formula (2a) with a compound represented by Formula (X) to manufacture a compound represented by Formula (2b) (hereinafter, also referred to as Step P1).

The compound represented by Formula (2b) is an aspect in which R⁴ and R⁵ in Formula (2) are each independently an iodine atom, *—O—S(═O)₂R^{f} a bromine atom, a chlorine atom, or a fluorine atom, and as described in detail later, among compounds represented by Formula (2), a compound in which R⁴ and R⁵ are each independently a formyl group, *—Sn(R^{Sn})₃, *—B(R^{B1})₂, or *—B⁻(R^{B2})₃M⁺ can be obtained by converting a group represented by R^{L4} and a group represented by R^{L5} in the compound represented by Formula (2b) into a formyl group, *—Sn(R^{Sn})₃, *—B(R^{B1})₂, or *—B⁻(R^{B2})₃M⁺.

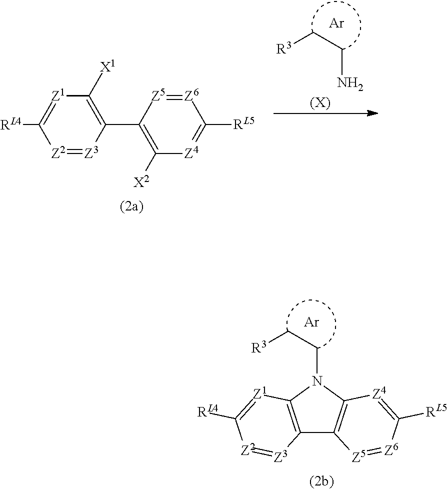

In Formula (2a), Z¹ to Z⁶ each independently represent —CR^{X1}═ or a nitrogen atom. R^{X1} represents a hydrogen atom or a substituent. In a case where adjacent two of Z¹ to Z⁶ are —CR^{X1}═, two R^{X1}'s may be bonded to each other to form a ring.

Z¹ to Z⁶ have the same meanings as Z¹ to Z⁶ in Formula (2), and suitable aspects thereof are also the same.

In Formula (2a), R^{L4} and R^{L5} each independently represent *—O—S(═O)₂R^{f}, a bromine atom, a chlorine atom, or a fluorine atom.

X¹ and X² each independently represent an iodine atom, *—O—S(═O)₂R^{f}, a bromine atom, or a chlorine atom. R^{f} has the same meaning as R^{f} in Formula (2).

Provided that R^{L4}, R^{L5}, X¹, and X² satisfy the following requirement.

The requirement: in a case where an iodine atom is assigned to a first rank, *—O—S(═O)₂R^{f} is assigned to a second rank, a bromine atom is assigned to a third rank, a chlorine atom is assigned to a fourth rank, and a fluorine atom is assigned to a fifth rank, and in a case where the ranks are higher from the first rank to the fifth rank, both a rank of a group represented by R^{L4} and a rank of a group represented by R^{L5} are higher than a rank of a group represented by X¹ and are higher than a rank of a group represented by X².

In the requirement, the order from the first position to the fifth position represents the difficulty of leaving as a leaving group, which means that the fifth position is difficult to be eliminated. That is, the higher the rank is (closer to the fifth position), the more difficult the elimination is.

Therefore, in a case where R^{L4}, R^{L5}, X¹, and X² satisfy the requirements, both the group represented by X¹ and the group represented by X² are more easily eliminated than the group represented by R^{L4} and more easily eliminated than the group represented by R^{L5}. Therefore, the intramolecular cyclization reaction proceeds preferentially over the intermolecular reaction.

For example, in a case where X¹ is an iodine atom at the first position, X² is an iodine atom at the first position, R^{L4} is a bromine atom at the third position, and R^{L5} is a bromine atom at the third position, the ranks of R^{L4} and R^{L5} (both at the third position) are higher than the ranks of X¹ and X² (both at the first position), and thus the above requirement is satisfied.

In a case where X¹ is a bromine atom at the third position, X² is a bromine atom at the third position, R^{L4} is an iodine atom at the first position, and R^{L5} is an iodine atom at the first position, the ranks of R^{L4} and R^{L5} (both at the first position) are lower than the ranks of X¹ and X² (both at the third position), and thus the above requirement is not satisfied.

Examples of the combination of R^{L4}, R^{L5}, X¹, and X² satisfying the above-described requirements include the following Examples 1 to 4.
Example 1: X¹=I, X²=I, R^{L4}=Br, and R^{L5}=Br
Example 2: X¹=I, X²=*—O—S(═O)₂R^{f}, R^{L4}=Br, and R^{L5}=Br
Example 3: X¹=I, X²=I, R^{L4}=Br, and R^{L5}=Cl
Example 4: X¹=Br, X²=Br, R^{L4}=Cl, and R^{L5}=Cl

Among the combinations satisfying the requirements, the combination of Example 1 is preferable.

In Formula (X), R³ represents a substituent selected from the substituent group T.

Ar represents an aromatic ring which contains two or more carbon atoms as a ring member atom and does not contain a nitrogen atom as a ring member atom. The aromatic ring represented by Ar may be substituted with a substituent selected from the substituent group T or a halogen atom.

In a case where the aromatic ring represented by Ar has the substituent selected from the substituent group T, the substituent selected from the substituent group T, which is represented by R³, and the substituent selected from the substituent group T, which is contained in the aromatic ring represented by Ar, may be bonded to each other to form a non-aromatic ring.

In a case where the aromatic ring represented by Ar is substituted with a plurality of substituents selected from the substituent group T, the plurality of substituents may be bonded to each other to form a non-aromatic ring.

R³ and Ar in Formula (X) have the same meanings as R³ and Ar in Formula (2), and the same applies to the suitable aspects thereof.

In Formula (2b), Z¹ to Z⁶, R^{L4}, and R^{L5} have the same meanings as Z¹ to Z⁶, R^{L4}, and R^{L5} in Formula (2a).

R³ and Ar have the same meanings as R³ and Ar in Formula (X).

Step P1 described above is typically performed under Buchwald-Hartwig cross-coupling conditions in many cases. More specifically, Step P1 is preferably carried out in the presence of an organometallic catalyst and a base.

Examples of the organometallic catalyst include a palladium catalyst, and more specific examples thereof include palladium salts such as palladium chloride, palladium acetate, palladium trifluoroacetate, and palladium nitrate; complex compounds such as π-allylpalladium chloride dimer, palladium acetylacetonate, dipalladium tris(dibenzylideneacetone), palladium bis(dibenzylideneacetone), dichlorobis(acetonitrile)palladium, and dichlorobis(benzonitrile)palladium; and palladium complexes having a tertiary phosphine as a ligand, such as dichlorobis(triphenylphosphine)palladium, tetrakis(triphenylphosphine)palladium, dichloro(1,1′-bis(diphenylphosphino)ferrocene)palladium, bis(tri-tert-butylphosphine)palladium, bis(tricyclohexylphosphine)palladium, and dichlorobis(tricyclohexylphosphine)palladium.

The palladium catalyst may be prepared in a reaction system by adding a tertiary phosphine to a palladium salt or a complex compound.

Among these, as the organometallic catalyst, a palladium complex having a tertiary phosphine as a ligand is preferable, a palladium complex having a tertiary phosphine having at least one aryl group as a ligand is more preferable, and a palladium complex having a triaryl phosphine as a ligand is still more preferable.

Examples of the aryl group which may be contained in the tertiary phosphine include a phenyl group which may have a group exemplified by the substituent W.

Examples of the base include a base containing an alkali metal and a tertiary amine, and the base containing an alkali metal is preferable.

As the base containing an alkali metal, an alkali metal alkoxide (for example, sodium methoxide, sodium ethoxide, potassium t-butoxide, and the like), or an alkali metal carbonate, phosphate, hydroxide, and fluoride is preferable, an alkali metal alkoxide is more preferable, and an alkoxide consisting of a tert-butoxide anion and an alkali metal is still more preferable.

Examples of the alkali metal include lithium, potassium, sodium, and cesium, and lithium, potassium, or sodium is preferable.

Examples of the reaction solvent in Step P1 include toluene, tetrahydrofuran, 1,4-dioxane, 1,2-dichlorobenzene, benzene, xylene, mesitylene, anisole, chlorobenzene, dimethoxyethane, dimethylformamide (DMF), cyclopentyl methyl ether, 4-methyltetrahydropyran, acetonitrile, alcohol, and ionic liquids, and toluene is preferable.

The reaction temperature is a temperature at which the reaction solvent to be used is heated and refluxed in many cases, and is preferably 50° C. to 200° C. and more preferably 90° C. to 150° C.

### Step P2

In the compound represented by Formula (2b), by further performing a step of converting the group represented by R^{L4} and the group represented by R^{L5} into a formyl group, *—Sn(R^{Sn})₃, *—B(R^{B1})₂, or *—B⁻(R^{B2})₃M⁺ (hereinafter, also referred to as “Step P2”), a compound in which R⁴ and R⁵ in the compound represented by Formula (2) are a formyl group, *—Sn(R^{Sn})₃, *—B(R^{B1})₂, or *—B⁻(R^{B2})₃M⁺ is obtained.

R^{Sn}, R^{B1}, and R^{B2} have the same meanings as R^{Sn}, R^{B1}, and R^{B2} in the intermediate A (the compound represented by Formula (2)), and suitable aspects thereof are also the same.

### Step P2A

Examples of the step of converting the group represented by R^{L4} and the group represented by R^{L5} in the compound represented by Formula (2b) into a formyl group include a step of reacting the compound represented by Formula (2b) with a formylating agent.

As the formylating agent, known agents can be used, and examples thereof include N,N-disubstituted formamide, an orthoformic acid ester, and a compound represented by Formula (B″).

Examples of the N,N-disubstituted formamide include a compound represented by Formula (B).

OHC—NR^{Y2}₂ Formula (B)

In Formula (B), R¹² represents an organic group. A plurality of R^{Y2}'s may be the same as or different from each other.

Examples of the organic group include an aliphatic hydrocarbon group which may have a substituent, an aromatic ring group which may have a substituent, and an aliphatic heterocyclic group which may have a substituent, and among these, an aliphatic hydrocarbon group or an aromatic ring group is preferable.

Examples of the compound represented by Formula (B) include N,N-dimethylformamide (DMF), N-(diethylcarbamoyl)-N-methoxyformamide, 1-formylpiperidine, 4-formylmorpholine, N-methylformanilide, and N-formylsaccharin, and among these, DMF is preferable.

Examples of the orthoformic acid ester include a compound represented by Formula (B′).

HC(OR^{Y3})₃ Formula (B′)

In Formula (B′), R^{Y3} represents an alkyl group. A plurality of R^{Y3}'s may be the same as or different from each other. The alkyl group is preferably an alkyl group having 1 to 6 carbon atoms and more preferably a methyl group or an ethyl group.

The compound represented by Formula (B″) is as follows.

In Formula (B″), R represents an alkyl group having 1 to 6 carbon atoms.

Among these, N-methoxyethylaniline is preferable as the compound represented by Formula (B″).

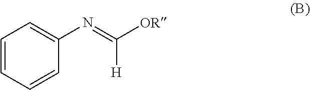

In Step P2A, typically, the group represented by R⁴ and the group represented by R⁵ in the compound represented by Formula (2b) are converted into a metal active species using a metalating reagent, and then reacted with the above-described formylating agent in many cases. The metalating reagent to be used and reaction conditions are not particularly limited, and known metalating reagents and reaction conditions can be applied.

Among these, as the metalating reagent used in Step P2A, a lithium reagent or a magnesium reagent is preferable.

Among these, as the lithium reagent, an organolithium reagent is preferable, and examples thereof include alkyl lithium such as n-butyl lithium, sec-butyl lithium, and tert-butyl lithium.

Examples of the magnesium reagent include an organic magnesium reagent (Grignard reagent and the like). It is noted that examples of the magnesium reagent also include magnesium metal.

### Step P2B

Examples of the step of converting the group represented by R^{L4} and the group represented by R^{L5} in the compound represented by Formula (2b) to *—Sn(R^{Sn})₃ include a step of reacting the compound represented by Formula (2b) with a compound represented by Formula (Y) (hereinafter, also referred to as “Step P2B”).

(R^{Sn})₃Sn—X^{a} Formula (Y)

In Formula (Y), R^{Sn} has the same meaning as R^{Sn} in *—Sn(R^{Sn})₃ exemplified as R⁴ and R⁵ in Formula (2).

Examples of the substituent represented by R^{Sn} include an aliphatic hydrocarbon group which may have a substituent, an aromatic ring group which may have a substituent, and an aliphatic heterocyclic group which may have a substituent. Examples of the substituent which may be included in each of the above-described groups include the group exemplified by the above-described substituent W.

Among these, the substituent represented by R^{Sn} is preferably an aliphatic hydrocarbon group which may have an aromatic ring group, more preferably an alkyl group having 1 to 10 carbon atoms, and still more preferably a methyl group or a butyl group.

In Formula (Y), X^{a} represents an iodine atom, *—O—S(═O)₂R^{f} a bromine atom, a chlorine atom, or *—Sn(R^{Sn})₃. R^{f} has the same meaning as R^{f} in Formula (2). Among these, X^{a} is preferably an iodine atom, *—O—S(═O)₂R^{f}, a bromine atom, or a chlorine atom, and more preferably a chlorine atom.

In Step P2B, typically, the group represented by R^{L4} and the group represented by R^{L5} in the compound represented by Formula (2b) are converted into lithium using an organolithium reagent, and then the compound represented by Formula (Y) is reacted. The reaction conditions are not particularly limited as long as the conditions are generally for lithiation.

Examples of the organolithium reagent include alkyl lithium such as n-butyl lithium, sec-butyl lithium, and tert-butyl lithium.

In addition, Step P2B can also be performed in the presence of a palladium catalyst. As specific reaction conditions, a synthesis method described in “J. Org. Chem. 2016, 81, 8, 3356-3363” can be referred to.

As the palladium catalyst, those exemplified as the palladium catalyst in Step P1 can be used.

### Step P2C

Examples of the step of converting the group represented by R^{L4} and the group represented by R^{L5} in the compound represented by Formula (2b) into *—B(R^{B1})₂ or *—B⁻(R^{B2})₃M⁺ include a step of reacting the compound represented by Formula (2b) with a borylating agent.

As the borylating agent, known agents can be used, and examples thereof include a compound represented by Formula (Z).

X^{b}—B(OR^{B3})₂ Formula (Z)

In Formula (Z), X^{b} represents an iodine atom, *—O—S(═O)₂R^{f} a bromine atom, a chlorine atom, *—B(OR^{B3})₂, *—OR^{B4}, or *—Si(R^{B5})₃.

R^{f} has the same meaning as R^{f} in Formula (2).

R^{B3}, R^{B4}, and R^{B5} each independently represent an aliphatic hydrocarbon group which may have a substituent, an aromatic ring group which may have a substituent, or an aliphatic heterocyclic group which may have a substituent. A plurality of R^{B3}'s and a plurality of R^{B5}'s may be the same as or different from each other.

Among these, R^{B3} and R^{B4} are each preferably an aliphatic hydrocarbon group, more preferably an alkyl group, and still more preferably an alkyl group having 1 to 6 carbon atoms.

R^{B3}'s may be bonded to each other to form a ring, and the compound represented by Formula (Z) is preferably a compound represented by Formula (Z′).

In a case where R^{B3}'s are bonded to each other to form a ring, the group represented by *—B(OR^{B3})₂ is preferably a group represented by Formula (B1) or Formula (B2).

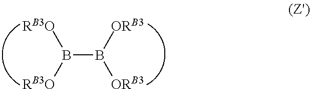

In *—Si(R^{B5})₃, among these, R^{B5} is preferably an aliphatic hydrocarbon group or an aromatic ring group, and more preferably an alkyl group having 1 to 6 carbon atoms or a phenyl group.

In Step P2C, typically, the group represented by R^{L4} and the group represented by R^{L5} in the compound represented by Formula (2b) are converted into lithium using an organolithium reagent, and then the compound represented by Formula (Z) is reacted. The reaction conditions are not particularly limited as long as the conditions are generally for lithiation, and specific examples of the organolithium reagent are as described above.

In addition, it is also effective to convert the group represented by R⁴ and the group represented by R⁵ into lithium, and then react a compound represented by Formula (Z) to convert the group represented by R⁴ and the group represented by R⁵ into *—B(R^{B1})₂ or *—B⁻(R^{B2})₃M⁺. As specific reaction conditions, the synthesis method described in “Org. Lett. 2006, 8, 18, 4071-4074” can be referred to.

In addition, Step P2C is performed in the presence of a palladium catalyst in many cases. As specific reaction conditions, the synthesis method described in “European Polymer Journal (2019), 112, 283-290” can be referred to.

As the palladium catalyst and the base that can be used in the present synthesis method, those exemplified as the palladium catalyst and the base in Step P1 can be used.

In addition to the above-described reaction conditions, Step P2C can also be performed without using a transition metal catalyst. Examples of such reaction conditions include a case where the compound represented by Formula (2b) are reacted with the compound represented by Formula (Z) in which X^{b} is *—Si(R^{B5})₃, and as specific reaction conditions, a synthesis method described in “J. Am. Chem. Soc. 2012, 134, 19997-20000” can be referred to.

Specific examples of the compound represented by Formula (2) will be shown below, but the present invention is not limited thereto.

R⁴ and R⁵ in the following exemplary compounds each independently represent a fluorine atom, a chlorine atom, a bromine atom, an iodine atom, *—Sn(n-Bu)₃, *—SnMe₃, *—B(OH)₂, *—BF₃M⁺ (M⁺ represents a monovalent metal cation), a formyl group, or a group represented by any of Formulae (B1) to (B3).

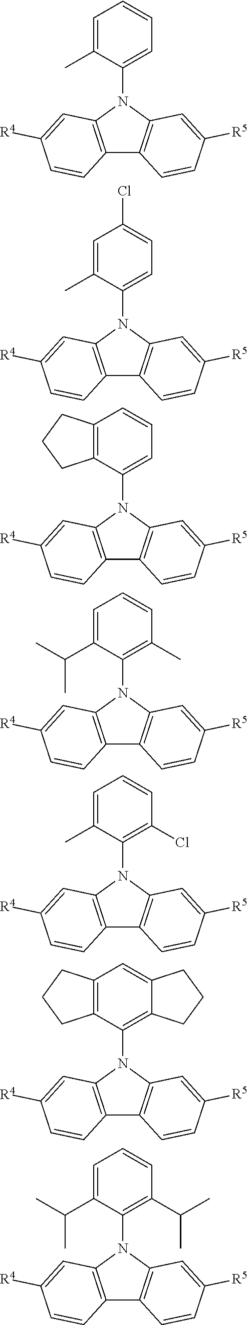

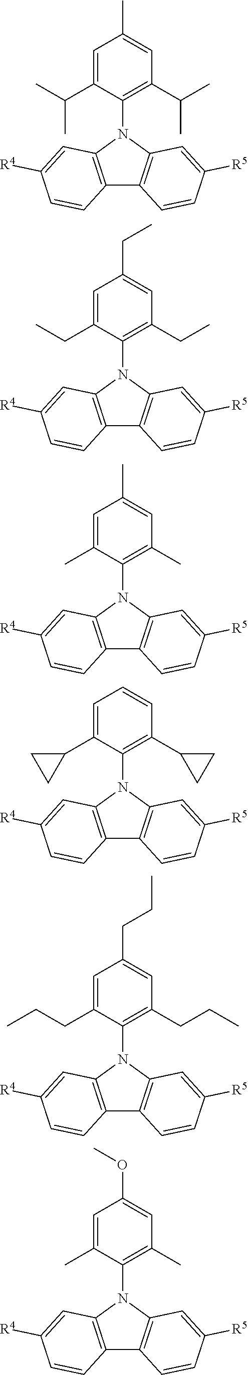

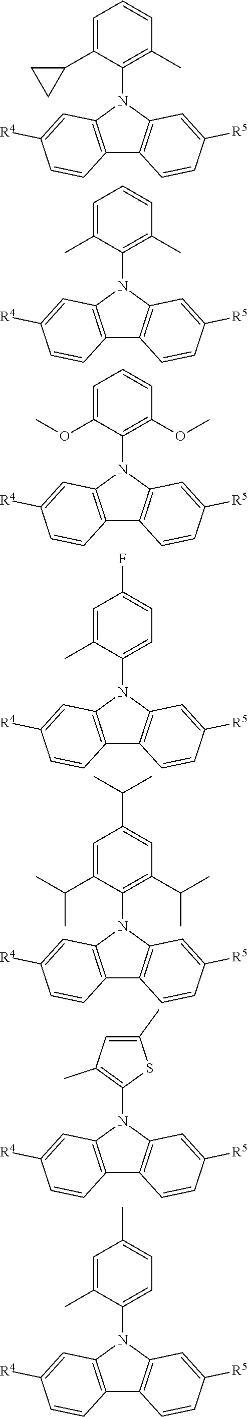

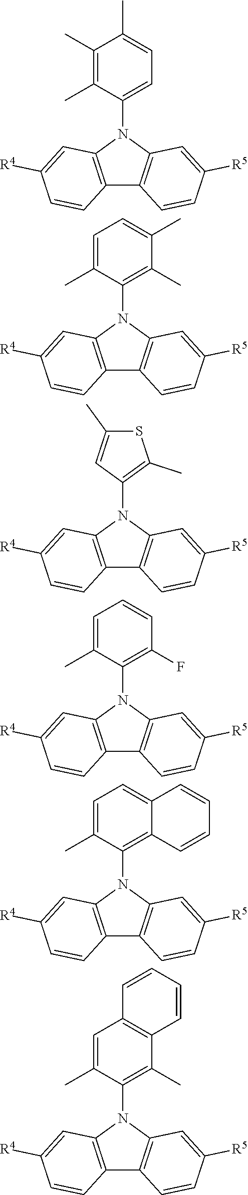

### <Intermediate B>

Next, the intermediate B (compound represented by Formula (3)) will be described in detail. The intermediate B is represented by the following structural formula.

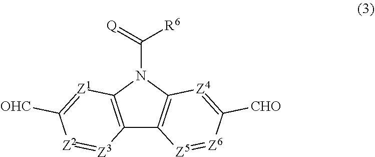

In Formula (3), Z¹ to Z⁶ each independently represent —CR^{X1}═ or a nitrogen atom. R^{X1} represents a hydrogen atom or a substituent.

In a case where adjacent two of Z¹ to Z⁶ are —CR^{X1}═, two R^{X1}'s may be bonded to each other to form a ring.

Specific aspects and suitable aspects of Z¹ to Z⁶ in Formula (3) are the same as the specific aspects and suitable aspects of Z¹ to Z⁶ in Formula (1).

In Formula (3), Q represents an oxygen atom or a sulfur atom, and an oxygen atom is preferable.

R⁶ represents a substituent selected from a substituent group U.

The substituent group U: an aliphatic hydrocarbon group which may have a substituent, an aromatic ring group which may have a substituent, and an aliphatic heterocyclic group which may have a substituent.

In the substituent group U, as the aliphatic hydrocarbon group which may have a substituent, a linear aliphatic hydrocarbon group having 1 to 3 carbon atoms, which may have a halogen atom, a branched aliphatic hydrocarbon group having 3 to 5 carbon atoms, which may have a halogen atom, or a cyclic aliphatic hydrocarbon group having 3 to 6 carbon atoms, which may have a halogen atom is preferable.

In the substituent group U, as the aromatic ring group which may have a substituent, the aromatic ring group which may have a substituent selected from the substituent group Rr is preferable, and an aromatic ring group having 4 to 10 ring member atoms, which may have a linear aliphatic hydrocarbon group having 1 to 3 carbon atoms, a branched aliphatic hydrocarbon group having 3 to 5 carbon atoms, or a cyclic aliphatic hydrocarbon group having 3 to 8 carbon atoms is preferable, and a phenyl group which may have a linear aliphatic hydrocarbon group having 1 to 3 carbon atoms, a branched aliphatic hydrocarbon group having 3 to 5 carbon atoms, or a cyclic aliphatic hydrocarbon group having 3 to 8 carbon atoms is more preferable.

### <Manufacturing Method of Intermediate B>

The manufacturing method of an intermediate B according to the embodiment of the present invention is a manufacturing method of a compound, including a step 1 of reacting a compound represented by Formula (3a) with a compound represented by Formula (A) to obtain a compound represented by Formula (3b), which has a protective group represented by SiR^{Y1}₃, a step 2 of reacting the compound represented by Formula (3b) with a metalating reagent, reacting the reacted compound with a formylating agent, and further deprotecting the protective group to obtain a compound represented by Formula (3c), and a step 3 of reacting the compound represented by Formula (3c) with a compound represented by Formula (C) to obtain a compound represented by Formula (3).

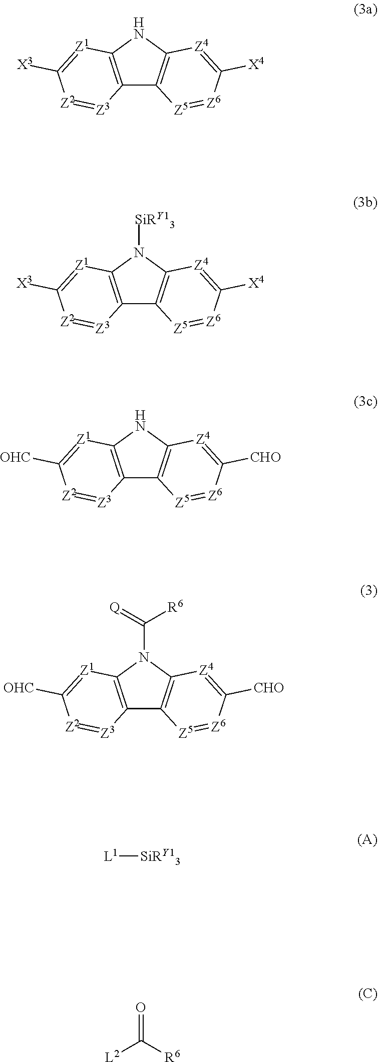

In Formulae (3a) to (3c), Z¹ to Z⁶ have the same meanings as Z¹ to Z⁶ in Formula (3), and suitable aspects thereof are also the same.

X³ and X⁴ each independently represent an iodine atom, *—O—S(═O)₂R^{f} a bromine atom, or a chlorine atom, and an iodine atom, a bromine atom, or a chlorine atom is preferable and a bromine atom is more preferable.

### Step 1

Hereinafter, Step 1 will be described in detail. Step 1 is a step of reacting the compound represented by Formula (3a) with the compound represented by Formula (A) to obtain a compound represented by Formula (3b), which has a protective group represented by SiR^{Y1}₃.

In Formula (A), L¹ represents a leaving group. Examples of the leaving group include a halogen atom and *—O—S(═O)₂R^{f}. R has the same meaning as R^{f} in Formula (2). Among these, L¹ is preferably a bromine atom or a chlorine atom.

In Formula (A), R^{Y1} represents an aliphatic hydrocarbon group which may have a substituent, an aromatic ring group which may have a substituent, or an aliphatic heterocyclic group which may have a substituent. A plurality of R^{Y1}'s may be the same as or different from each other.

Among these, as R^{Y1}, a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, or an aromatic ring group is preferable.

In Step 1, typically, the hydrogen atom on N in the compound represented by Formula (3a) is converted into lithium using an organolithium reagent, and then the compound represented by Formula (A) is reacted. The reaction conditions are not particularly limited as long as the conditions are generally for lithiation, and specific examples of the organolithium reagent are as described above.

### Step 2

Next, Step 2 will be described in detail. Step 2 is a step of reacting the compound represented by Formula (3b) obtained in Step 1 with a metalating reagent, then reacting the compound with a formylating agent, and further deprotecting the protective group to obtain a compound represented by Formula (3c) described later.

In Step 2, known metalating reagents can be used as the metalating reagent, and for example, the reagents exemplified in Step P2A can be used. The reaction conditions are not particularly limited, and known reaction conditions can be applied.

As the formylating agent, a known agent can be used, and for example, N,N-disubstituted formamide and an orthoformic ester, which are exemplified in Step P2A, can be used. Known conditions can also be adopted for the reaction conditions, and examples thereof include the conditions exemplified in Step P2A.

Examples of the method of deprotecting the protective group represented by SiR^{Y1}₃ include a method of reacting an appropriate de-silylating agent depending on the compound represented by Formula (A) used.

As the de-silylation agent is not particularly limited, a known de-silylation agent can be used, but examples thereof include water, an acid, a base, and a fluoride ion.

It is noted that depending on the structure of the compound represented by Formula (A), the silyl protective group derived from the compound represented by Formula (A) is likely to be deprotected, and deprotection may occur due to a post-step (a liquid separation step, a column purification step, and the like) after the completion of the reaction, moisture in the air, or the like.

Even in such a case, as long as the compound represented by Formula (3c) can be obtained from the compound represented by Formula (3b), it is included in the scope of the present invention.

It is noted that Step 1 and Step 2 may be performed in one pot depending on the reaction conditions.

### Step 3

Next, Step 3 will be described in detail. Step 3 is a step of obtaining the compound represented by Formula (3) by reacting the compound represented by Formula (3c), which is obtained in Step 2, with the compound represented by Formula (C) and introducing a group derived from the compound represented by Formula (C) into N in Formula (3c).

In Formula (C), L² represents a leaving group. Examples of the leaving group include *—O—(C═O)R⁶, a halogen atom, and *—O—S(═O)₂R^{f}. R has the same meaning as R in Formula (A). R⁶ has the same meaning as R⁶ in Formula (3), and is preferably a methyl group or an ethyl group.

As L², *—O—(C═O)R⁶ or a halogen atom is preferable, *—O—(C═O)R⁶, a chlorine atom, or a bromine atom is more preferable, and *—O—(C═O)R⁶ or a chlorine atom is still more preferable.

Specific examples of the compound represented by Formula (3) will be shown below, but the present invention is not limited thereto.

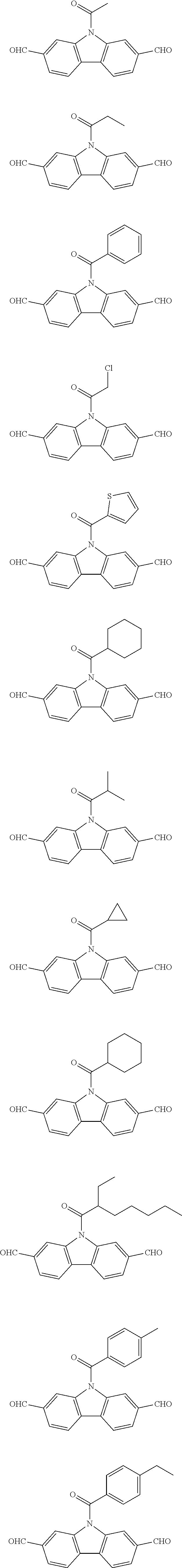

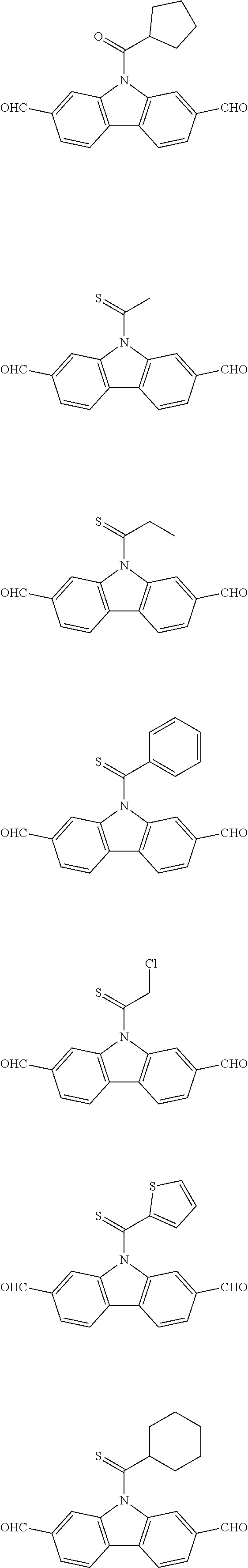

### EXAMPLES

Hereinafter, the present invention will be described in more detail based on Examples.

The materials, the amounts and proportions of the materials used, the details of treatments, the procedure of treatments, and the like shown in the following Examples can be appropriately modified as long as the gist of the present invention is maintained. Therefore, the scone of the present invention should not be construed as being limited to Examples shown below.

### [Materials Used for Photoelectric Conversion Film]

Various components contained in the photoelectric conversion film are shown below.

### Synthesis of Compound 1 (Synthesis Example 1 of Specific Compound)

Compound 1 was synthesized according to the following scheme.

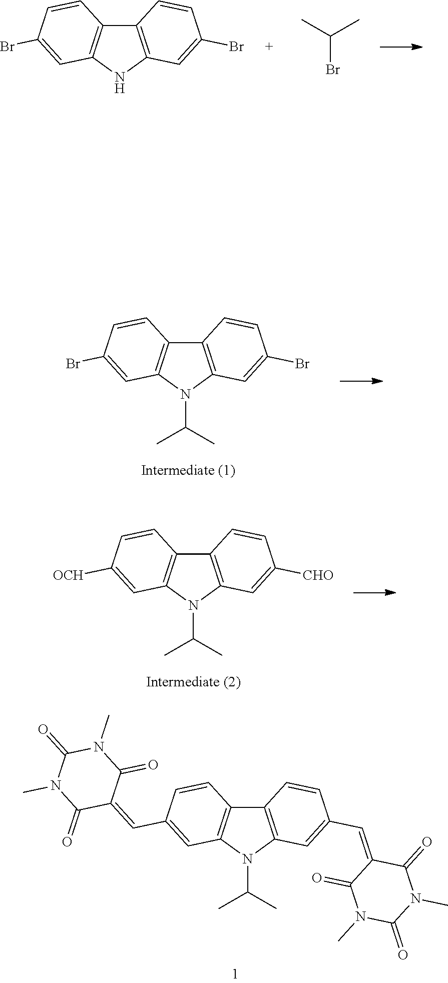

### <Synthesis of Intermediate (1)>

Intermediate (1) was synthesized using 2,7-dibromocarbazole and 2-bromopropane as starting materials, according to the method described in Marciniec et al., Org. Biomol. Chem., 2016, 14, 9406.

### <Synthesis of Intermediate (2)>

Intermediate (1) (7.0 g) and tetrahydrofuran (THF, 180 mL) were placed in a 300 mL three-neck flask, and the temperature was lowered to −78° C. under a nitrogen atmosphere.

A hexane solution (2.8 M, manufactured by FUJIFILM Wako Pure Chemical Corporation, 27 mL) of n-butyllithium was added thereto, the mixture was stirred for 1 hour, and then N,N-dimethylformamide (DMF, 15 mL) was added thereto and stirred at room temperature for 1 hour.

The obtained reaction solution was sequentially washed with a saturated aqueous ammonium chloride solution and a saturated saline solution, and then the organic phase was recovered. The obtained organic phase was dried using sodium sulfate, and the solvent was further removed. The obtained crude product was purified by silica gel column chromatography (eluent: hexane/dichloromethane=1/1), thereby obtaining 2.4 g of Intermediate (2) (yield of 40%).

### <Synthesis of Compound 1>

Intermediate (2) (500 mg), 1,3-dimethylbarbituric acid (manufactured by Tokyo Chemical Industry Co., Ltd., 765 mg), acetic acid (20 mL), and piperidine (manufactured by FUJIFILM Wako Pure Chemical Corporation, 37 μL) were placed in a 100 mL eggplant flask and reacted at 100° C. for 17 hours under a nitrogen atmosphere.

After completion of the reaction, the precipitated solid was separated by filtration, and the crude product was recrystallized from dichloromethane and methanol. The obtained solid was sublimated and purified to obtain 767 mg (yield of 75%) of Compound 1.

The ¹H-nuclear magnetic resonance (NMR) data of Compound 1 obtained are shown below.

¹H-NMR (CDCl₃): δ (ppm)=8.94 (2H, s), 8.79 (2H, s), 8.18 (2H, d), 7.86 (2H, dd), 5.15 (1H, sep), 3.47 (6H, s), 3.45 (6H, s), 1.84 (6H, d)

### Synthesis of Compound 3 (Synthesis Example 2 of Specific Compound)

Compound 3 was synthesized according to the following scheme.

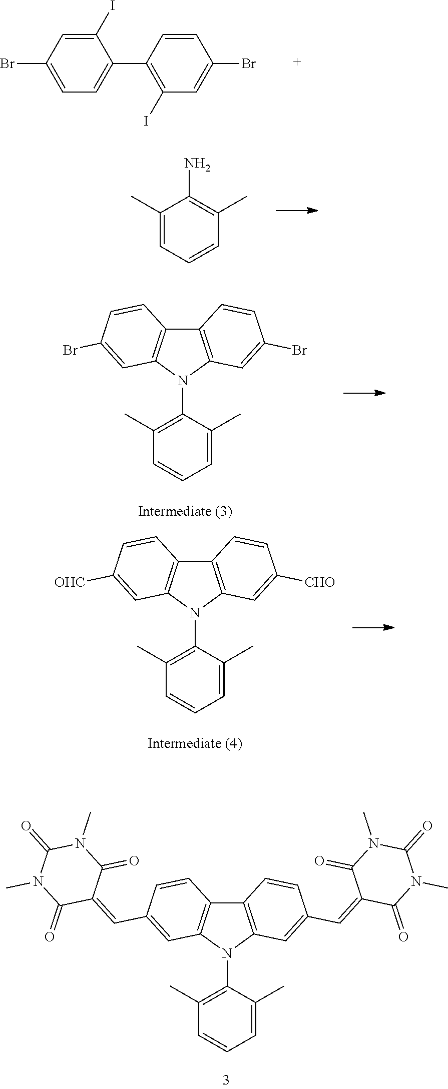

### <Synthesis of Intermediate (3)>

4,4′-dibromo-2,2′-diiodobiphenyl (manufactured by Combi-Blocks, Inc., 18 g), 2,6-dimethylaniline (manufactured by FUJIFILM Wako Pure Chemical Corporation, 5.8 g), potassium-t-butoxide (manufactured by FUJIFILM Wako Pure Chemical Corporation, 11 g), toluene (750 mL), and tetrakis(triphenylphosphine)palladium (0) (manufactured by Tokyo Chemical Industry Co., Ltd., 3.7 g) were placed in a 2 L three-neck flask, and the mixture was stirred at 100° C. for 39 hours under a nitrogen atmosphere.

The reaction solution was cooled to room temperature, and then filtered through Celite to remove insoluble components, and the solvent was removed from the obtained filtrate. The obtained crude product was purified by silica gel column chromatography (eluent: hexane/toluene=99/1) to obtain 3.7 g of Intermediate (3) (yield of 25%).

The ¹H-NMR data of Intermediate (3) obtained are shown below.

¹H-NMR (CDCl₃): δ=7.97 (d, 2H), 7.39 (dd, 2H), 7.38 (d, 1H), 7.29 (d, 2H), 7.07 (d, 2H), 1.86 (s, 6H)

### <Synthesis of Intermediate (4)>

Intermediate (4) was synthesized in the same procedure as in <Synthesis of Intermediate (2)>.

The ¹H-NMR data of Intermediate (4) obtained is shown below.

¹H-NMR (CDCl₃): δ=10.1 (s, 2H), 8.37 (d, 2H), 7.87 (dd, 2H), 7.54 (s, 2H), 7.43 (d, 1H), 7.32 (d, 2H), 1.85 (s, 6H)

### <Synthesis of Compound 3>

Intermediate (4) (250 mg), 1,3-dimethylbarbituric acid (310 mg), acetic acid (25 mL), and piperidine (15 L) were placed in a 100 mL eggplant flask and reacted at 100° C. for 2 hours under a nitrogen atmosphere.

The precipitated solid was separated by filtration, and the crude product was recrystallized from dichloromethane and methanol. The obtained solid was sublimated and purified to obtain 250 mg (yield of 54%) of Compound 3.

The ¹H-NMR data of Compound 1 obtained are shown below.

¹H-NMR (CDCl₃): δ=8.65 (2H, s), 8.26 (2H, d), 8.17 (2H, dd), 7.70 (2H, s), 7.42 (1H, dd), 2.11 (2H, d), 3.41 (6H, s), 3.36 (6H, s), 1.92 (6H, s)

### Comparative Synthesis Example 1 (Comparative Synthesis Example of Intermediate (3))

As shown in the following scheme, 2,7-dibromocarbazole (100 mg), 2-fluoro-1,3-dimethylbenzene (76 mg), cesium carbonate (201 mg), and DMF (1.5 mL) were added to an eggplant flask, and the mixture was heated at 100° C. and stirred for 1 hour, but Intermediate (3) which was a target was not obtained.

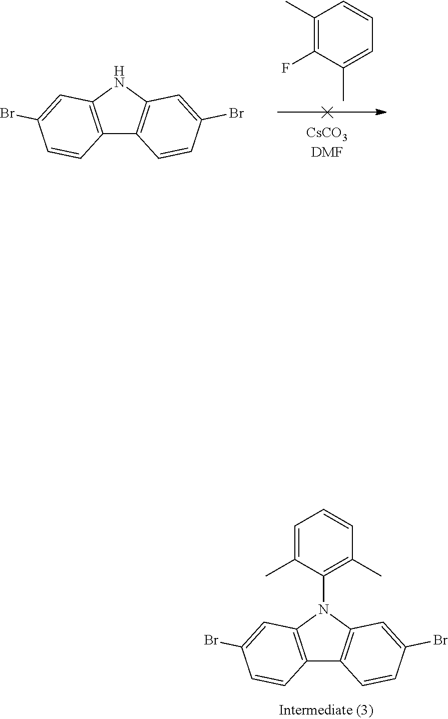

### Synthesis of Compound 24 (Synthesis Example 3 of Specific Compound)

The compound 24 was synthesized according to the following scheme.

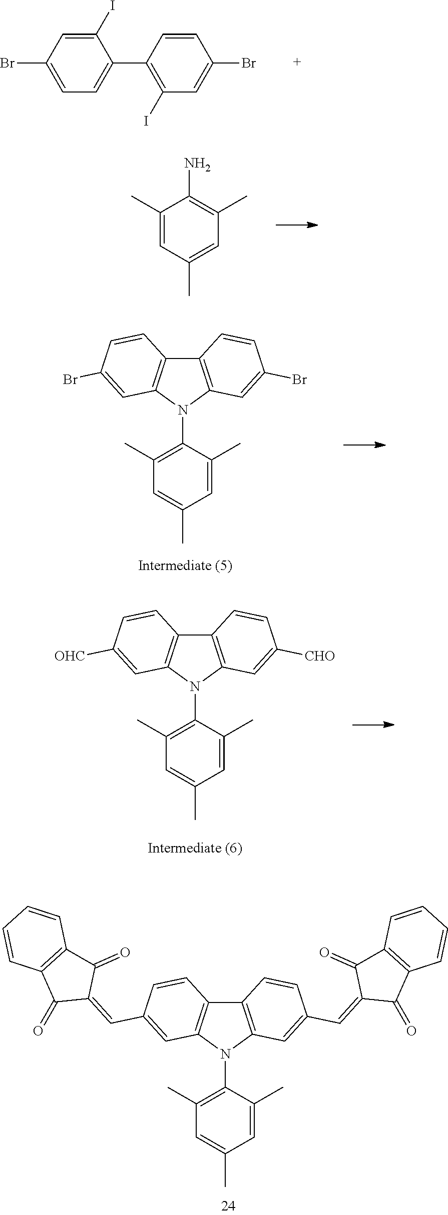

### <Synthesis of Intermediate (5)>

Intermediate (5) was synthesized in the same procedure as <Synthesis of Intermediate (3)>, except that 2,6-dimethylaniline was changed to 2,4,6-trimethylaniline in <Synthesis of Intermediate (3)>.

The ¹H-NMR data of Intermediate (5) obtained are shown below.

¹H-NMR (CDCl₃): δ=7.96 (d, 2H), 7.38 (dd, 2H), 7.09 (d, 2H), 7.08 (d, 2H), 2.43 (s, 3H), 1.81 (s, 6H)

### <Synthesis of Intermediate (6)>

Intermediate (6) was synthesized in the same procedure as in <Synthesis of Intermediate (2)> using Intermediate (5) instead of Intermediate (3).

The ¹H-NMR data of Intermediate (6) obtained are shown below.

¹H-NMR (CDCl₃): δ=10.1 (s, 2H), 8.36 (d, 2H), 7.86 (dd, 2H), 7.55 (d, 2H), 7.13 (d, 2H), 2.45 (s, 3H), 1.80 (s, 6H)

### <Synthesis of Compound 24>

Compound 24 was synthesized in the same procedure as <Synthesis of Compound 3>, except that 1,3-dimethylbarbituric acid was changed to 1,3-indandione in the synthesis of <Synthesis of Compound 3> in which Intermediate (6) was used instead of Intermediate (4).

The ¹H-NMR data of the obtained compound 24 are shown below.

¹H-NMR (CDCl₃): δ=8.63 (dd, 2H), 8.34 (d, 2H), 8.02 (m, 8H), 7.80 (m, 4H), 7.21 (s, 2H), 2.51 (s, 3H), 1.91 (s, 6H)

### Synthesis of Compound 34 (Synthesis Example 4 of Specific Compound)

The compound 34 was synthesized according to the following scheme.

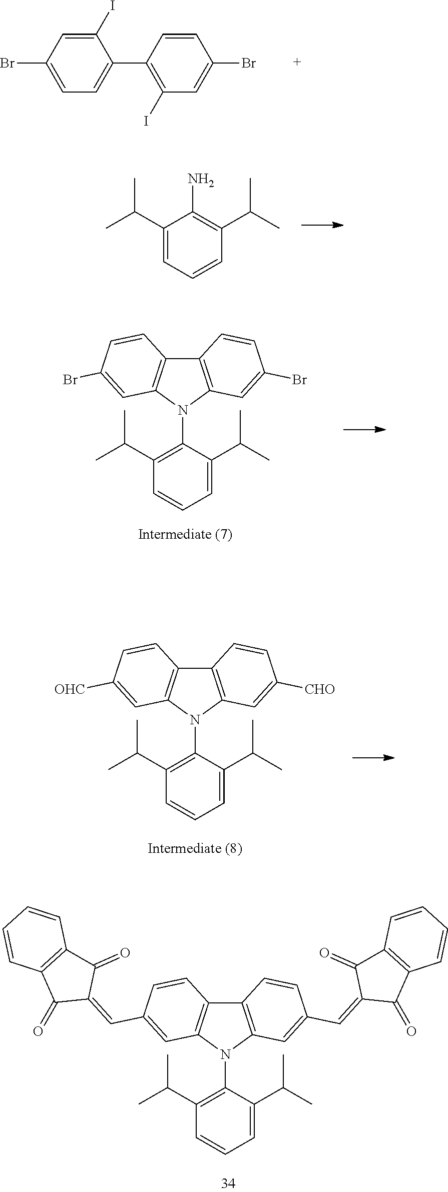 [SMILES: Brc1ccc(-c2ccc(Br)cc2I)c(I)c1 ; CC(C)c1cccc(C(C)C)c1N ; CC(C)c1cccc(C(C)C)c1-n1c2cc(Br)ccc2c2ccc(Br)cc21 ; CC(C)c1cccc(C(C)C)c1-n1c2cc(C=O)ccc2c2ccc(C=O)cc21 ; CC(C)c1cccc(C(C)C)c1-n1c2cc(C=C3C(=O)c4ccccc4C3=O)ccc2c2ccc(C=C3C(=O)c4ccccc4C3=O)cc21]

### <Synthesis of Intermediate (7)>

Intermediate (7) was synthesized in the same procedure as <Synthesis of Intermediate (3)>, except that 2,6-dimethylaniline was changed to 2,6-diisopropylaniline.

The ¹H-NMR data of Intermediate (7) obtained are shown below.

¹H-NMR (CDCl₃): δ=7.97 (d, 2H), 7.57 (t, 1H), 7.39 (d, 2H), 7.39 (dd, 2H), 7.07 (d, 2H), 2.14 (sept, 2H), 0.99 (d, 12H)

### <Synthesis of Intermediate (8)>

Intermediate (8) was synthesized in the same procedure as in <Synthesis of Intermediate (2)> using Intermediate (7) instead of Intermediate (1).

The ¹H-NMR data of Intermediate (8) obtained are shown below.

¹H-NMR (CDCl₃): δ=10.1 (s, 2H), 8.37 (d, 2H), 7.87 (dd, 2H), 7.62 (t, 1H), 7.55 (d, 2H), 7.43 (d, 2H), 2.12 (sept, 2H), 0.97 (d, 12H)

### <Synthesis of Compound 34>

Compound 34 was synthesized in the same procedure as <Synthesis of Compound 3>, except that 1,3-dimethylbarbituric acid was changed to 1,3-indandione in the synthesis of <Synthesis of Compound 3> in which Intermediate (8) was used instead of Intermediate (4).

The ¹H-NMR data of Compound 34 obtained are shown below.

¹H-NMR (CDCl₃): δ=8.70 (dd, 2H), 8.36 (d, 2H), 8.02-7.96 (m, 6H), 7.82-7.78 (m, 4H), 7.69 (t, 1H), 7.50 (d, 2H), 2.22 (sept, 2H), 1.02 (d, 12H)

In Synthesis Examples 1 to 4, the specific compound is synthesized from Intermediate A in which the group represented by R⁴ and the group represented by R⁵ are a formyl group (hereinafter, also referred to as a “formyl intermediate”).

On the other hand, Intermediate A in which the group represented by R⁴ and the group represented by R⁵ are *—Sn(R^{Sn})₃, *—B(R^{B1})₂, or *—B⁻(R^{B2})₃M⁺ (hereinafter, also referred to as “other intermediates”) as a raw material can be converted into the above-described formyl intermediate, and then the specific compound can be synthesized with reference to Synthesis Examples 1 to 4.

Examples of the synthesis of other intermediates are shown below.

### <Synthesis of Sn Body>

Intermediate A in which the group represented by R⁴ and the group represented by R⁵ are *—Sn(R^{Sn})₃ (hereinafter, also referred to as “Sn body”) was synthesized according to the following scheme.

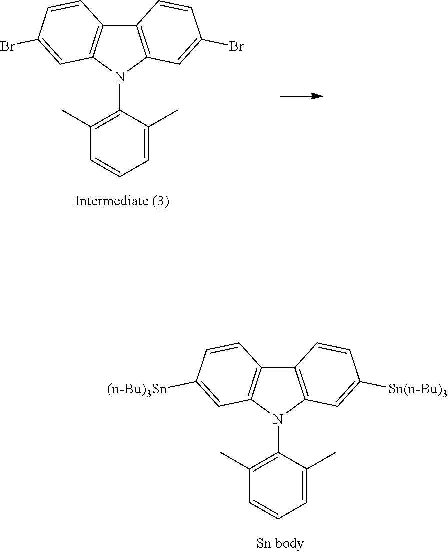

Intermediate (3) (3.0 g) and THF (77 mL) were placed in a three-neck flask, and the temperature was lowered to −78° C. under a nitrogen atmosphere. A hexane solution (2.8 M, 12 mL) of n-butyllithium was added thereto, the mixture was stirred for 1 hour, and then tributyl tin chloride (manufactured by Tokyo Chemical Industry Co., Ltd., 9.5 mL) was added thereto, and the mixture was stirred at room temperature for 1 hour.

The obtained reaction solution was sequentially washed with a saturated aqueous ammonium chloride solution and a saturated saline solution, and then the organic phase was recovered. The obtained organic phase was dried using sodium sulfate, and the solvent was further removed.

The obtained crude product was purified by silica gel column chromatography (NH₂ column, eluent: hexane) to obtain 5.0 g (yield of 85%) of an Sn body.

The structure of the Sn body was confirmed by liquid chromatography-mass spectrometry (LC-MS).

LC-MS (Sn body): 851.3 (M⁺)

### <Synthesis of B Body>

An intermediate A in which the group represented by R⁴ and the group represented by R⁵ are *—B(R^{B1})₂ or *—B⁻(R^{B2})₃M⁺ (hereinafter, also referred to as a “B body”) was synthesized according to the following scheme.

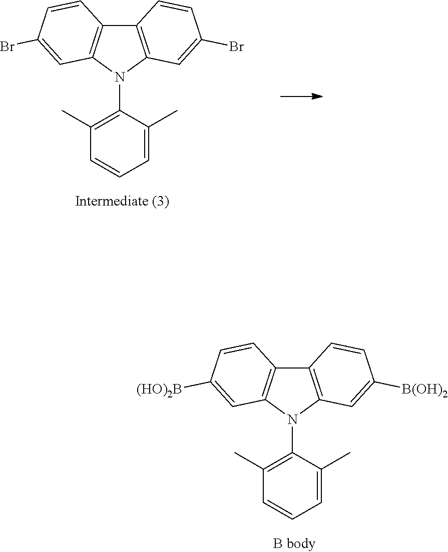 [SMILES: Cc1cccc(C)c1-n1c2cc(Br)ccc2c2ccc(Br)cc21 ; Cc1cccc(C)c1-n1c2cc(B(O)O)ccc2c2ccc(B(O)O)cc21]

Intermediate (3) (1.0 g) and THE (100 mL) were placed in a three-neck flask, and the temperature was lowered to −100° C. under a nitrogen atmosphere. A hexane solution (2.8 M, 1.8 mL) of n-butyllithium was added thereto, the mixture was stirred for 1.5 hours, then trimethoxyborane (manufactured by Tokyo Chemical Industry Co., Ltd., 0.58 g) was added thereto, and the mixture was allowed to react at room temperature for 18 hours.

100 mL of ethyl acetate and hydrochloric acid (3M, 100 mL) were added to the obtained reaction solution, the mixture was stirred, and then the water phase was removed. The obtained organic phase was washed with water, then dried with sodium sulfate, and the solvent was further removed.

The obtained crude product was purified by silica gel column chromatography (eluent: toluene) to obtain 0.38 g (yield of 45%) of a B body.

The structure of the B body was confirmed by LC-MS.

LC-MS (B body): 359.0 (M⁺)

### Synthesis of Compound 36 (Synthesis Example 5 of Specific Compound)

The compound 36 was synthesized according to the following scheme.

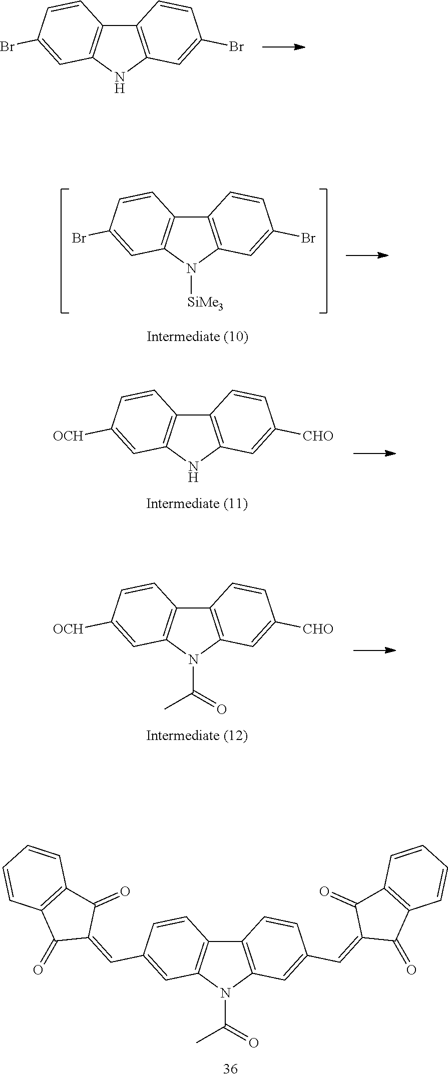

### <Synthesis of Intermediate (11)>

2,7-Dibromocarbazole (5.0 g) and THF (280 mL) were placed in a three-neck flask, and the temperature was lowered to 0° C. under a nitrogen atmosphere. A hexane solution (2.7 M, manufactured by FUJIFILM Wako Pure Chemical Corporation, 5.8 mL) of n-butyllithium was added thereto, the mixture was stirred for 5 minutes, and then trimethylchlorosilane (2.0 mL) was added thereto. After stirring at room temperature for 30 minutes, disappearance of 2,7-dibromocarbazole and generation of Intermediate (10) were confirmed by LC-MS.

A hexane solution (2.7 M, 23 mL) of n-butyllithium was added dropwise to the reaction solution at −78° C., DMF (12 mL) was added dropwise thereto, and the mixture was stirred at room temperature for 10 minutes. Ethyl acetate (280 mL) was added to the obtained reaction solution, the mixture was washed with water and saline in order, and then the organic phase was recovered. The obtained organic phase was dried using sodium sulfate, and the solvent was further removed. The obtained crude product was purified by silica gel column chromatography (eluent: hexane/ethyl acetate=1/1), thereby obtaining 1.4 g of Intermediate (11) (yield of 40%).

The ¹H-NMR data of Intermediate (11) obtained are shown below.

¹H-NMR (DMSO-d₆): δ=12.1 (s, 1H), 10.2 (s, 2H), 8.44 (d, 2H), 8.15 (d, 2H), 7.78 (dd, 2H).

### <Synthesis of Intermediate (12)>

Intermediate (11) (1.3 g), 4-dimethylaminopyridine (manufactured by FUJIFILM Wako Pure Chemical Corporation, 0.29 g), THF (45 mL), triethylamine (2.5 mL), and acetic anhydride (manufactured by FUJIFILM Wako Pure Chemical Corporation, 2.2 mL) were added to a three-neck flask, and the mixture was stirred at room temperature for 1 hour.

300 mL of chloroform was added to the reaction solution, the mixture was washed with a saturated aqueous ammonium chloride solution and saline, and then the organic phase was recovered. The obtained organic phase was dried using sodium sulfate, and the solvent was further removed. The obtained crude product was purified by silica gel column chromatography (eluent: chloroform/ethyl acetate=7/3), thereby obtaining 0.74 g of Intermediate (12) (yield of 48%).

The ¹H-NMR data of Intermediate (12) obtained are shown below.

¹H-NMR (CDCl₃): δ=10.2 (s, 2H), 8.81 (s, 2H), 8.24 (d, 2H), 8.01 (d, 2H), 3.04 (s, 3H).

### Comparative Synthesis Example 2 (Comparative Synthesis Example of Intermediate (12))

The synthesis of Intermediate (12) was attempted according to the following scheme.

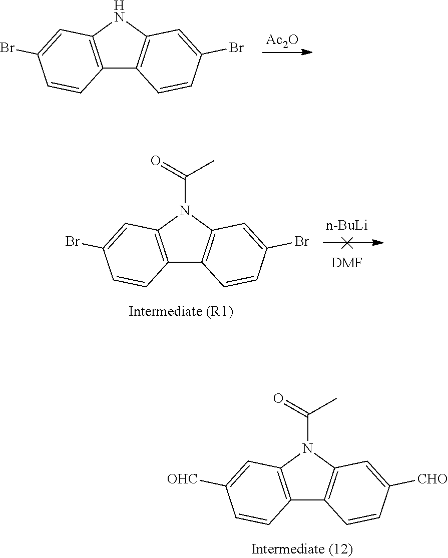

Intermediate (R1) was synthesized in the same procedure as in <Synthesis of Intermediate (12)> using 2,7-dibromocarbazole as a starting material.

Next, Intermediate (R1) (200 mg), THF (5.5 mL), and n-butyllithium (2.7 M, 0.5 mL) were placed in a three-neck flask, and the mixture was stirred at −90° C. for 1 hour under a nitrogen atmosphere. DMF (0.4 mL) was added to the reaction solution, the temperature was raised to room temperature, then the product was confirmed by LC-MS, but Intermediate (12) which was a target was not obtained because the decomposition of the acetyl group and the like proceeded.

### Comparative Synthesis Example 3 (Comparative Synthesis Example of Intermediate (12))

As shown in the following scheme, the synthesis of Intermediate (12) by the same procedure as described above using 2,7-dibromocarbazole as a starting material, but a complex mixture was obtained and Intermediate (12) which was a target was not obtained.

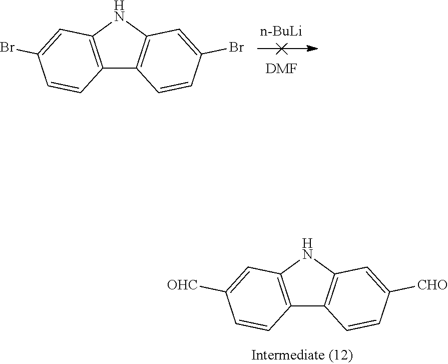

### <Synthesis of Compound 36>

Compound 36 was synthesized in the same procedure as <Synthesis of Compound 3>, except that 1,3-dimethylbarbituric acid was changed to 1,3-indandione in the synthesis of <Synthesis of Compound 3> in which Intermediate (12) was used instead of Intermediate (4).

Since Compound 36 has low solubility, the structure thereof was confirmed by laser desorption ionization mass spectrometry (LDI-MS).

LDI-MS (Compound (36)): 521.1 (M⁺)

The specific compounds and comparative compounds used in the photoelectric conversion film other than Compound 1, Compound 3, Compound 24, Compound 34, and Compound 36 were synthesized with reference to Synthesis Examples 1 to 5.

Each material used for the photoelectric conversion film is shown below. Compounds 1 to 38 correspond to specific compounds, and Compounds C-1 to C-6 correspond to comparative compounds.

### [Specific Compound and Comparative Compound]

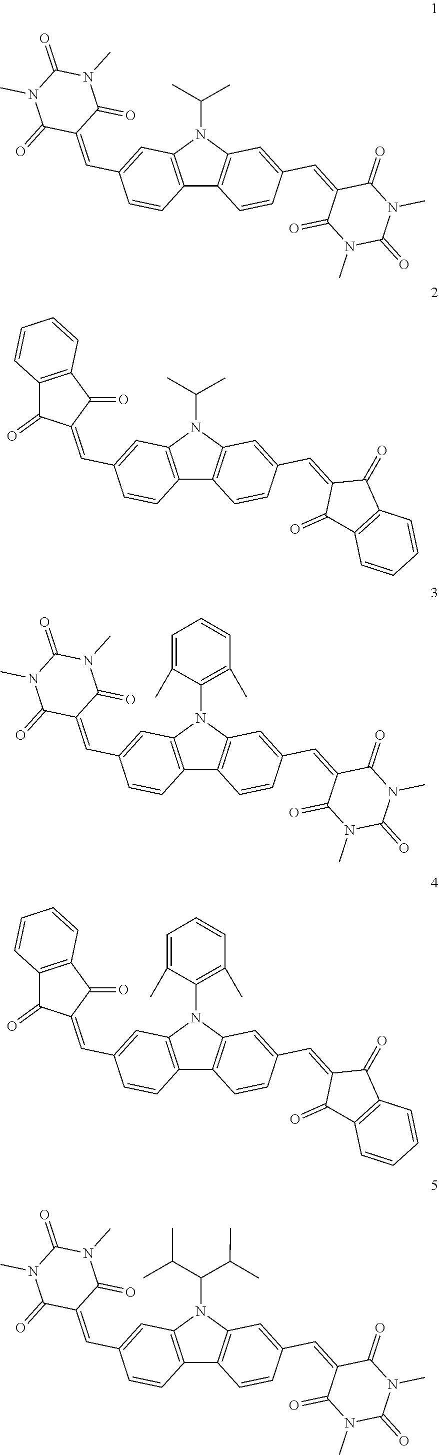

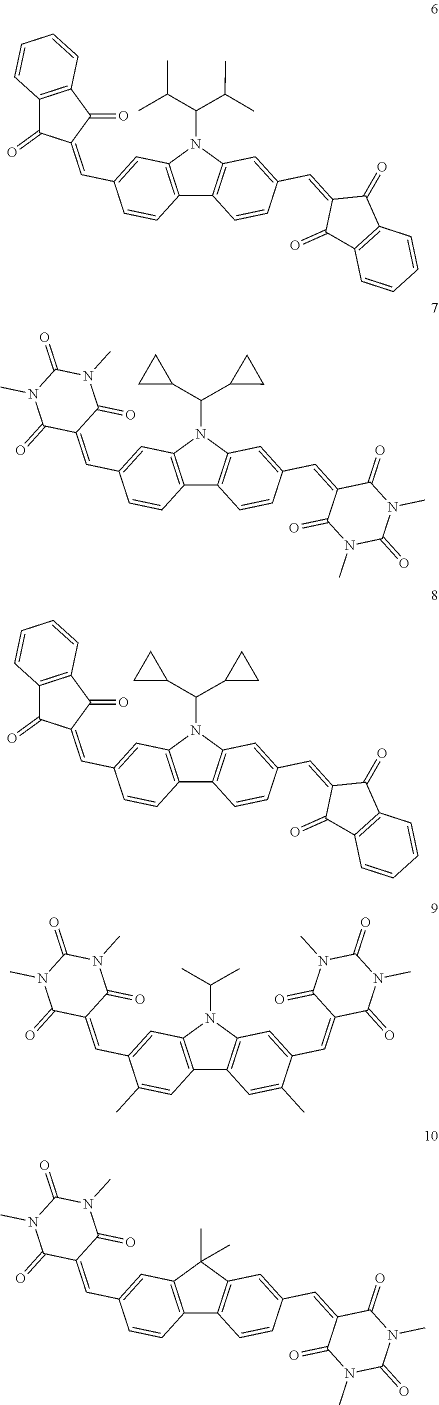

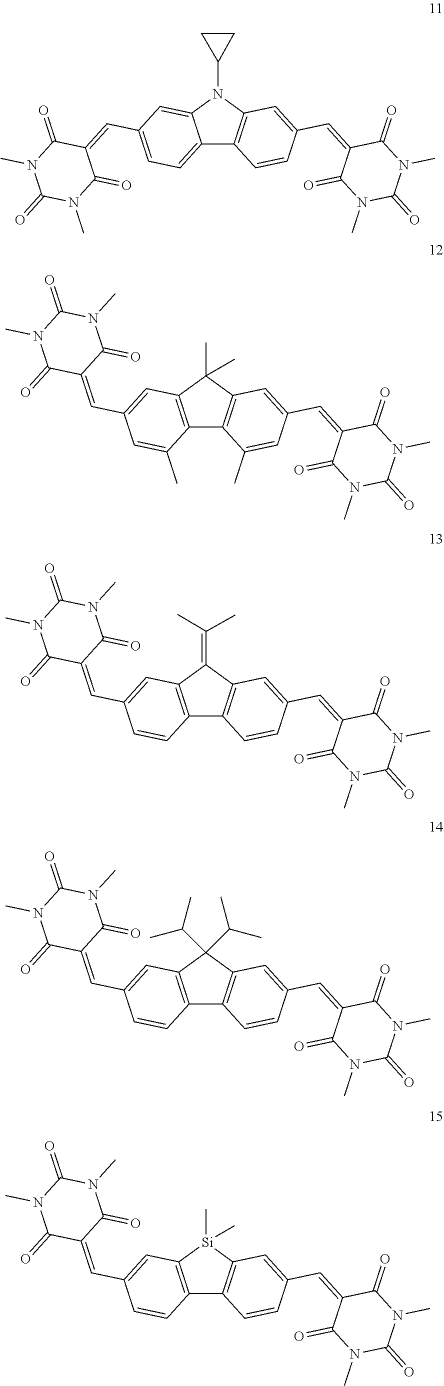

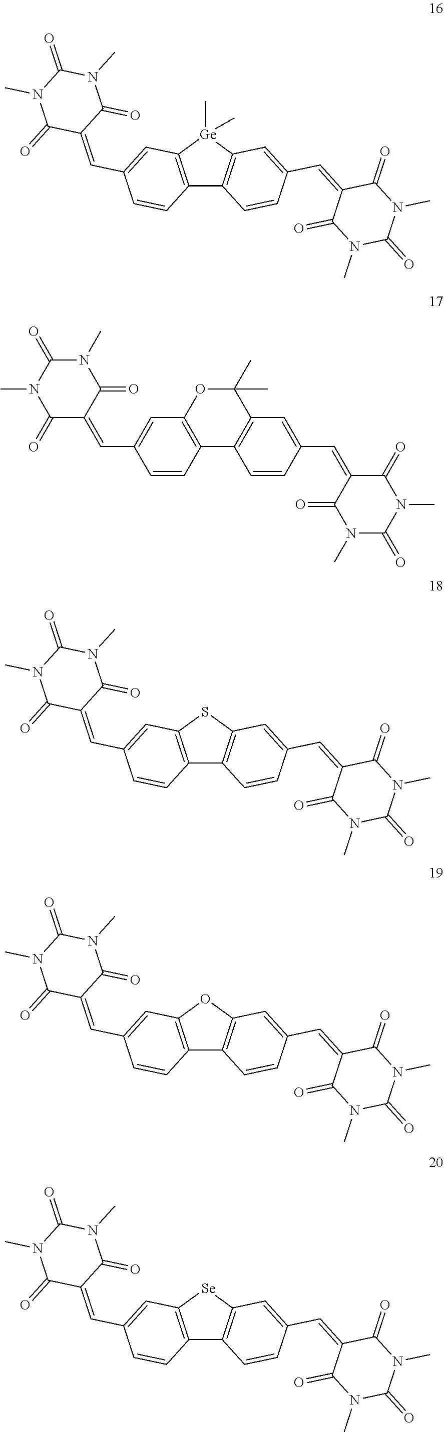

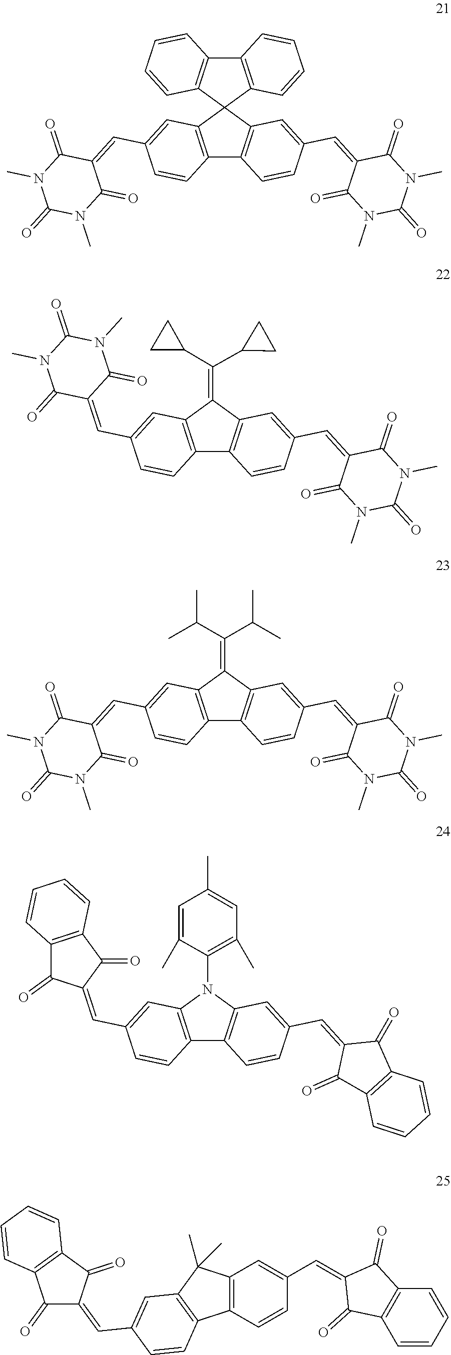

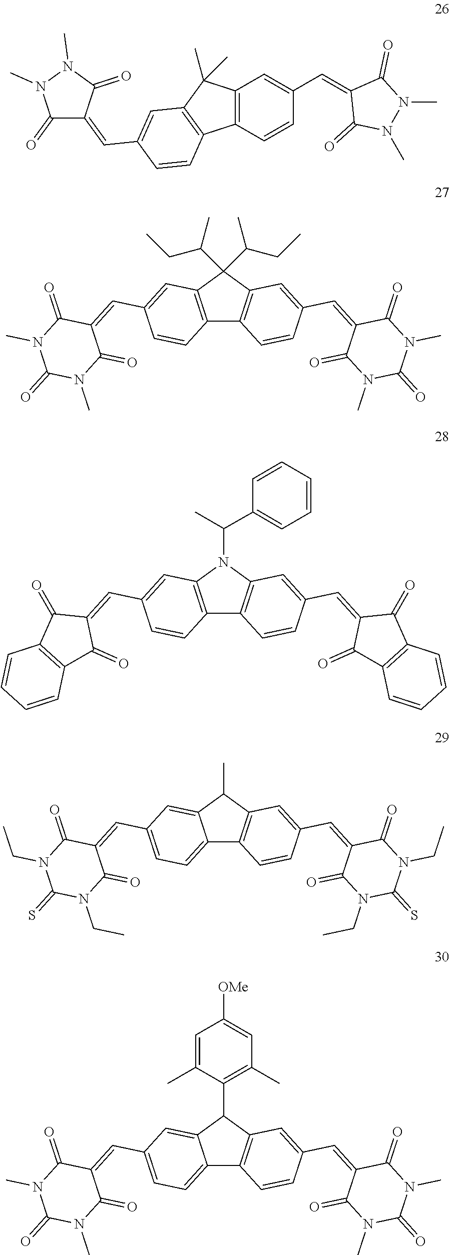

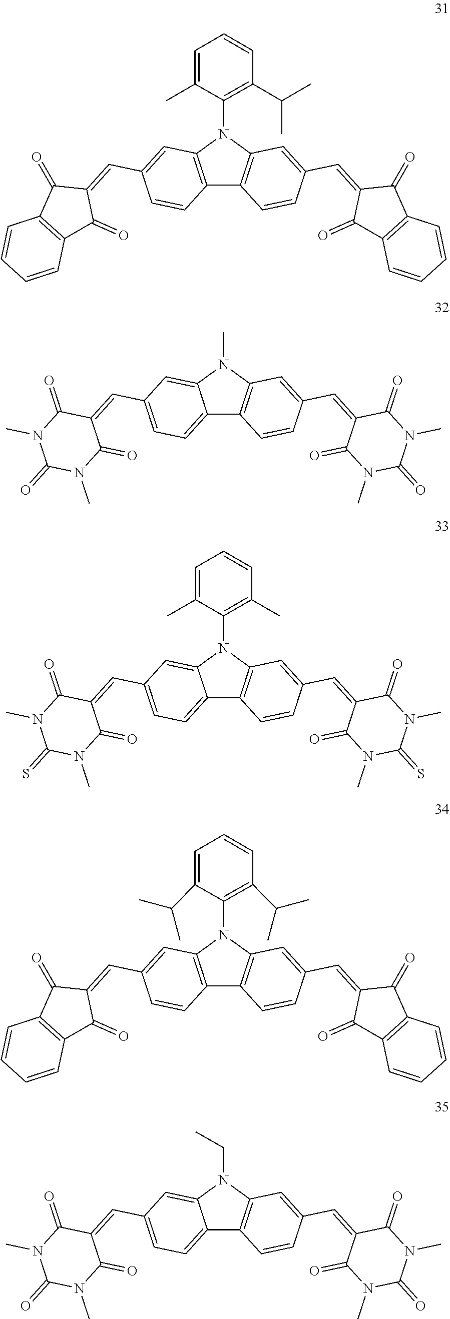

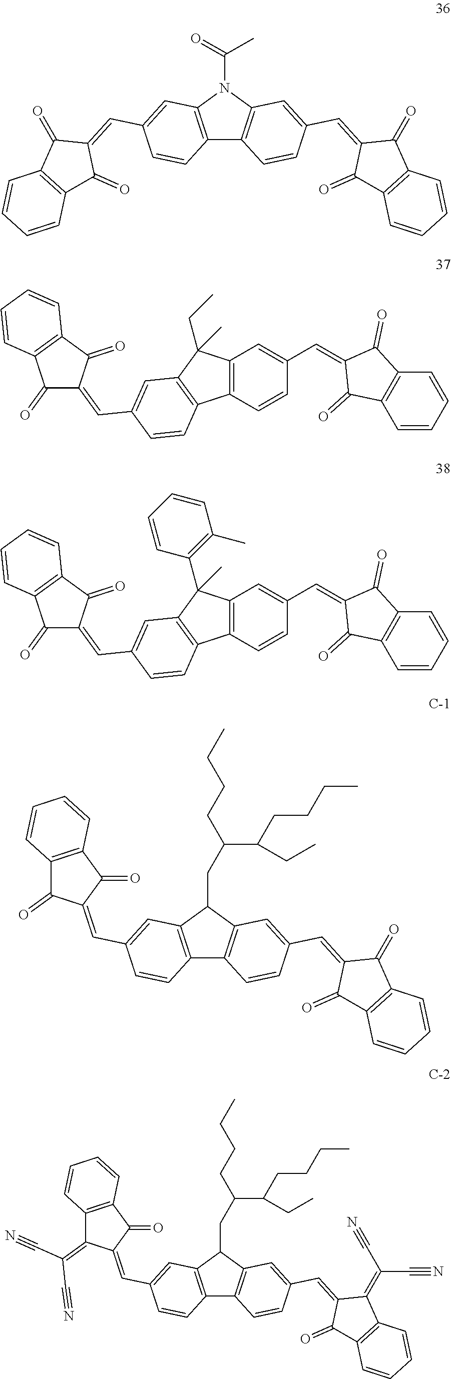 [SMILES: CC(=O)n1c2cc(C=C3C(=O)c4ccccc4C3=O)ccc2c2ccc(C=C3C(=O)c4ccccc4C3=O)cc21 ; CCC1(C)c2cc(C=C3C(=O)c4ccccc4C3=O)ccc2-c2ccc(C=C3C(=O)c4ccccc4C3=O)cc21 ; Cc1ccccc1C1(C)c2cc(C=C3C(=O)c4ccccc4C3=O)ccc2-c2ccc(C=C3C(=O)c4ccccc4C3=O)cc21 ; CCCCC(CC)C(CCCC)CC1c2cc(C=C3C(=O)c4ccccc4C3=O)ccc2-c2ccc(C=C3C(=O)c4ccccc4C3=O)cc21 ; CCCCC(CC)C(CCCC)CC1c2cc(/C=C3\C(=O)c4ccccc4C3=C(C#N)C#N)ccc2-c2ccc(/C=C3\C(=O)c4ccccc4C3=C(C#N)C#N)cc21]

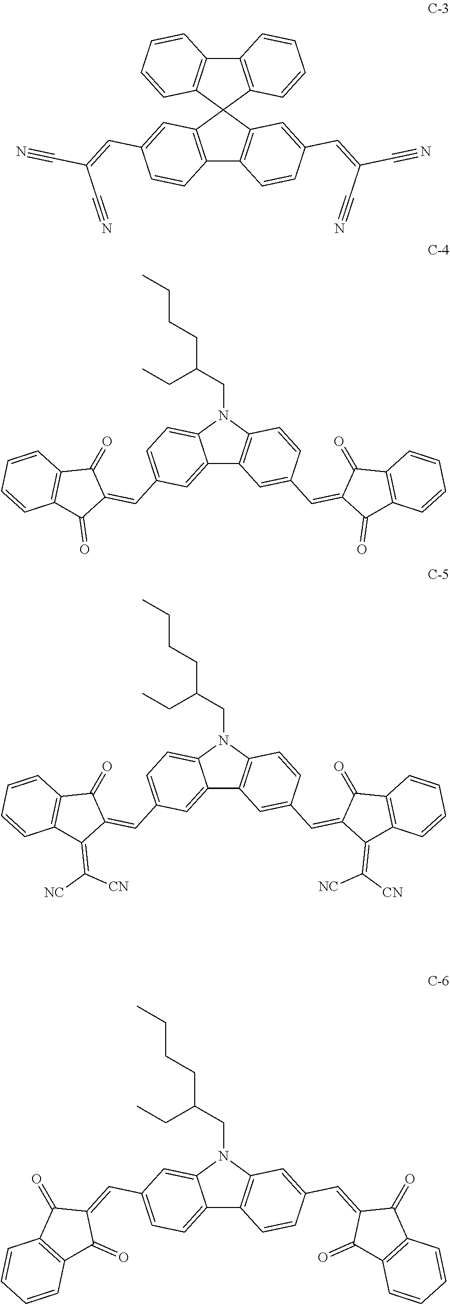 [SMILES: N#CC(C#N)=Cc1ccc2c(c1)C1(c3ccccc3-c3ccccc31)c1cc(C=C(C#N)C#N)ccc1-2 ; CCCCC(CC)Cn1c2ccc(C=C3C(=O)c4ccccc4C3=O)cc2c2cc(C=C3C(=O)c4ccccc4C3=O)ccc21 ; CCCCC(CC)Cn1c2ccc(/C=C3\C(=O)c4ccccc4C3=C(C#N)C#N)cc2c2cc(/C=C3\C(=O)c4ccccc4C3=C(C#N)C#N)ccc21 ; CCCCC(CC)Cn1c2cc(C=C3C(=O)c4ccccc4C3=O)ccc2c2ccc(C=C3C(=O)c4ccccc4C3=O)cc21]

[n-Type Organic Semiconductor]
C60: fullerene (C₆₀)
   [p-Type Organic Semiconductor]

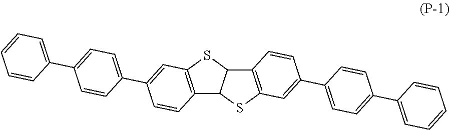 [SMILES: c1ccc(-c2ccc(-c3ccc4c(c3)SC3c5ccc(-c6ccc(-c7ccccc7)cc6)cc5SC43)cc2)cc1]

### [Coloring Agent]

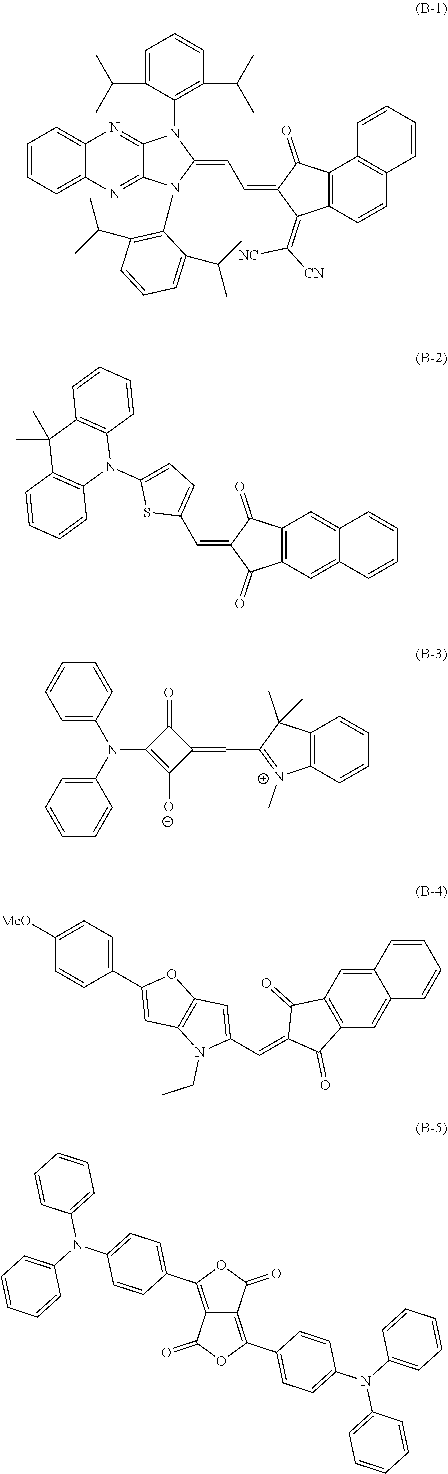 [SMILES: CC(C)c1cccc(C(C)C)c1N1C(=C/C=C2\C(=O)c3c(ccc4ccccc34)C2=C(C#N)C#N)N(c2c(C(C)C)cccc2C(C)C)c2nc3ccccc3nc21 ; CC1(C)c2ccccc2N(c2ccc(C=C3C(=O)c4cc5ccccc5cc4C3=O)s2)c2ccccc21 ; C[N+]1=C(C=C2C(=O)C(N(c3ccccc3)c3ccccc3)=C2[O-])C(C)(C)c2ccccc21 ; CCn1c(C=C2C(=O)c3cc4ccccc4cc3C2=O)cc2oc(-c3ccc(OC)cc3)cc21 ; O=C1OC(c2ccc(N(c3ccccc3)c3ccccc3)cc2)=C2C(=O)OC(c3ccc(N(c4ccccc4)c4ccccc4)cc3)=C12]

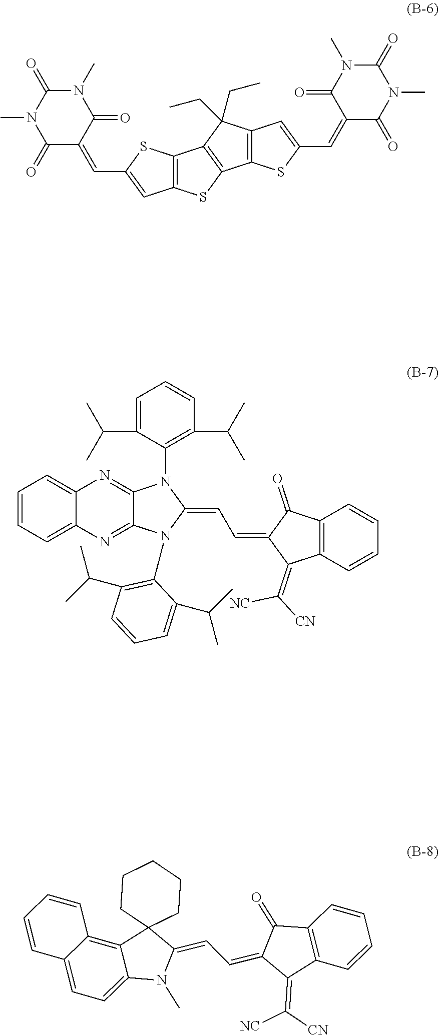

### [Evaluation]

A photoelectric conversion element was produced using the above-described material, and Test X and Test Y were performed.

### [Test X]

### <Production of Photoelectric Conversion Element>

A photoelectric conversion element having the form of FIG. 2 was produced using the various components shown above. Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, a compound (EB-1) was formed into a film on the lower electrode 11 by a vacuum thermal vapor deposition method to form the electron blocking film 16A (thickness: 30 nm).

Subsequently, in a state where the temperature of the glass substrate was controlled to 25° C., each specific compound or each comparative compound shown in Table 1, the n-type organic semiconductor (fullerene (C₆₀)), and the p-type organic semiconductor (Compound (P-1)) were co-vapor deposited on the electron blocking film 16A by a vacuum vapor deposition method, each to be 80 nm in terms of a single layer, thereby forming a film. As a result, the photoelectric conversion film 12 having a bulk hetero structure with a wavelength of 240 nm was formed. In this case, a film formation rate of the photoelectric conversion film 12 was set to 1.0 Å/sec.

Furthermore, a compound (EB-2) was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film) (thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method. The obtained laminate was heated in a glove box at 150° C. for 30 minutes to produce a photoelectric conversion element of each of Examples and Comparative Examples.

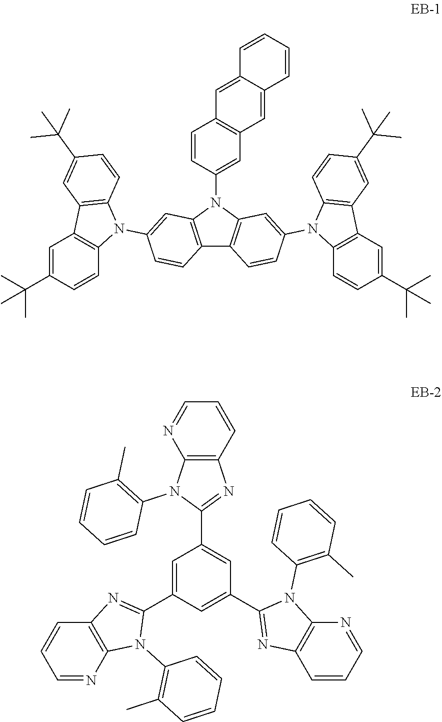 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### <Dark Current>

The dark current of each of the obtained photoelectric conversion elements was measured by the following method.

A voltage was applied to the lower electrode and the upper electrode of each of the photoelectric conversion elements to have an electric field strength of 2.5×10⁵ V/cm and current values (dark current) in a dark place were measured. As a result, it was confirmed that all of the photoelectric conversion elements had a dark current of 50 nA/cm² or less, which indicates that all of the photoelectric conversion elements had a sufficiently low dark current.

### <Quantum Efficiency>

The quantum efficiency of each of the obtained photoelectric conversion elements was measured by the following method.

A voltage was applied to each photoelectric conversion element such that the electric field strength was 2.0×10⁵ V/cm, and then light was emitted from the upper electrode (transparent conductive film) side to evaluate the quantum efficiency (photoelectric conversion efficiency) at a wavelength of 460 nm. The quantum efficiency was evaluated according to the following standard by using the value obtained in accordance with the following expression (S1).

In Formula (S1), for Examples and Comparative Examples shown in Table 1, Example 1-18 were adopted as the following standard examples.

quantum efficiency (relative ratio)=(quantum efficiency at wavelength of 460 nm in each of Examples or each of Comparative Examples)/(quantum efficiency at wavelength of 460 nm in the reference example) Expression (S1):

### (Evaluation Standard)

AA: quantum efficiency of 1.6 or more
A: quantum efficiency of 1.4 or more and less than 1.6
B: quantum efficiency of 1.2 or more and less than 1.4
C: quantum efficiency of 0.9 or more and less than 1.2
D: quantum efficiency of 0.5 or more and less than 0.9
E: quantum efficiency of less than 0.5

### <Response Speed (Responsiveness)>

The response speed of each obtained photoelectric conversion element was evaluated by the following method.

A voltage was applied to the photoelectric conversion element to have a strength of 2.0×10⁵ V/cm. Thereafter, a light emitting diode (LED) was turned on for an instant to emit light from the upper electrode (transparent conductive film) side, a photocurrent at this time at a wavelength of 460 nm was measured with an oscilloscope, a rise time until the signal strength rose from 0% to 97% was measured. The response rate was evaluated according to the following standard by using the value obtained in accordance with the following expression (S2).

In Formula (S2), for Examples and Comparative Examples shown in Table 1, Example 1-18 were adopted as the following standard examples.

relative response speed=(rise time at a wavelength of 460 nm in each of Examples or each of Comparative Examples)/(rise time at a wavelength of 460 nm in reference example) Expression (S2):

### (Evaluation Standard)

A: relative response speed of less than 0.5
B: relative response speed of 0.5 or more and less than 1.0
C: relative response speed of 1.0 or more and less than 1.5
D: relative response speed of 1.5 or more and less than 2.0
E: relative response speed of 2.0 or more

### <Electric Field Strength Dependence of Response Speed>

For each of the obtained photoelectric conversion elements, the electric field strength dependence of the response speed was evaluated by the following method.

The response speed at 7.5×10⁴ V/cm was measured by the same procedure as in the evaluation of the above-described <Response speed>, except that the voltage applied to each photoelectric conversion element was changed to 7.5×10⁴ V/cm. The electric field strength dependence of the response speed was evaluated according to the following standard using the value obtained according to the expression (S3).

In Expression (S3), each photoelectric conversion element in the numerator and the denominator are the same. For example, in the case of Example 1-1, the rise time of the photoelectric conversion efficiency at 7.5×10⁴ V/cm at a wavelength of 460 nm in Example 1-1 and the rise time of the photoelectric conversion efficiency at 2.0×10⁵ V/cm at a wavelength of 460 nm in Example 1-1 are compared.

electric field strength dependence of response speed=(rise time at 7.5×10⁴ V/cm at a wavelength of 460 nm in each of Examples or each of Comparative Examples)/(rise time at 2.0×10⁵ V/cm at a wavelength of 460 nm in each of Examples or each of Comparative Examples) Expression (S3):

(Evaluation standard)
A: electric field strength dependence of the response speed of less than 2.0
B: electric field strength dependence of the response speed of 2.0 or more and less than 3.0
C: electric field strength dependence of the response speed of 3.0 or more and less than 4.0
D: electric field strength dependence of the response speed of 4.0 or more and less than 5.0
E: electric field strength dependence of the response speed of 5.0 or more

### <Manufacturing Suitability>

The manufacturing suitability of each of the obtained photoelectric conversion elements was evaluated by the following method.

A photoelectric conversion element of each Example or each Comparative Example was produced as in the same procedure as <Production of photoelectric conversion element>, except that the film formation rate of the photoelectric conversion film 12 was changed to 3.0 Å/sec.

The photoelectric conversion element obtained in <Production of photoelectric conversion element> was defined as a photoelectric conversion element (A), the photoelectric conversion element obtained by setting the film formation rate of the photoelectric conversion film 12 to 3.0 Å/sec was defined as a photoelectric conversion element (B), and each quantum efficiency was determined as in the same procedure as in the evaluation of <Quantum efficiency>.

For the photoelectric conversion element having the same configuration of Examples or Comparative Examples, a relative ratio B/A of the quantum efficiency of the photoelectric conversion element (B) to the quantum efficiency of the photoelectric conversion element (A) (quantum efficiency of photoelectric conversion element (B)/quantum efficiency of photoelectric conversion element (A)) was calculated, and the manufacturing suitability was evaluated according to the following standard using the obtained value.

### (Evaluation Standard)

A: relative ratio B/A of 0.90 or more
B: relative ratio B/A of 0.85 or more and less than 0.90
C: relative ratio B/A of 0.80 or more and less than 0.85
D: relative ratio B/A of 0.75 or more and less than 0.80
E: relative ratio B/A of less than 0.75

Table 1 shows the evaluation results of Test X.

Each notation in Table 1 indicates the following.

In the column of “Substituent S definition 1”, in Formula (1), a case where the substituent selected from the substituent group S represented by R^{N} and R^{C1} to R^{C10} is a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{Ar1}, a group represented by Formula (S-3), or a group represented by Formula (S-4) is evaluated as “A”, and a case other than the above case is evaluated as “B”. In a case where X was a sulfur atom, an oxygen atom, or a selenium atom, it was evaluated as “-”.

In the column of “Formula (A-1)=Formula (A-2)”, a case where the group represented by Formula (A-1) is the group represented by Formula (A-2) is evaluated as “A”, and a case other than the above case is evaluated as “B”.

In the column of “Formula (A-1)=Formula (C-1) of Formula (C-2)”, a case where the group represented by Formula (A-1) in Formula (1) was the group represented by Formula (C-1) or Formula (C-2) was evaluated as “A”, and a case other than the above case were evaluated as “B”.

In the column of “X=having substituent S”, in Formula (1), a case where X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})— is evaluated as “A”, and a case other than the above case is evaluated as “B”.

In the column of “X═NR^{N} or CR₂”, in Formula (1), a case where X represents >NR^{N}, >CR^{C1}R^{C2}, or >C═CR^{C3}R^{C4} is evaluated as “A”, and a case other than the above case is evaluated as “B”.

In the column of “X═NR^{N}”, in Formula (1), a case where X represents NR^{N} is evaluated as “A”, and a case other than the above case is evaluated as “B”.

TABLE 1

| | | | | | | | | Evaluation | | | |
|---|---|---|---|---|---|---|---|---|---|---|---|
| | | | | | | | | | | Electric | |
| | | | | | | | | | | field | |
| | | | | | | | | | | strength | |
| | Specific compound or comparative compound | | | | | | | | | dependence | |
| | | | | Formula (A-1) = | X = having | X = | | | | of the | Manu- |
| | Com- | Substituent S | Formula (A-1) = | Formula (C-1) or | substituent | NR^{N} or | X = | Quantum | Response | response | facturing |
| Table 1 | pound | definition 1 | Formula (A-2) | Formula (C-2) | S | CR₂ | NR^{N} | efficiency | speed | speed | suitability |
| Example 1-1 | 1 | A | A | A | A | A | A | AA | B | A | A |
| Example 1-2 | 2 | A | A | A | A | A | A | AA | A | A | A |
| Example 1-3 | 3 | A | A | A | A | A | A | AA | B | A | A |
| Example 1-4 | 4 | A | A | A | A | A | A | AA | A | A | A |
| Example 1-5 | 5 | A | A | A | A | A | A | AA | B | A | A |
| Example 1-6 | 6 | A | A | A | A | A | A | AA | A | A | A |
| Example 1-7 | 7 | A | A | A | A | A | A | AA | B | A | A |
| Example 1-8 | 8 | A | A | A | A | A | A | AA | A | A | A |
| Example 1-9 | 9 | A | A | A | A | A | A | AA | B | A | A |
| Example 1-10 | 10 | A | A | A | A | A | B | A | B | A | A |
| Example 1-11 | 11 | A | A | A | A | A | A | AA | B | A | A |
| Example 1-12 | 12 | A | A | A | A | A | B | A | B | A | A |
| Example 1-13 | 13 | A | A | A | A | A | B | A | B | A | A |
| Example 1-14 | 14 | A | A | A | A | A | B | A | B | A | A |
| Example 1-15 | 15 | A | A | A | A | B | B | B | B | B | B |
| Example 1-16 | 16 | A | A | A | A | B | B | B | B | B | B |
| Example 1-17 | 17 | A | A | A | A | B | B | B | B | B | B |
| Example 1-18 | 18 | — | A | A | B | B | B | C | C | B | B |
| Example 1-19 | 19 | — | A | A | B | B | B | C | C | B | B |
| Example 1-20 | 20 | — | A | A | B | B | B | C | C | B | B |
| Example 1-21 | 21 | A | A | A | A | A | B | B | B | A | A |
| Example 1-22 | 22 | A | A | A | A | A | B | A | B | A | A |
| Example 1-23 | 23 | A | A | A | A | A | B | A | B | A | A |
| Example 1-24 | 24 | A | A | A | A | A | A | AA | A | A | A |
| Example 1-25 | 25 | A | B | B | A | A | B | C | C | B | A |
| Example 1-26 | 26 | A | A | B | A | A | B | B | C | B | A |
| Example 1-27 | 27 | B | A | A | A | A | B | B | B | B | B |
| Example 1-28 | 28 | B | A | A | A | A | A | A | A | A | A |
| Example 1-29 | 29 | A | A | A | A | A | A | AA | B | A | A |
| Example 1-30 | 30 | A | A | A | A | A | A | AA | B | A | A |
| Example 1-31 | 31 | A | A | A | A | A | A | AA | A | A | A |
| Example 1-32 | 32 | A | A | A | A | A | A | AA | B | A | A |
| Example 1-33 | 33 | A | A | A | A | A | A | AA | B | A | A |
| Example 1-34 | 34 | A | A | A | A | A | A | AA | A | A | A |
| Example 1-35 | 35 | A | A | A | A | A | A | AA | B | A | A |
| Example 1-36 | 36 | A | A | A | A | A | A | AA | A | A | A |
| Example 1-37 | 37 | A | A | A | A | A | B | A | B | A | A |
| Example 1-38 | 38 | A | A | | A | A | B | A | B | A | A |
| Comparative | C-1 | B | A | A | B | B | B | D | D | E | E |
| Example 1 | | | | | | | | | | | |
| Comparative | C-2 | B | B | B | B | B | B | E | E | C | E |
| Example 2 | | | | | | | | | | | |
| Comparative | C-3 | B | B | B | A | A | B | D | E | D | C |
| Example 3 | | | | | | | | | | | |
| Comparative | C-4 | B | A | A | B | A | B | E | D | D | E |
| Example 4 | | | | | | | | | | | |
| Comparative | C-5 | B | B | A | B | A | B | E | E | E | E |
| Example 5 | | | | | | | | | | | |
| Comparative | C-6 | B | A | A | B | A | B | D | D | D | E |
| Example 6 | | | | | | | | | | | |

As is clear from the results in the table, it was confirmed that the photoelectric conversion element according to the embodiment of the present invention has excellent quantum efficiency.

On the other hand, the photoelectric conversion element of the comparative example using the comparative compound not corresponding to the specific compound had an insufficient quantum efficiency.

In addition, from the results in Table 1, it was confirmed that in a case where the specific compound has the substituent S (in a case in Formula (1), X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—), the quantum efficiency and responsiveness are more excellent (comparison of Examples 1-15 to 1-20, and the like).

It was confirmed that in a case where the substituent selected from the substituent group S in the specific compound represents a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{Ar1}, a group represented by Formula (S-3), or a group represented by Formula (S-4), the quantum efficiency is more excellent (comparison between Example 1-1 and Example 1-27, and the like).

In the specific compound, it was confirmed that in a case where the group represented by Formula (A-1) is a group represented by Formula (A-2), the quantum efficiency is excellent (comparison between Examples 1-25 and 1-26, and the like).

It was confirmed that in a case where the group represented by Formula (A-2) is a group represented by Formula (C-1) or a group represented by Formula (C-2), the quantum efficiency is more excellent (comparison between Examples 1-10 to 1-14 and Example 1-26, and the like). In addition, it was confirmed that in a case where the group represented by Formula (A-2) is a group represented by Formula (C-1), the response speed (responsiveness) is more excellent (comparison between Example 1-3 and Example 1-4, and the like).

In Formula (1), it was confirmed that in a case where X represents >NR^{N}, >CR^{C1}R^{C2}, or >C═CR^{C3}R^{C4}, the quantum efficiency is more excellent (comparison between Examples 1-10 to 1-14 and Examples 1-15 to 1-17, and the like). In addition, it was confirmed that in a case where X represents >NR^{N}, the quantum efficiency is further excellent (comparison between Examples 1-1 to 1-9 and Examples 1-10 or 1-12, and the like).

From the comparison between Example 1 and Example 2, it was confirmed that in a case where A¹ and A² are groups represented by Formula (C-1), the response speed is more excellent.

### [Test Y]

### <Production of Photoelectric Conversion Element>

A photoelectric conversion film was formed by co-vapor depositing each of the specific compound or each of the comparative compound, the n-type organic semiconductor (fullerene (C₆₀)), the p-type organic semiconductor (Compound (P-1)), and the coloring agent, which were shown in Table 2, in a ratio of compound:coloring agent:p-type organic semiconductor:n-type organic semiconductor=1:1:2:2 in terms of a single layer by a vacuum deposition method, and a photoelectric conversion element of each of Examples and each of Comparative Examples was produced in the same manner for the other procedures as in Test X.

### <Dark Current>

The dark current was measured in the same manner as in Test X.

As a result, it was confirmed that all of the photoelectric conversion elements had a dark current of 50 nA/cm² or less, which indicates that all of the photoelectric conversion elements had a sufficiently low dark current.

### <Quantum Efficiency>

The quantum efficiency of each of the obtained photoelectric conversion elements was measured by the following method.

A voltage was applied to each photoelectric conversion element such that the electric field strength was 2.0×10⁵ V/cm, and then light was emitted from the upper electrode (transparent conductive film) side to evaluate the quantum efficiency at a wavelength of 460 nm or a wavelength of 600 nm. The quantum efficiency was evaluated according to the following standard by using the value obtained in accordance with the following expression (S4).

In Formula (S4), the quantum efficiencies of the denominator and the numerator are the quantum efficiencies at the same wavelength. In addition, for Examples and Comparative Examples shown in Table 2, Examples 2-15 was adopted as the following standard example.

quantum efficiency (relative ratio)=(quantum efficiency of each of Examples or each of Comparative Examples at wavelength of 460 nm or wavelength of 600 nm)/(quantum efficiency of the reference example at wavelength of 460 nm or wavelength of 600 nm) Expression (S4):

### (Evaluation Standard)

The evaluation standard for the quantum efficiency at a wavelength of 460 nm is as follows.
AA: quantum efficiency of 1.6 or more
A: quantum efficiency of 1.4 or more and less than 1.6
B: quantum efficiency of 1.2 or more and less than 1.4
C: quantum efficiency of 0.9 or more and less than 1.2
D: quantum efficiency of 0.5 or more and less than 0.9
E: quantum efficiency of less than 0.5

### (Evaluation Standard)

The evaluation standard for the quantum efficiency at a wavelength of 600 nm is as follows.
A: quantum efficiency of 1.6 or more
B: quantum efficiency of 1.2 or more and less than 1.6
C: quantum efficiency of 0.8 or more and less than 1.2
D: quantum efficiency of 0.4 or more and less than 0.8
E: quantum efficiency of less than 0.4

### <Response Speed>

The response speed of each obtained photoelectric conversion element was evaluated by the following method.

A voltage was applied to the photoelectric conversion element to have a strength of 2.0×10⁵ V/cm. Thereafter, the LED was turned on for an instant to emit light from the upper electrode (transparent conductive film) side, the photocurrent at this time at a wavelength of 460 nm or a wavelength of 600 nm was measured with an oscilloscope, a rise time until the signal intensity rose from 0% to 97% signal intensity was measured. The quantum efficiency was evaluated according to the following standard by using the value obtained in accordance with the following expression (S5).

In Expression (S5), the rising time of the denominator and the rising time of the numerator are the rising time at the same wavelength. In addition, for Examples and Comparative Examples shown in Table 2, Examples 2-15 was adopted as the following standard example.

relative response speed=(rise time at a wavelength of 460 nm or wavelength of 600 nm in each of Examples or each of Comparative Examples)/(rise time at a wavelength of 460 nm or wavelength of 600 nm in reference example) Expression (S5):

### (Evaluation Standard)

A: relative response speed of less than 0.5
B: relative response speed of 0.5 or more and less than 1.0
C: relative response speed of 1.0 or more and less than 1.5
D: relative response speed of 1.5 or more and less than 2.0
E: relative response speed of 2.0 or more

### <Electric Field Strength Dependence of Response Speed>

For each of the obtained photoelectric conversion elements, the electric field strength dependence of the response speed was evaluated by the following method.

The response speed at 7.5×10⁴ V/cm was measured by the same procedure as in the evaluation of the response speed of Test Y, except that the voltage applied to each photoelectric conversion element was changed to 7.5×10⁴ V/cm. The electric field strength dependence of the response speed was evaluated according to the following standard using the value obtained according to the expression (S6).

In Expression (S6), each photoelectric conversion element in the numerator and the denominator are the same.

electric field strength dependence of response speed=(rise time at 7.5×10⁴ V/cm at a wavelength of 460 nm or a wavelength of 600 nm in each of Examples or each of Comparative Examples)/(rise time at 2.0×10⁵ V/cm at a wavelength of 460 nm or a wavelength of 600 nm in each of Examples or each of Comparative Examples) Expression (S6):

### (Evaluation Standard)

A: electric field strength dependence of the response speed of less than 2.0
B: electric field strength dependence of the response speed of 2.0 or more and less than 3.0
C: electric field strength dependence of the response speed of 3.0 or more and less than 4.0
D: electric field strength dependence of the response speed of 4.0 or more and less than 5.0
E: electric field strength dependence of the response speed of 5.0 or more

Table 2 shows the evaluation results of Test Y.

Each notation in Table 2 is as described above for each notation in Table 1.

TABLE 3

| | | | | | | | | | Evaluation | | | | | |
|---|---|---|---|---|---|---|---|---|---|---|---|---|---|---|
| | | | | | | | | | | | | | Electric | Electric |
| | | | | | | | | | | | | | field strength | field strength |
| | Specific compound or comparative compound | | | | | | | | | | | | dependence | dependence |
| | | Combined | | | Formula (A-1) = | | X = | | Quantum | Quantum | Response | Response | of the response | of the response |
| | | coloring | Substituent S | Formula (A-1) = | Formula (C-1) or | X = having | NR^{N} or | X = | efficiency | efficiency | speed | speed | speed | speed |
| Table 2 | Compound | agent | definition 1 | Formula (A-2) | Formula (C-2) | substituent S | CR₂ | NR^{N} | (460 mm) | (600 nm) | (450 nm) | (600 nm) | (460 nm) | (600 nm) |
| Example 2-1 | 4 | B-1 | A | A | A | A | A | A | AA | A | A | A | A | A |
| Example 2-2 | 4 | B-2 | A | A | A | A | A | A | AA | A | A | A | A | A |
| Example 2-3 | 4 | B-3 | A | A | A | A | A | A | AA | A | A | A | A | A |
| Example 2-4 | 4 | B-4 | A | A | A | A | A | A | AA | A | A | A | A | A |
| Example 2-5 | 4 | B-5 | A | A | A | A | A | A | AA | A | A | A | A | A |
| Example 2-6 | 4 | B-6 | A | A | A | A | A | A | AA | A | A | A | A | A |
| Example 2-7 | 4 | B-7 | A | A | A | A | A | A | AA | A | A | A | A | A |
| Example 2-8 | 4 | B-8 | A | A | A | A | A | A | AA | A | A | A | A | A |
| Example 2-9 | 1 | B-8 | A | A | A | A | A | A | AA | A | B | A | A | A |
| Example 2-10 | 6 | B-8 | A | A | A | A | A | A | AA | A | A | A | A | A |
| Example 2-11 | 27 | B-8 | B | A | A | A | A | B | B | B | B | B | B | B |
| Example 2-12 | 14 | B-8 | A | A | A | A | A | B | A | A | B | A | A | A |
| Example 2-13 | 15 | B-8 | A | A | A | A | B | B | B | B | B | B | B | B |
| Example 2-14 | 17 | B-8 | A | A | A | A | B | B | B | B | B | B | B | B |
| Example 2-15 | 18 | B-8 | — | A | A | B | B | B | C | C | C | C | B | B |
| Example 2-16 | 21 | B-8 | A | A | A | A | A | B | B | A | B | A | A | A |
| Example 2-17 | 23 | B-8 | A | A | A | A | A | B | B | A | B | A | A | A |
| Example 2-18 | 25 | B-8 | A | B | B | A | A | B | C | B | C | B | B | B |
| Comparative | C-1 | B-8 | B | A | A | B | B | B | D | C | D | C | E | C |
| Example 2-1 | | | | | | | | | | | | | | |
| Comparative | C-2 | B-8 | B | B | B | B | B | B | E | D | E | A | C | E |
| Example 2-2 | | | | | | | | | | | | | | |
| Comparative | C-3 | B-8 | B | B | B | A | A | B | D | E | E | E | D | D |
| Example 2-3 | | | | | | | | | | | | | | |
| Comparative | C-4 | B-8 | B | A | A | B | A | B | E | D | D | D | D | D |
| Example 2-4 | | | | | | | | | | | | | | |
| Comparative | C-5 | B-8 | B | B | A | B | A | B | E | E | E | E | E | E |
| Example 2-5 | | | | | | | | | | | | | | |
| Comparative | C-6 | B-8 | B | A | A | B | A | B | D | D | D | D | D | D |
| Example 2-6 | | | | | | | | | | | | | | |

From the results shown in the table, it was confirmed that the desired effects were obtained in the photoelectric conversion element according to the embodiment of the present invention.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 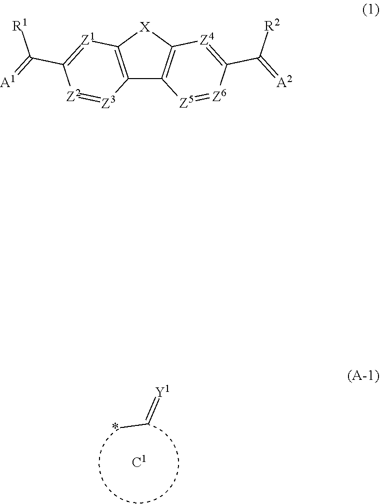
in Formula (1),
X represents >NR^{N}, >CR^{C1}R^{C2}, >C═CR^{C3}R^{C4}, >SiR^{C5}R^{C6}, >GeR^{C7}R^{C8}, —OC(R^{C9})(R^{C10})—, a sulfur atom, an oxygen atom, or a selenium atom,
R^{N} represents a substituent selected from a substituent group S,
R^{C1} to R^{C10} each independently represent a hydrogen atom or a substituent selected from the substituent group S, provided that at least one of R^{C1} or R^{C2} represents the substituent selected from the substituent group S, at least one of R^{C3} or R^{C4} represents the substituent selected from the substituent group S, at least one of R^{C5} or R^{C6} represents the substituent selected from the substituent group S, at least one of R^{C7} or R^{C8} represents the substituent selected from the substituent group S, and at least one of R^{C9} or R^{C10} represents the substituent selected from the substituent group S, R^{C1} and R^{C2}, R^{C3} and R^{C4}, R^{C5} and R^{C6}, R^{C7} and R^{C8}, and R^{C9} and R^{C10} may each independently be bonded directly or through a linking group to form a ring,
Z¹ to Z⁶ each independently represent —CR^{X1}═ or a nitrogen atom, in a case where adjacent two of Z¹ to Z⁶ are —CR^{X1}═, two R^{X1}'s may be bonded to each other to form a ring,
R^{X1} represents a hydrogen atom or a substituent,
R¹ and R² each independently represent a hydrogen atom or a substituent, and
A¹ and A² each independently represent a group represented by Formula (A-1),
in Formula (1-A),
* represents a bonding position,
C¹ represents a ring which contains 2 or more carbon atoms and may have a substituent,
Y¹ represents a sulfur atom, an oxygen atom, ═NR^{X2}, or ═CR^{X3}R^{X4},
R^{X2} represents a hydrogen atom or a substituent,
R^{X3} and R^{X4} each independently represent a cyano group, —SO₂R^{X5}, —COOR^{X6}, or —COR^{X7}, and
R^{X5} to R^{X7} each independently represent an aliphatic hydrocarbon group which may have a substituent, an aromatic ring group which may have a substituent, or an aliphatic heterocyclic group which may have a substituent,
the substituent group S: a linear aliphatic hydrocarbon group having 1 to 3 carbon atoms, a cyclic aliphatic hydrocarbon group having 3 to 8 carbon atoms, which may have a substituent, a linear aliphatic hydrocarbon group having 1 to 3 carbon atoms, which has a cyclic aliphatic hydrocarbon group having 3 to 8 carbon atoms, a linear aliphatic hydrocarbon group having 1 to 3 carbon atoms, which has an aromatic ring group which may have a substituent, a branched aliphatic hydrocarbon group having 3 carbon atoms, which has a cyclic aliphatic hydrocarbon group having 3 to 8 carbon atoms, a branched aliphatic hydrocarbon group having 3 carbon atoms, which has an aromatic ring group which may have a substituent, an aromatic ring group which may have a substituent, a group represented by Formula (S-1), and a group represented by Formula (S-2),
in the substituent group S, the linear aliphatic hydrocarbon group having 1 to 3 carbon atoms, the cyclic aliphatic hydrocarbon group having 3 to 8 carbon atoms, which may have a substituent, the linear aliphatic hydrocarbon group having 1 to 3 carbon atoms, which has a cyclic aliphatic hydrocarbon group having 3 to 8 carbon atoms, the linear aliphatic hydrocarbon group having 1 to 3 carbon atoms, which has an aromatic ring group which may have a substituent, the branched aliphatic hydrocarbon group having 3 carbon atoms, which has a cyclic aliphatic hydrocarbon group having 3 to 8 carbon atoms, and the branched aliphatic hydrocarbon group having 3 carbon atoms, which has an aromatic ring group which may have a substituent, may have an ethereal oxygen atom and may be substituted with a halogen atom,
*-L^{S1}-C(R^{S1})₃ Formula (S-1)
*—C(=Q)R^{Ac1} Formula (S-2)
in Formula (S-1),
* represents a bonding position,
L^{S1} represents a single bond or a linear alkylene group having 1 to 3 carbon atoms,
R^{S1}'s each independently represent a hydrogen atom, a linear aliphatic hydrocarbon group having 1 to 3 carbon atoms, a branched aliphatic hydrocarbon group having 3 or 4 carbon atoms, or a cyclic alkyl group having 3 carbon atoms,
a plurality of R^{S1}'s may be the same as or different from each other, provided that two or more of three R^{S1}'s are not hydrogen atoms, and
the alkylene group, the linear aliphatic hydrocarbon group having 1 to 3 carbon atoms, the branched aliphatic hydrocarbon group having 3 or 4 carbon atoms, and the cyclic alkyl group having 3 carbon atoms may have an ethereal oxygen atom and may be substituted with a halogen atom,
in Formula (S-2),
* represents a bonding position,
Q represents an oxygen atom or a sulfur atom, and
R^{Ac1} represents an aliphatic hydrocarbon group which may have a substituent, an aromatic ring group which may have a substituent, or an aliphatic heterocyclic group which may have a substituent.

2. The photoelectric conversion element according to claim 1,
wherein the substituent selected from the substituent group S represents a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{A}M, a group represented by Formula (S-3), or a group represented by Formula (S-4),
the substituent group R^{Ar1}: a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, an aromatic ring group, a halogen atom, and *—Si(R^{Si})₃, where * represents a bonding position,
R^{Si} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, or an aromatic ring group, a plurality of R^{Si}'s may be the same as or different from each other, and
in the substituent group R^{Ar1}, the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have an ethereal oxygen atom and may be substituted with a halogen atom,
*—C(R^{S2})₃ Formula (S-3)
*—C(═O)R^{Ac2} Formula (S-4)
in Formula (S-3),
* represents a bonding position,
R^{S2}'s each independently represent a hydrogen atom, a methyl group, an isopropyl group, or a t-butyl group, and
a plurality of R^{S2}'s may be the same as or different from each other, provided that the number of carbon atoms in the group represented by Formula (S-3) is 3 to 9, and two or more of three R^{S2}'s are not hydrogen atoms,
in Formula (S-4),
* represents a bonding position, and
R^{Ac2} represents a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 6 carbon atoms, which may have a halogen atom, or an aromatic ring group which may have a substituent selected from the substituent group R^{Ar1}.

3. The photoelectric conversion element according to claim 1,
wherein the group represented by Formula (A-1) is a group represented by Formula (A-2), 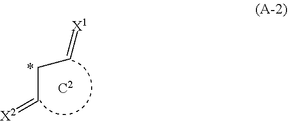
in Formula (A-2),
* represents a bonding position,
C² represents a ring containing 3 or more carbon atoms, and
X¹ and X² each independently represent an oxygen atom or a sulfur atom.

4. The photoelectric conversion element according to claim 3,
wherein the group represented by Formula (A-2) is a group represented by Formula (C-1) or a group represented by Formula (C-2), 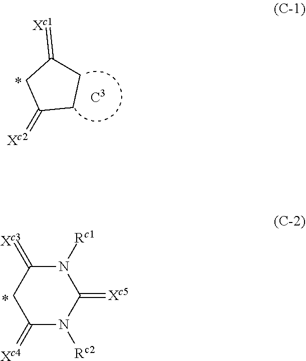
in Formulae (C-1) and (C-2),
* represents a bonding position,
in Formula (C-1),
X^{c1} and X^{c2} each independently represent a sulfur atom or an oxygen atom, and
C³ represents an aromatic ring which may have a substituent,
in Formula (C-2),
X^{c3} to X^{c5} each independently represent a sulfur atom or an oxygen atom, and
R^{c1} and R^{c2} each independently represent a hydrogen atom or a substituent.

5. The photoelectric conversion element according to claim 1,
wherein X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—, and
R^{N} and R^{C1} to R^{C10} have the same meanings as R^{N} and R^{C1} to R^{C10} in Formula (1).

6. The photoelectric conversion element according to claim 1,
wherein X represents >NR^{N}, >CR^{C1}R^{C2}, or >C═CR^{C3}R^{C4}, and
R^{N} and R^{C1} to R^{C4} have the same meanings as R^{N} and R^{C1} to R^{C4} in Formula (1).

7. The photoelectric conversion element according to claim 1,
wherein X represents >NR^{N}, and
R^{N} has the same meaning as R^{N} in Formula (1).

8. The photoelectric conversion element according to claim 1,
wherein the photoelectric conversion film further contains an n-type organic semiconductor, and
the photoelectric conversion film has a bulk hetero structure formed in a state where the compound represented by Formula (1) and the n-type organic semiconductor are mixed.

9. The photoelectric conversion element according to claim 8,
wherein the n-type organic semiconductor includes fullerenes selected from the group consisting of a fullerene and a derivative of the fullerene.

10. The photoelectric conversion element according to claim 1,
wherein the photoelectric conversion film further contains a coloring agent.

11. The photoelectric conversion element according to claim 1,
wherein the photoelectric conversion film further contains a p-type organic semiconductor.

12. The photoelectric conversion element according to claim 1, further comprising:
one or more interlayers between the conductive film and the transparent conductive film, in addition to the photoelectric conversion film.

13. An imaging element comprising:
the photoelectric conversion element according to claim 1.

14. An optical sensor comprising:
the photoelectric conversion element according to claim 1.

15. A compound represented by Formula (1), 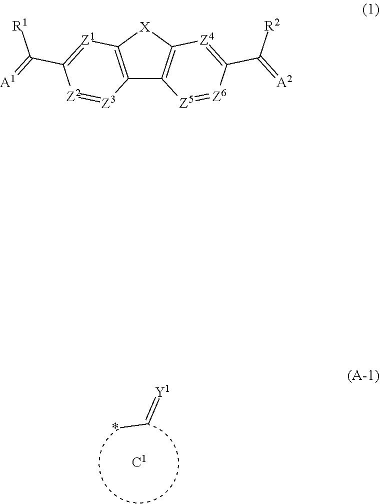
in Formula (1),
X represents >NR^{N}, >CR^{C1}R^{C2}, >C═CR^{C3}R^{C4}, >SiR^{C5}R^{C6}, >GeR^{C7}R^{C8}, —OC(R^{C9})(R^{C10})—, a sulfur atom, an oxygen atom, or a selenium atom,
R^{N} represents a substituent selected from a substituent group S,
R^{C1} to R^{C10} each independently represent a hydrogen atom or a substituent selected from the substituent group S, provided that at least one of R^{C1} or R^{C2} represents the substituent selected from the substituent group S, at least one of R^{C3} or R^{C4} represents the substituent selected from the substituent group S, at least one of R^{C5} or R^{C6} represents the substituent selected from the substituent group S, at least one of R^{C7} or R^{C8} represents the substituent selected from the substituent group S, and at least one of R^{C9} or R^{C10} represents the substituent selected from the substituent group S, R^{C1} and R^{C2}, R^{C3} and R^{C4}, R^{C5} and R^{C6}, R^{C7} and R^{C8}, and R^{C9} and R^{C10} may each independently be bonded directly or through a linking group to form a ring,
Z¹ to Z⁶ each independently represent —CR^{X1}═ or a nitrogen atom, in a case where adjacent two of Z¹ to Z⁶ are —CR^{X1}═, two R^{X1}'s may be bonded to each other to form a ring,
R^{X1} represents a hydrogen atom or a substituent,
R¹ and R² each independently represent a hydrogen atom or a substituent, and
A¹ and A² each independently represent a group represented by Formula (A-1),
in Formula (A-1),
* represents a bonding position,
C¹ represents a ring which contains 2 or more carbon atoms and may have a substituent,
Y¹ represents a sulfur atom, an oxygen atom, ═NR^{X2}, or ═CR^{X3}R^{X4},
R^{X2} represents a hydrogen atom or a substituent,
R^{X3} and R^{X4} each independently represent a cyano group, —SO₂R^{X5}, —COOR^{X6}, or —COR^{X7}, and
R^{X5} to R^{X7} each independently represent an aliphatic hydrocarbon group which may have a substituent, an aromatic ring group which may have a substituent, or an aliphatic heterocyclic group which may have a substituent,
the substituent group S: a linear aliphatic hydrocarbon group having 1 to 3 carbon atoms, a cyclic aliphatic hydrocarbon group having 3 to 8 carbon atoms, which may have a substituent, a linear aliphatic hydrocarbon group having 1 to 3 carbon atoms, which has a cyclic aliphatic hydrocarbon group having 3 to 8 carbon atoms, a linear aliphatic hydrocarbon group having 1 to 3 carbon atoms, which has an aromatic ring group which may have a substituent, a branched aliphatic hydrocarbon group having 3 carbon atoms, which has a cyclic aliphatic hydrocarbon group having 3 to 8 carbon atoms, a branched aliphatic hydrocarbon group having 3 carbon atoms, which has an aromatic ring group which may have a substituent, an aromatic ring group which may have a substituent, a group represented by Formula (S-1), and a group represented by Formula (S-2),
in the substituent group S, the linear aliphatic hydrocarbon group having 1 to 3 carbon atoms, the cyclic aliphatic hydrocarbon group having 3 to 8 carbon atoms, which may have a substituent, the linear aliphatic hydrocarbon group having 1 to 3 carbon atoms, which has a cyclic aliphatic hydrocarbon group having 3 to 8 carbon atoms, the linear aliphatic hydrocarbon group having 1 to 3 carbon atoms, which has an aromatic ring group which may have a substituent, the branched aliphatic hydrocarbon group having 3 carbon atoms, which has a cyclic aliphatic hydrocarbon group having 3 to 8 carbon atoms, and the branched aliphatic hydrocarbon group having 3 carbon atoms, which has an aromatic ring group which may have a substituent, may have an ethereal oxygen atom and may be substituted with a halogen atom,
*-L^{S1}-C(R^{S1})₃ Formula (S-1)
*—C(=Q)R^{Ac1} Formula (S-2)
in Formula (S-1),
* represents a bonding position,
L^{S1} represents a single bond or a linear alkylene group having 1 to 3 carbon atoms,
R^{S1}'s each independently represent a hydrogen atom, a linear aliphatic hydrocarbon group having 1 to 3 carbon atoms, a branched aliphatic hydrocarbon group having 3 or 4 carbon atoms, or a cyclic alkyl group having 3 carbon atoms,
a plurality of R^{S1}'s may be the same as or different from each other, provided that two or more of three R^{S1}'s are not hydrogen atoms, and
the alkylene group, the linear aliphatic hydrocarbon group having 1 to 3 carbon atoms, the branched aliphatic hydrocarbon group having 3 or 4 carbon atoms, and the cyclic alkyl group having 3 carbon atoms may have an ethereal oxygen atom and may be substituted with a halogen atom,
in Formula (S-2),
* represents a bonding position,
Q represents an oxygen atom or a sulfur atom, and
R^{Ac1} represents an aliphatic hydrocarbon group which may have a substituent, an aromatic ring group which may have a substituent, or an aliphatic heterocyclic group which may have a substituent.

16. The compound according to claim 15,
wherein the substituent selected from the substituent group S represents a linear aliphatic hydrocarbon group having 1 or 2 carbon atoms, a cyclic aliphatic hydrocarbon group having 3 to 6 carbon atoms, an aliphatic hydrocarbon group having 1 carbon atom, which has a cyclic aliphatic hydrocarbon group having 3 to 6 carbon atoms, an aromatic ring group which may have a substituent selected from a substituent group R^{Ar1}, a group represented by Formula (S-3), or a group represented by Formula (S-4),
the substituent group R^{Ar1}: a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, an aromatic ring group, a halogen atom, and *—Si(R^{Si})₃, where * represents a bonding position,
R^{Si} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, or an aromatic ring group, a plurality of R^{Si}'s may be the same as or different from each other, and
in the substituent group R^{A}M, the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have an ethereal oxygen atom and may be substituted with a halogen atom,
*—C(R^{S2})₃ Formula (S-3)
*—C(═O)R^{Ac2} Formula (S-4)
in Formula (S-3),
* represents a bonding position,
R^{S2}'s each independently represent a hydrogen atom, a methyl group, an isopropyl group, or a t-butyl group, and
a plurality of R^{S2}'s may be the same as or different from each other, provided that the number of carbon atoms in the group represented by Formula (S-3) is 3 to 9, and two or more of three R^{S2}'s are not hydrogen atoms,
in Formula (S-4),
* represents a bonding position, and
R^{Ac2} represents a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 6 carbon atoms, which may have a halogen atom, or an aromatic ring group which may have a substituent selected from the substituent group R^{Ar1}.

17. The compound according to claim 15,
wherein the group represented by Formula (A-1) is a group represented by Formula (A-2), 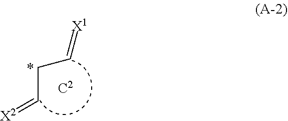
in Formula (A-2),
* represents a bonding position,
C² represents a ring containing 3 or more carbon atoms, and
X¹ and X² each independently represent an oxygen atom or a sulfur atom.

18. The compound according to claim 17,
wherein the group represented by Formula (A-2) is a group represented by Formula (C-1) or a group represented by Formula (C-2), 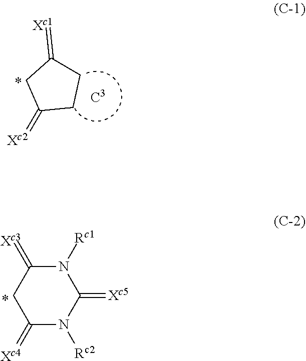
in Formulae (C-1) and (C-2),
* represents a bonding position,
in Formula (C-1),
X^{c1} and X^{c2} each independently represent a sulfur atom or an oxygen atom, and
C³ represents an aromatic ring which may have a substituent,
in Formula (C-2),
X^{c3} to X^{c5} each independently represent a sulfur atom or an oxygen atom, and
R^{c1} and R^{c2} each independently represent a hydrogen atom or a substituent.

19. The compound according to claim 15,
wherein X represents >NR^{N}, >CR^{C1}R^{C2}, >C═CR^{C3}R^{C4}, >SiR^{C5}R^{C6}, >GeR^{C7}R^{C8}, or —OC(R^{C9})(R^{C10})—, and
R^{N} and R^{C1} to R^{C10} have the same meanings as R^{N} and R^{C1} to R^{C10} in Formula (1).

20. The compound according to claim 15,
wherein X represents >NR^{N}, >CR^{C1}R^{C2}, or >C═CR^{C3}R^{C4}, and
R^{N} and R^{C1} to R^{C4} have the same meanings as R^{N} and R^{C1} to R^{C4} in Formula (1).

21. The compound according to claim 15,
wherein X represents >NR^{N}, and
R^{N} has the same meaning as R^{N} in Formula (1).

22. A compound represented by Formula (2), 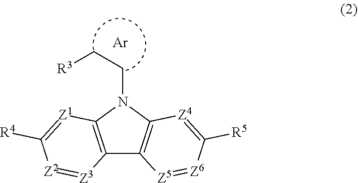
in Formula (2),
Z¹ to Z⁶ each independently represent —CR^{X1}═ or a nitrogen atom,
R^{X1} represents a hydrogen atom or a substituent,
in a case where adjacent two of Z¹ to Z⁶ are —CR^{X1}═, two R^{X1}'s may be bonded to each other to form a ring,
R³ represents a substituent selected from a substituent group T,
the substituent group T: a linear aliphatic hydrocarbon group, a branched aliphatic hydrocarbon group, a cyclic aliphatic hydrocarbon group, and an aromatic ring group which may have a substituent selected from a substituent group R^{Ar3} and does not contain a nitrogen atom,
in the substituent group T, the linear aliphatic hydrocarbon group, the branched aliphatic hydrocarbon group, and the cyclic aliphatic hydrocarbon group may have an ethereal oxygen atom,
the substituent group R^{Ar3}: a linear aliphatic hydrocarbon group, a branched aliphatic hydrocarbon group, a cyclic aliphatic hydrocarbon group, a halogen atom, and aromatic ring group which does not contain a nitrogen atom,
in the substituent group R^{Ar3}, the linear aliphatic hydrocarbon group, the branched aliphatic hydrocarbon group, and the cyclic aliphatic hydrocarbon group may have an ethereal oxygen atom and may be substituted with a halogen atom,
R⁴ and R⁵ each independently represent an iodine atom, *—O—S(═O)₂R^{f}, a bromine atom, a chlorine atom, a fluorine atom, a formyl group, *—Sn(R^{Sn})₃, *—B(R^{B1})₂, or *—B⁻(R^{B2})₃M⁺,
R^{f} represents a perfluoroalkyl group having 1 to 6 carbon atoms, R^{Sn}, R^{B1}, and R^{B2} each independently represent a substituent, and a plurality of R^{Sn}'s, R^{B1}'s, and R^{B2}'s may be the same as or different from each other, R^{B1}'s and R^{B2}'s may be bonded to each other to form a ring structure, M⁺ represents a monovalent metal cation, * represents a bonding position,
Ar represents an aromatic ring which includes two or more carbon atoms as a ring member atom and does not include a nitrogen atom as a ring member atom, the aromatic ring represented by Ar may be substituted with a substituent selected from the substituent group T or with a halogen atom,
in a case where the aromatic ring represented by Ar has the substituent selected from the substituent group T, the substituent selected from the substituent group T, which is represented by R³, and the substituent selected from the substituent group T, which is contained in the aromatic ring represented by Ar, may be bonded to each other to form a non-aromatic ring, and
in a case where the aromatic ring represented by Ar is substituted with a plurality of substituents selected from the substituent group T, the plurality of substituents may be bonded to each other to form a non-aromatic ring.

23. A manufacturing method of a compound, comprising:
a step of reacting a compound represented by Formula (2a) with a compound represented by Formula (X) to manufacture a compound represented by Formula (2b), 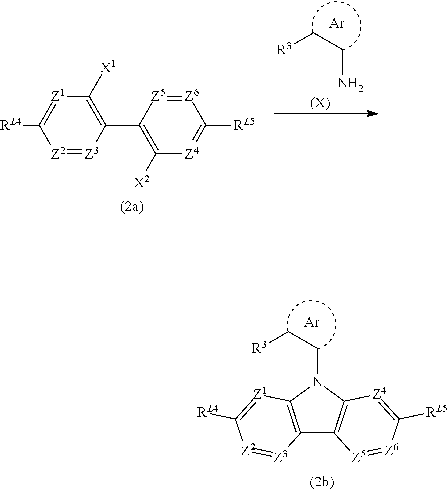
in Formula (2a),
Z¹ to Z⁶ each independently represent —CR^{X1}═ or a nitrogen atom,
R^{X1} represents a hydrogen atom or a substituent,
in a case where adjacent two of Z¹ to Z⁶ are —CR^{X1}═, two R^{X1}'s may be bonded to each other to form a ring,
R^{L4} and R^{L5} each independently represent *—O—S(═O)₂R^{f}, a bromine atom, a chlorine atom, or a fluorine atom,
X¹ and X² each independently represent an iodine atom, *—O—S(═O)₂R^{f}, a bromine atom, or a chlorine atom, and R^{f} represents a perfluoroalkyl group having 1 to 6 carbon atoms,
provided that R^{L4}, R^{L5}, X¹, and X² satisfy the following requirement,
the requirement: in a case where an iodine atom is assigned to a first rank, *—O—S(═O)₂R^{f} is assigned to a second rank, a bromine atom is assigned to a third rank, a chlorine atom is assigned to a fourth rank, and a fluorine atom is assigned to a fifth rank, and in a case where the ranks are higher from the first rank to the fifth rank, both a rank of a group represented by R^{L4} and a rank of a group represented by R^{L5} are higher than a rank of a group represented by X¹ and are higher than a rank of a group represented by X²,
in Formula (X),
R³ represents a substituent selected from the substituent group T,
the substituent group T: a linear aliphatic hydrocarbon group, a branched aliphatic hydrocarbon group, a cyclic aliphatic hydrocarbon group, and an aromatic ring group which may have a substituent selected from a substituent group R^{Ar3} and does not contain a nitrogen atom,
in the substituent group T, the linear aliphatic hydrocarbon group, the branched aliphatic hydrocarbon group, and the cyclic aliphatic hydrocarbon group may have an ethereal oxygen atom,
the substituent group R^{Ar3}: a linear aliphatic hydrocarbon group, a branched aliphatic hydrocarbon group, a cyclic aliphatic hydrocarbon group, a halogen atom, and aromatic ring group which does not contain a nitrogen atom,
in the substituent group R^{Ar3}, the linear aliphatic hydrocarbon group, the branched aliphatic hydrocarbon group, and the cyclic aliphatic hydrocarbon group may have an ethereal oxygen atom and may be substituted with a halogen atom,
Ar represents an aromatic ring which contains two or more carbon atoms as a ring member atom and does not contain a nitrogen atom as a ring member atom, the aromatic ring represented by Ar may be substituted with a substituent selected from the substituent group T or with a halogen atom,
in a case where the aromatic ring represented by Ar has the substituent selected from the substituent group T, the substituent selected from the substituent group T, which is represented by R³, and the substituent selected from the substituent group T, which is contained in the aromatic ring represented by Ar, may be bonded to each other to form a non-aromatic ring, and
in a case where the aromatic ring represented by Ar is substituted with a plurality of substituents selected from the substituent group T, the plurality of substituents may be bonded to each other to form a non-aromatic ring,
in Formula (2b),
Z¹ to Z⁶, R^{L4}, and R^{L5} have the same meanings as Z¹ to Z⁶, R^{L4}, and R^{L5} in Formula (2a), and
R³ and Ar have the same meanings as R³ and Ar in Formula (X).

24. A manufacturing method of a compound, comprising:
a step of reacting a compound represented by Formula (2a) with a compound represented by Formula (X) to manufacture a compound represented by Formula (2b); and
a step of converting a group represented by R^{L4} and a group represented by R^{L5} in the compound represented by Formula (2b) into a formyl group, *—Sn(R^{Sn})₃, *—B(R^{B1})₂, or *—B⁻(R^{B2})₃M⁺,
R^{Sn}, R^{B1}, and R^{B2} each independently represent a substituent, and a plurality of R^{Sn}'s, R^{B1}'s, and R^{B2}'s may be the same as or different from each other, R^{B1}'s and R^{B2}'s may be bonded to each other to form a ring structure, M⁺ represents a monovalent metal cation, and * represents a bonding position, 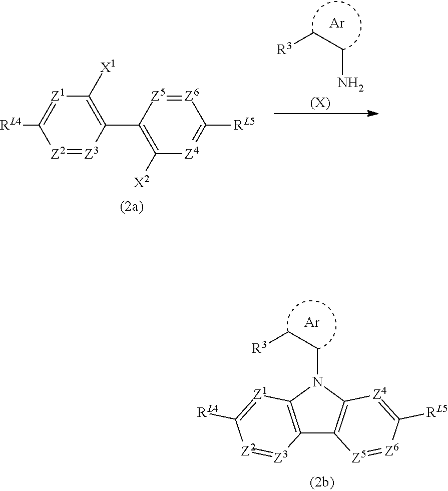
in Formula (2a),
Z¹ to Z⁶ each independently represent —CR^{X1}═ or a nitrogen atom,
R^{X1} represents a hydrogen atom or a substituent,
in a case where adjacent two of Z¹ to Z⁶ are —CR^{X1}═, two R^{X1}'s may be bonded to each other to form a ring,
R^{L4} and R^{L5} each independently represent *—O—S(═O)₂R^{f}, a bromine atom, a chlorine atom, or a fluorine atom,
X¹ and X² each independently represent an iodine atom, *—O—S(═O)₂R^{f}, a bromine atom, or a chlorine atom, and R^{f} represents a perfluoroalkyl group having 1 to 6 carbon atoms,
provided that R^{L4}, R^{L5}, X¹, and X² satisfy the following requirement,
the requirement: in a case where an iodine atom is assigned to a first rank, *—O—S(═O)₂R^{f} is assigned to a second rank, a bromine atom is assigned to a third rank, a chlorine atom is assigned to a fourth rank, and a fluorine atom is assigned to a fifth rank, and in a case where the ranks are higher from the first rank to the fifth rank, both a rank of a group represented by R^{L4} and a rank of a group represented by R^{L5} are higher than a rank of a group represented by X¹ and are higher than a rank of a group represented by X²,
in Formula (X),
R³ represents a substituent selected from the substituent group T,
the substituent group T: a linear aliphatic hydrocarbon group, a branched aliphatic hydrocarbon group, a cyclic aliphatic hydrocarbon group, and an aromatic ring group which may have a substituent selected from a substituent group R^{M} and does not contain a nitrogen atom,
in the substituent group T, the linear aliphatic hydrocarbon group, the branched aliphatic hydrocarbon group, and the cyclic aliphatic hydrocarbon group may have an ethereal oxygen atom,
the substituent group R^{Ar3}: a linear aliphatic hydrocarbon group, a branched aliphatic hydrocarbon group, a cyclic aliphatic hydrocarbon group, a halogen atom, and aromatic ring group which does not contain a nitrogen atom,
in the substituent group R^{Ar3}, the linear aliphatic hydrocarbon group, the branched aliphatic hydrocarbon group, and the cyclic aliphatic hydrocarbon group may have an ethereal oxygen atom and may be substituted with a halogen atom,
Ar represents an aromatic ring which contains two or more carbon atoms as a ring member atom and does not contain a nitrogen atom as a ring member atom, the aromatic ring represented by Ar may be substituted with a substituent selected from the substituent group T or with a halogen atom,
in a case where the aromatic ring represented by Ar has the substituent selected from the substituent group T, the substituent selected from the substituent group T, which is represented by R³, and the substituent selected from the substituent group T, which is contained in the aromatic ring represented by Ar, may be bonded to each other to form a non-aromatic ring, and
in a case where the aromatic ring represented by Ar is substituted with a plurality of substituents selected from the substituent group T, the plurality of substituents may be bonded to each other to form a non-aromatic ring,
in Formula (2b),
Z¹ to Z⁶, R^{L4}, and R^{L5} have the same meanings as Z¹ to Z⁶, R^{L4}, and R^{L5} in Formula (2a), and
R³ and Ar have the same meanings as R³ and Ar in Formula (X).

25. A compound represented by Formula (3), 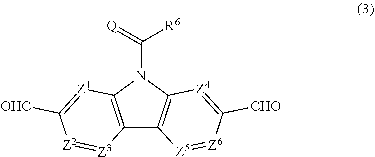
in Formula (3),
Z¹ to Z⁶ each independently represent —CR^{X1}═ or a nitrogen atom,
R^{X1} represents a hydrogen atom or a substituent,
in a case where adjacent two of Z¹ to Z⁶ are —CR^{X1}═, two R^{X1}'s may be bonded to each other to form a ring,
Q represents an oxygen atom or a sulfur atom, and
R⁶ represents a substituent selected from a substituent group U,
the substituent group U: an aliphatic hydrocarbon group which may have a substituent, an aromatic ring group which may have a substituent, and an aliphatic heterocyclic group which may have a substituent.

26. A manufacturing method of a compound, comprising:
a step 1 of reacting a compound represented by Formula (3a) with a compound represented by Formula (A) to obtain a compound represented by Formula (3b), which has a protective group represented by SiR^{Y1}₃;
a step 2 of reacting the compound represented by Formula (3b) with a metalating reagent, reacting the reacted compound with a formylating agent, and further deprotecting the protective group to obtain a compound represented by Formula (3c); and
a step 3 of reacting the compound represented by Formula (3c) with a compound represented by Formula (C) to obtain a compound represented by Formula (3), 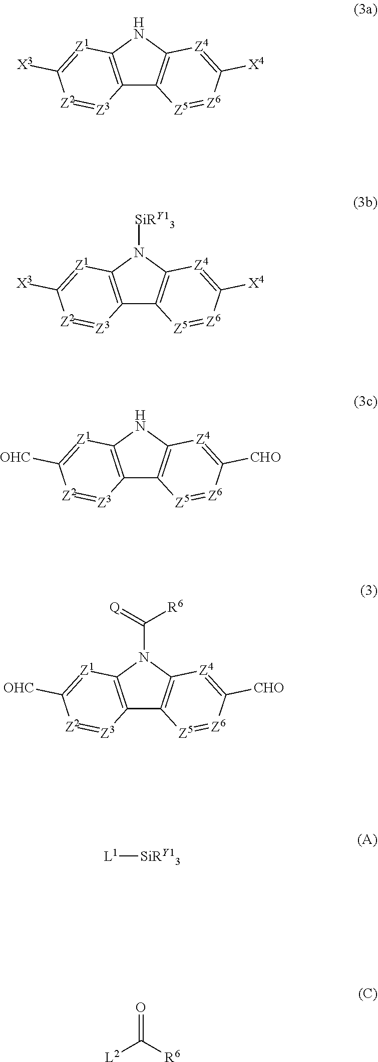
in Formulae (3a) to (3c),
Z¹ to Z⁶ each independently represent —CR^{X1}═ or a nitrogen atom,
R^{X1} represents a hydrogen atom or a substituent,
in a case where adjacent two of Z¹ to Z⁶ are —CR^{X1}═, two R^{X1}'s may be bonded to each other to form a ring,
X³ and X⁴ each independently represent an iodine atom, *—O—S(═O)₂R^{f}, a bromine atom, or a chlorine atom, and R^{f} represents a perfluoroalkyl group having 1 to 6 carbon atoms,
in Formula (3),
Z¹ to Z⁶ have the same meanings as Z¹ to Z⁶ in Formulae (3a) to (3c),
Q represents an oxygen atom or a sulfur atom, and
R⁶ represents a substituent selected from a substituent group U,
the substituent group U: an aliphatic hydrocarbon group which may have a substituent, an aromatic ring group which may have a substituent, and an aliphatic heterocyclic group which may have a substituent,
in Formula (A),
L¹ represents a leaving group,
in Formula (3) and Formula (A),
R^{Y1} represents an aliphatic hydrocarbon group which may have a substituent, an aromatic ring group which may have a substituent, or an aliphatic heterocyclic group which may have a substituent,
in Formula (C),
Q and R⁶ have the same meanings as Q and R⁶ in Formula (3), and
L² represents a leaving group.

27. A compound represented by Formula (3c), 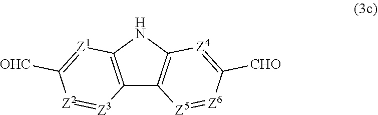
in Formula (3c),
Z¹ to Z⁶ each independently represent —CR^{X1}═ or a nitrogen atom,
R^{X1} represents a hydrogen atom or a substituent, and
in a case where adjacent two of Z¹ to Z⁶ are —CR^{X1}═, two R^{X1}'s may be bonded to each other to form a ring.
